(19) United States
(12) Patent Application Publication
(10) Pub. No.: US 2012/0053080 A1
(43) Pub. Date: Mar. 1, 2012

(54) PROTEIN MARKERS IDENTIFICATION FOR GASTRIC CANCER DIAGNOSIS

(76) Inventors: Juan Cui, Athens, GA (US); J. David Puett, Athens, GA (US); Seulgi Hong, Duluth, GA (US); Ying Xu, Bogart, GA (US)

(21) Appl. No.: 13/255,527

(22) PCT Filed: Feb. 19, 2010
(86) PCT No.: PCT/US10/24830

§ 371 Date: Nov. 9, 2011

Related U.S. Application Data
(60) Provisional application No. 61/158,683, filed on Mar. 9, 2009.
(60) Provisional application No. 61/241,347, filed on Sep. 10, 2009.

(51) Int. Cl. C40B 30/04; C12Q 1/37; C07K 14/78; C12N 9/64; C12N 9/90; C12N 9/20; G01N 33/574; C07K 14/47

(52) U.S. Cl. 506/9; 436/501; 435/23; 530/395; 435/226; 435/233; 530/350; 435/198; 530/356; 435/7.4

(57) ABSTRACT

Methods for detecting cancer as well as methods of diagnosis of cancer by detecting proteins secreted into biological fluids are disclosed The invention was first applied to detecting proteins secreted into serum and urine However, it is understood that the methods have broader application to developing tools and systems for detecting proteins secreted into other biological fluids such as, but not limited to, saliva, spinal fluid, seminal fluid, vaginal fluid, and ocular fluid Reliable detection of proteins secreted into biological fluids provided by embodiments of the methods will enable more timely and accurate detection and diagnosis of cancer.

### BACKGROUND OF THE INVENTION

### Field of the Invention

The present invention is generally directed to methods of detecting protein markers in biological fluids of a patient for the detection and/or diagnosis of cancer.

### BACKGROUND

One of the main challenges in the field of cancer is to be able detect cancers in the early stages. Challenges in early cancer detection came mainly from the reality that most cancers do not have clear physical symptoms at their early stage that may implicate the cancer. Physical exams like mammography or colonoscopy proved to be effective but have been limited to only certain types of cancers such as breast or colorectal cancer. Moreover, the cancer may already be beyond the early stage when detected through such physical exams, even when these are conducted on a regular basis. It is all too frequent that a cancer is diagnosed when it is already in an advanced stage; clearly, more effective techniques for early cancer detection are needed.

Alterations in gene and protein expression provide important clues about the physiological states of a tissue or an organ. During malignant transformation, genetic alterations in tumor cells can disrupt autocrine and paracrine signaling networks, leading to the over-expression of some classes of proteins such as growth factors, cytokines and hormones that may be secreted outside of the cancerous cells (Hanahan and Weinberg, 2000; Sporn and Roberts, 1985). These and other secreted proteins may get into serum, saliva, blood, urine, cerebrospinal (spinal) fluid, seminal fluid, vaginal fluid, ocular fluid, or other biological fluids through complex secretion pathways.

While the tissue marker genes can be useful for grading a cancer if the cancer has been detected, they are not directly useful for cancer diagnosis, unless a specific cancer is being suspected and the relevant tissue is being probed. Protein markers from biological fluids are really the ultimate goal for marker identification because they allow cancer detection through simple analytical tests.

However, identification of cancer markers (proteins, peptides or other molecules) in biological fluids (for example, serum) represents a much more challenging problem compared to gene expression studies of cancer tissues, because of the greater complexity of the molecular composition and the wide dynamic range of the abundance of the molecules in human serum, possibly as high as 6 orders of magnitude in difference ranging from mg/ml to ng/ml. The human serum proteome, for example, is a very complex mixture of highly abundant native serum proteins such as albumin and immunoglobulins, as well as proteins and peptides that are secreted from different tissues, diseased or normal, or leak from cells throughout the human body (Adkins et al., 2002; Schrader et al., 2001). Many factors such as disease, diet and even mental status can change the molecular composition and their abundance in the serum rather quickly. Compounding these issues, most of the circulating native blood proteins are orders of magnitude more abundant than those of most of the secreted proteins. These issues have made it exceedingly difficult to carry out direct comparative analyses of proteomes from biological fluids of patients and reference population for biomarker identification.

Recent advances in genomic and proteomic techniques have generated much enthusiasm and new hope for identifying effective markers for early detection of cancer. Through comparative analyses of gene expression patterns in cancer versus reference tissues using techniques like microarray chips, one can possibly detect consistent changes in the expression patterns of some genes in cancer versus normal tissues, even for cancer at its very early stage. This is possible because as cancer develops through the key developmental stages, it will acquire a number of new capabilities such as (a) self-sufficiency in growth signals, (b) insensitivity to antigrowth signals, (c) evasion of apoptosis, (d) limitless replication potential, (e) sustained angiogenesis and (f) tissue invasion and metastasis, each of which will alter the “normal” expression patterns of some genes, e.g., increase their expression levels to produce the relevant proteins needed for the acquired capabilities; and some of these proteins can be secreted into the blood circulation, providing possible traces useful for cancer detection through blood tests.

Using the omics techniques, a number of markers in both cancer tissue and serum have been proposed. Mass spectrometry has been the main technique for proteomic studies of proteins in biological fluids such as serum, particularly for identification and quantification of proteins in biological fluids such as serum (Tolson et al., 2004).

Global patterns of expressed proteins could be useful for some cases but they are clearly not good markers because of the high complexity of the global patterns of expressed proteins.

The general consensus in the field is that the current markers are not working effectively, and fundamentally new ideas are needed to identify more effective markers for cancer detection, particularly at its early stage.

An additional problem that exists in the field is that in order to diagnose cancers and other diseases, accurate predictions must be made regarding which proteins from abnormally expressed genes in diseased tissues (such as cancers) can be secreted into biological fluids. A difficulty associated with solving this problem is that current understanding of downstream localization after proteins are secreted outside of cells is very limited and the current knowledge is not sufficient to provide useful hints about secretion of proteins to biological fluids. Accordingly, what is needed is a data classification method for predicting which proteins would likely be secreted into biological fluids.

We believe that integrating the information derivable from microarray data of cancer tissues with proteomic studies conducted on biological fluids using computational methods represents a novel and more effective approach to finding new and more effective markers in a more systematic manner.

### SUMMARY

Methods for detecting cancer as well as methods of diagnosis of cancer by detecting proteins secreted into biological fluids are disclosed. Reliable detection of proteins secreted into biological fluids provided by embodiments of the present invention will enable more timely and accurate detection and diagnosis of cancer.

In one embodiment, the invention discloses a method for determining protein markers for the detection of cancer, the method comprising: a) obtaining a cancer sample and a reference sample; b) determining one or more genes that are differentially expressed between the cancer sample and the reference sample; c) identifying one or more proteins that are the products of said one or more genes; d) predicting the probability of the one or more proteins being secreted into a biological fluid; and e) detecting in the biological fluid, the presence of the one or more proteins that are predicted to be secreted into the biological fluid, wherein the detection of the one or more proteins in the biological fluid constitutes detection of cancer.

In another embodiment, the invention discloses a method of diagnosing a patient with cancer, comprising: a) obtaining a biological fluid from the patient; and b) detecting in the biological fluid, the presence of one or more marker proteins, wherein the one or more marker proteins are the products of one or more genes that are differentially expressed between a cancer sample and a reference sample, wherein the one or more marker proteins are predicted and experimentally validated to be secreted into the biological fluid, and wherein the detection of the one or more marker proteins in the biological fluid constitutes detection of cancer.

In a third embodiment, the invention discloses a method of diagnosing a subject with cancer, the method comprising: a) obtaining a biological fluid from the subject; and b) measuring a level of one or more marker proteins in the biological fluid, wherein the one or more marker proteins are the products of one or more genes that are differentially expressed between a cancer sample and a reference sample, wherein the one or more marker proteins are predicted and experimentally validated to be secreted into the biological fluid, and wherein the differential expression of the one or more marker proteins in the biological fluid relative to the standard level is indicative of cancer.

In yet another embodiment, the invention discloses markers for cancer identification comprising one or more proteins selected from the group consisting of MUC13, GKN2, COL10A, AZTP1, CTSB, LIPF, GIF, EL, and TOP2A, wherein the differential expression of the one or more proteins in a biological fluid obtained from a subject relative to a standard level is indicative of the occurrence of cancer in the subject.

In another embodiment, the invention discloses kits for detecting cancer in a subject comprising: (a) one or more first antibodies that specifically bind to proteins in the biological fluid, wherein the proteins are selected from the group consisting of MUC13, GKN2, COL10A, AZTP1, CTSB, LIPF, GIF, EL, and TOP2A; (b) a second antibody that specifically binds to the one or more of the first antibodies; and optionally, (c) a reference sample.

To illustrate the present invention, the invention was first applied to detecting proteins secreted into serum and urine. However, it is understood that the present invention has broader application to developing tools and systems for detecting proteins secreted into other biological fluids such as, but not limited to, saliva, spinal fluid, seminal fluid, vaginal fluid, and ocular fluid.

### BRIEF DESCRIPTION OF THE DRAWINGS/FIGURES

FIG. 1 shows (a) a schematic for selection of the probe selection regions (PSRs) across the entire length of a transcript. The short dashes underneath the PSR represent individual probes for each PSR (Source: Affymetrix: GeneChip® Exon Array System for Human, Mouse, and Rat). Lighter regions denote exons and the darker regions represent introns that are removed during splicing. (b) PCR data for three predicted splicing isoforms. The x-axis is the tissue sample axis (12 tissue samples), where NC is for negative control. The Y-axis is the mass axis. (i) One isoform with exon 2 skipped; and (ii) two isoforms with an alternative exon 2 (lower) and with exon 1 (upper) skipped, respectively. (c) A schematic of exon isoforms and probes. The long horizontal line represents a portion of the human genome, the narrowest rectangle represents an exon, and three broader rectangles represent three exon isoforms, and the shorter black lines in the bottom represent probes.

FIG. 2 illustrates (a) Venn diagram of the total 2,540 genes differentially expressed in cancer versus reference tissues, and 1,276 genes differentially expressed in early stage cancers. (b) Distribution of expression differentials across the 2,540 genes between cancer and reference tissues.

FIG. 3 illustrates (a) Functional family distributions of the 2,540 differentially expressed genes, 911 cancer-related genes and 1,276 genes differentially expressed in early stage cancer. (b) Subcellular location distributions of the above three groups of genes (*Cyt.: Cytoplasm; Nuc.: Nucleus; E.R.: Endoplasmic Reticulum; Pla.: Plasma Membrane; Ext.: Extracellular Space).

FIG. 4 illustrates (top) the expression level of MUC1 in cancer tissues changes as a function of age, which is independent of gender; (bottom) expression of THY1 is independent of both age and gender.

FIG. 5 illustrates identified bi-clusters across 80 samples over subsets of genes, where each row represents a gene and each column represent a pair of cancer/reference tissues. (a) C1 (top) has 244 genes that are consistently up-regulated in cancer versus reference tissues; C2 (middle) has 95 genes, most of which are down-regulated; C3 (bottom) has 53 genes, showing complex patterns. Note that the order of the tissue samples for different bi-clusters is not necessarily the same since the algorithm rearranges the order of tissue samples. (b) A bi-cluster possibly subtype-specific, consisting of 42 genes. The six genes marked with the vertical bar are known to be associated with a subtype of gastric cancer.

FIG. 6 illustrates a Box diagram showing distribution of the matched motifs in the immediate upstream intronic region (−150 nt, +30 nt) with the occurrence of the predicted exon-skipping events.

FIG. 7(a) The curve marked with vertical lines represents the overall accuracies of k-gene markers (k=1, . . . , 100), which is the average of the best accuracies of 500 randomly selected subsets; the curve marked with crosses represents the best 5-cross validation accuracy of k-gene markers (k=1, . . . , 8), identified through an exhaustive search. (b) The heat-map for the best 28-gene marker, which comprises of 13 up-regulated and 15 down-regulated genes. Among them, NKAP, TMEM185B, C14orf104, and C1orf96 are up-regulated, while KLF15, PI16, and GADD45B are down-regulated across >89% early stage patients.

FIG. 8 illustrates MS total ion chromatograms of pooled serum samples from the control and cancer groups (a) Base peaks of the control group on the left and base peaks of the cancer group on the right; (b) For different molecular weight ranges.

FIG. 9 illustrates Western blots (SDS-PAGE followed by transfer to nitrocellulose for subsequent blotting with antibody) for eight proteins: MUC13, GKN2, COL10A1, AZTP1, CTSB, LIPF, GIF, and TOP2A, showing differences in abundance between the control group and gastric cancer group. 1) MUC13 (1 μg, dilution: 1st Ab 1:200; 2nd Ab Anti-rabbit, 1:10,000); 2) GKN2 (150 μg, dilution: 1st Ab 1:1,000; 2nd Ab Anti-rabbit, 1:30,000); 3) COL10A1(1 μg, dilution: 1st Ab 1:500; 2nd Ab Anti-rabbit, 1:10,000); 4) AZTP1 (120 μg, dilution: 1st Ab 1:500; 2nd Ab Anti-mouse, 1:3,000); 5) CTSB (5 μg, dilution: 1st Ab 1:1,500; 2nd Ab Anti-rabbit, 1:20,000); 6) LIPF (120 μg, dilution: 1st Ab 1:500; 2nd Ab Anti-goat, 1:10,000); 7) GIF (120 μg, dilution: 1st Ab 1:5,00; 2nd Ab Anti-mouse, 1:3,000); and 8) TOP2A (60 μg, dilution: 1st Ab 1:350; 2nd Ab Anti-goat, 1:10,000).

FIG. 10 illustrates the statistical relationship between the d and the p-value=P(TP), d represents to the distance from the separating hyperplane between the positive and the negative training data.

FIG. 11 illustrates enriched functional groups as by the Database for Annotation, Visualization and Integrated Discovery (DAVID). DAVID provides a comprehensive set of functional annotation tools to understand the biological meaning behind large lists of genes. The x-axis represents the functional groups, and the y-axis represents the enrichment.

FIG. 12 illustrates the enriched pathways for 480 predicted urine proteins using the KEGG Orthology-based Annotation System (KOBAS) web server. KOBAS identifies the frequently occurring (or significantly enriched) pathways among queried sequences compared against a background distribution. The shorter bar in each group represents the percentage of the 480 proteins; the longer bar in each group indicates all human proteins; the x-axis indicates the pathway names; and the y-axis.

FIG. 13 illustrates the underrepresented pathways for the 480 proteins. The shorter bar in each group indicates the percentage of the 480 proteins; the longer bar in each group indicates all human proteins; the x-axis indicates the pathway names; and the y-axis indicates the percentage.

FIG. 14 illustrates 274 cytokine antibody array for 3 normal samples (N1, N2, N3) and 3 gastric cancer samples (SCE SC5, SC11). Human G6 Array shows Fit3-ligand (white rectangle); Human G7 Array shows EGF-R (dark grey rectangle), SOP-130 (white rectangle); Human G8 Array shows PDGF-AA (white rectangle); Human G9 Array shows Trappin-2 (light grey rectangle), Lutenizing Hormone (white rectangle), TIM-1 (dark grey rectangle); Human G10 Array shows CEACAM1 (light grey rectangle), FSH (white rectangle), CEA (dark grey rectangle).

FIG. 15 illustrates Western blot for Mucin13 for three cancer samples (GC) and three control samples (CTRL). Each lane contains 1 μg of urinary protein. Santa Cruz Mucin 13 (M−250) rabbit polycolonal antibody was used in 1:200 dilution; the anti-rabbit secondary antibody was used in 1:10,000 dilution.

FIG. 16 illustrates Western blot for COLA10A 1 for three control samples (CTRL) and three cancer samples (GC). Each lane contains 1 μg of urinary protein. The Calbiochem Anti-Collagen Type X Rabbit pAb was used in 1:200 dilution; Anti-rabbit secondary antibody was used in 1:10,000 dilution.

FIG. 17 (upper) Western blot for Endothelial Lipase (EL) on three control samples (CTRL) and three stomach cancer samples (GC). Each lane is 1 μg of urinary proteins. Antibody used for EL was Santa Cruz EL (C-19) affinity purified goat polycolonal antibody (1:200 dilution); Anti-goat secondary antibody was used in 1:15,000 dilution. (lower) The first 7 lanes correspond to normal samples; last 7 lanes are cancer samples.

FIG. 18 depicts classification performance by the best one-gene and two-gene markers for prostate cancer and the control data. The y-axis is the classification accuracy and the x-axis is the list of top 100 markers sorted by their classification accuracies.

FIG. 19 shows the results of protein array experiments using the Biotin label-based antibody arrays. FIG. 19 illustrates the distribution of protein abundance differentials across the 103 proteins between cancer and reference sera, with the x-axis representing the list of the 103 proteins sorted in the increasing order of the log-values of their abundance differentials and the y-axis being the log-values of the abundance differentials.

The present invention will now be described with reference to the accompanying drawings. It is understood that the drawings of the present application are not necessarily drawn to scale and that these figures and illustrations merely illustrate, but do not limit, the present invention.

### DETAILED DESCRIPTION OF THE INVENTION

The present invention is directed to methods for detecting cancer by predicting whether proteins are secreted into a biological fluid such as, but not limited to, serum, saliva, blood, urine, spinal fluid, seminal fluid, vaginal fluid, and ocular fluid, and validating the prediction by determining the presence of such proteins in the biological fluid in proteomic studies, wherein the detection of such proteins in the biological fluid constitutes detection of cancer. The present invention includes method embodiments for diagnosing a patient with cancer by detecting, in a biological fluid of the patient, the presence of one or more marker proteins expressed from abnormally expressed genes in cancer tissues, wherein the marker proteins are predicted and experimentally validated to be secreted into the biological fluid, and wherein the detection of the marker proteins in the biological fluid constitutes detection of cancer.

Any of a variety of biological fluids are amenable to analysis using the devices and methods of the present invention. Such fluids include cerebrospinal fluid, synovial fluid, blood, serum, plasma, saliva, intestinal fluids, semen, tears, nasal secretions, etc. It will be appreciated that any fluidic biological sample (e.g., tissue or biopsy extracts, extracts of feces, sputum, etc.) may likewise be employed in accordance with the present invention.

In the following description, for purposes of explanation, specific numbers, parameters and reagents are set forth in order to provide a thorough understanding of the invention. It is understood, however, that the invention may be practiced without these specific details. In some instances, well-known features may be omitted or simplified so as not to obscure the present invention.

The embodiment(s) described, and references in the specification to “one embodiment”, “an embodiment of the invention”, “an embodiment”, “an example embodiment”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is known in the art to effect such feature, structure, or characteristic in connection with other embodiments whether or not explicitly described.

The description of “a” or “an” item herein may refer to a single item or multiple items. For example, the description of a feature, a protein, a biological fluid, or a classifier may refer to a single feature, a protein, a biological fluid, or a classifier. Alternatively, the description of a feature, a protein, a biological fluid, or a classifier may refer to multiple features, proteins, biological fluids, or classifiers. Thus, as used herein, “a” or “an” may be singular or plural. Similarly, references to and descriptions of plural items may refer to single items.

It is understood that wherever embodiments are described herein with the language “comprising,” otherwise analogous embodiments described in terms of “consisting of and/or “consisting essentially of are also provided.

The specification describes general approaches for detecting and diagnosing cancer by detecting the presence of marker proteins in a biological fluid. Specific exemplary embodiments for detecting marker proteins in the serum are provided herein. This specification discloses one or more embodiments that incorporate the features of this invention. The disclosed embodiment(s) merely exemplify the invention. The scope of the invention is not limited to the disclosed embodiment(s). The invention is defined by the claims appended hereto.

Although the claimed methods and their corresponding description in the specification generally claim the feature of detecting a protein marker for the detection of a cancer, it is understood that analyzing a sample for the presence of such protein markers and finding no such marker proteins and, thus, no diagnosis of cancer is still detecting the presence of the protein markers.

### DEFINITIONS

The terms “polypeptide,” “peptide,” “protein”, and “protein fragment” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, a “protein” or “peptide” generally refers, but is not limited to, a protein of greater than about 200 amino acids up to a full length sequence translated from a gene; a polypeptide of about 100 to 200 amino acids; and/or a “peptide” of from about 3 to about 100 amino acids. As used herein, an “amino acid” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

As used herein, a “cancer” in a subject or patient refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

As used herein, a “sample” refers to a sample of biological material obtained from a patient, preferably a human patient, including a tissue, a tissue sample, a cell sample, e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), a tumor sample or RNA extracted from the tissue sample. Samples can also be biological fluid samples, including but not limited to, urine, blood, serum, platelets, saliva, cerebrospinal fluid, nipple aspirates, and cell lysate (e.g. supernatant of whole cell lysate, microsomal fraction, membrane fraction, or cytoplasmic fraction). The sample may be obtained using any methodology known in the art.

By “biological sample” is intended any biological sample obtained from an individual, including but not limited to, a fecal (stool) sample, biological fluid (e.g., blood), cell, tissue sample, RNA sample, or tissue culture. Methods for obtaining stool samples, tissue biopsies and other biological samples from mammals are well known in the art.

As used herein, a “tissue sample” refers to a portion, piece, part, segment, or fraction of a tissue which is obtained or removed from an intact tissue of a subject.

The term “gene” refers to a nucleic acid (e.g., DNA) sequence that comprises coding sequences necessary for the production of a polypeptide, precursor, or RNA (e.g., rRNA, tRNA). The term “gene” encompasses both cDNA and genomic forms of a gene.

A genomic form or clone of a gene contains the coding region or “exons” interrupted with non-coding sequences termed “introns” or “intervening regions” or “intervening sequences.” Introns are removed or “spliced out” from the nuclear or primary transcript; introns therefore are absent in the messenger RNA (mRNA) transcript. In addition to containing introns, genomic forms of a gene can also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript).

It is understood that “intron” and “exon” are relative with respect to a particular mRNA spliced variant, and that an exon of one spliced variant may be an intron of another, and vice versa. However, within one spliced variant, an “intron” cannot be an “exon” and vice versa. These terms “intron” and “exon” are used herein for convenience and clarity and are not meant to be limiting.

As used herein, the term “gene expression” refers to the process of converting genetic information encoded in an endogenous gene, ORF or portion thereof, or a transgene in plants into RNA (e.g., mRNA, rRNA, tRNA, or snRNA) through “transcription” of the endogenous gene, ORF or portion thereof, or a transgene in plants (e.g., via the enzymatic action of an RNA polymerase), and for protein encoding genes, into protein through “translation” of mRNA. In addition, expression refers to the transcription and stable accumulation of sense (mRNA) or functional RNA. Gene expression can be regulated at many stages in the process. “Up-regulation” or “activation” refers to regulation that increases the production of gene expression products (e.g., RNA or protein), while “down-regulation” or “repression” refers to regulation that decrease production. Molecules (e.g., transcription factors) that are involved in up-regulation or down-regulation are often called “activators” and “repressors,” respectively.

The terms “differentially expressed gene,” “differential gene expression,” and their synonyms, which are used interchangeably, refer to a gene whose expression is activated to a higher or lower level in a subject suffering from a disease, specifically cancer, such as gastric cancer, relative to its expression in a normal or control subject. The terms also include genes whose expression is activated to a higher or lower level at different stages of the same disease. It is also understood that a gene that is differentially expressed may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about 1.5-fold, two-fold, preferably at least about four-fold, more preferably at least about six-fold, most preferably at least about ten-fold difference between the expression of a given gene in normal and diseased subjects, or in various stages of disease development in a diseased subject.

As used herein, the term “subject” or “patient” refers to any animal (e.g., a mammal), including, but not limited to humans, non-human primates, rodents, and the like, suspected of having cancer or which is to be the subject of a particular diagnosis. Typically, the terms “subject” and “patient” are used interchangeably herein in reference to a human subject.

As used herein, a “normal subject” or “control subject” refers to a subject not suffering from a disease.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition of or an absence of tumor metastasis; inhibition or an absence of tumor growth; relief of one or more symptoms associated with the specific cancer; reduced morbidity and mortality; improvement in quality of life; or some combination of effects.

As used herein, the term “classifier” refers to a method, algorithm, computer program, or system for performing data classification.

As used herein, the term “classification” is the process of learning to separate data points into different classes by finding common features between collected data points which are within known classes. Classification can be done using neural networks, regression analysis, or other techniques.

As used herein, the term “data classification methods” represent a general class of computational methods that attempt to determine which pre-defined classes each data element in a given data set belongs to, based on the provided feature values of each data element.

The term “antibody-based binding moiety” or “antibody” includes immunoglobulin molecules and immunologically active determinants of immunoglobulin molecules, e.g., molecules that contain an antigen binding site which specifically binds (immunoreacts with) protein. The term “antibody-based binding moiety” is intended to include whole antibodies, e.g., of any isotype (IgG, IgA, IgM, IgE, etc), and includes fragments thereof which are also specifically reactive with prohibitn, or fragments thereof. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)2, Fab′, Fv, dAbs and single chain antibodies (scFv) containing a VL and VH domain joined by a peptide linker. The scFv's may be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or FITS.

In one aspect of the present invention a method is provided for determining serum protein markers for the detection of cancer, the method comprising: a) obtaining a cancer sample and a reference sample; b) determining one or more genes that are differentially expressed between the cancer sample and the reference sample; c) identifying one or more proteins that are the products of said one or more genes; d) predicting the probability of the one or more proteins being secreted into a biological fluid; and e) detecting in the biological fluid, the presence of the one or more proteins that are predicted to be secreted into the biological fluid, wherein the detection of the one or more proteins in the biological fluid constitutes detection of cancer.

Cancer samples and reference samples can be obtained from the same subject or from different subjects. The “reference sample” refers to a sample containing a baseline amount of the expression of one or more genes as determined in one or more normal subjects that does not have cancer. A baseline may be obtained from at least one subject and is preferably obtained from an average of subjects (e.g., n=2 to 100 or more), wherein the subject or subjects have no prior history of cancer. A baseline can also be obtained from one or more normal samples from a subject suspected to have cancer. For example, a baseline may be obtained from at least one normal sample and is preferably obtained from an average of normal samples (e.g., n=2 to 100 or more), wherein the subject is suspected of having cancer. In one aspect, the expression of one or more genes may be increased in the cancer sample as compared to the reference sample. In another aspect, the expression of one or more genes may be decreased in the cancer sample as compared to the reference sample.

### Analysis of Gene Expression

Determining one or more genes that are differentially expressed between the cancer sample and the reference sample involves isolating nucleic acid from the cancer sample and the reference sample. The nucleic acid sample may be total RNA, a cDNA sample, poly(A) RNA, an RNA sample depleted of one or more RNAs, for example, an RNA sample depleted of rRNA or an amplification product of RNA. In one aspect the sample, is from a mammal, for example, a human, a rat, or a mouse. The sample may be isolated from a tissue, including, for example, blood, lung, heart, kidney, pancreas, prostate, testis, uterus, brain, or skin.

Genes that are differentially expressed between the cancer sample and the reference sample can be assayed by any means known in the art including, but not limited to, microarray profiling, polymerase chain reaction (PCR), methods based on hybridization analysis of polynucleotides, methods based on sequencing of polynucleotides, methods based on analysis of alternative gene splicing, and proteomics-based methods.

Widely used methods known in the art for studying gene expression by the quantification of RNA in a biological sample include microarray analysis, Northern blot analysis (Harada, 1990), and in situ hybridization (Parker & Barnes, 1999); RNAse protection assays (Hod, 1992); S1 nuclease mapping (Fujita et al., 1987) and PCR-based methods, such as reverse transcription polymerase chain reaction (RT-PCR) (Weis et al., 1992), quantitative RT-PCR and ligase chain reaction (LCR) (Barany, 1991), which are conventional methods in the art. Alternatively, antibodies may be employed that can recognize sequence-specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

In one embodiment, determining one or more genes that are differentially expressed between the cancer sample and the reference sample involves isolating total RNA from the cancer sample and the reference sample. General methods for total RNA extraction are well known in the art and are disclosed in standard textbooks of molecular biology, including Ausubel et al., Current Protocols of Molecular Biology, John Wiley and Sons (1997).

In a preferred embodiment, differentially expressed genes in cancer versus reference samples are studied using microarray analysis of the total RNA isolated from the cancer sample and the reference sample.

In another embodiment, differentially expressed genes in cancer versus reference samples are studied using Northern blot analysis.

In yet another embodiment, differentially expressed genes in cancer versus reference samples are studied using RNAse protection assays.

In another embodiment, differentially expressed genes in cancer versus reference samples are determined by assessing the expression of RNA by hybridizing isolated cellular RNA with a radiolableled synthetic DNA sequence homologous to the 5′ terminus of the RNA of interest.

In another embodiment, differentially expressed genes in cancer versus reference samples are studied using polymerase chain reaction (PCR).

In another embodiment, differentially expressed genes in cancer versus reference samples are studied using RT-PCR.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., 1996.

In lieu of PCR, alternative methods, such as the “Ligase Chain Reaction” (“LCR”) may be used to study gene expression (Barany, 1991).

Further PCR-based techniques include, for example, differential display (Liang and Pardee, 1992); amplified fragment length polymorphism (iAFLP) (Kawamoto et al., 1999); BeadArray™ technology (Illumina, San Diego, Calif.; Oliphant et al., Discovery of Markers for Disease (Supplement to Biotechniques), June 2002; Ferguson et al., 2000); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., 2001); and high coverage expression profiling (HiCEP) analysis (Fukumura et al., 2003).

In another embodiment of the invention, differentially expressed genes in cancer versus reference samples are studied by Serial Analysis of Gene Expression (SAGE).

In another embodiment of the invention, differentially expressed genes in cancer versus reference samples are studied by Massively Parallel Signature Sequencing (MESS). For a description of this method, see Brenner et al., (2000).

Previous studies on cancer markers have not been able to examine the whole human transcriptome, having left out the majority of the human transcriptome, splicing variants generated by alternative splicing of genes, due to the lack of effective techniques to study them until very recently. Therefore, in another embodiment of the invention, differentially expressed genes in cancer versus reference samples are studied by identifying differentially expressed splicing variants of genes in cancer versus reference samples.

Alternative splicing is a eukaryotic cellular process through which multiple mature mRNA transcripts can be produced from the same pre-mRNA through inclusion of different portions of exons and/or through retention of introns. It is estimated that at least 40-75% of human genes undergo alternative splicing under different conditions (Modrek and Lee, 2002). Alternative splicing is largely responsible for the complexity of the human transcriptome and proteome. Previous estimates suggest that the human proteome has at least ˜100,000 and possibly up to ˜150,000 different proteins, encoded by ˜20,000 genes, indicating that each human gene encodes 5-7 proteins on average. Thus, the majority of the functional proteins in human cells are splicing isoforms, highlighting the need to study splicing variants when studying gene expression and proteins, in the present case, marker proteins in biological fluids.

It is known that alternative splicing is involved in many biological processes in humans (Nakao et al., 2005), in both regular and aberrant functional processes. Deviant splicing can have serious implications to the normal function of a cell. A recent survey reviewed 29 mutations in p53's splicing sites having occurred in 12 cancer types (Holmila et al., 2003). Another recent study found that 464 splicing variants of ˜200 genes are differentially expressed in human prostate cancer (Li et al., 2006).

In one embodiment, the emerging exon-array technique by Affymetrix provides a powerful tool for studying alternative splicing.

Analysis of exon array data represents a challenging problem since the basic units for such arrays are exons rather than genes. From the exon array data, one can estimate the expression levels of individual exons, using methods such as Robust Multichip Average (RMA) (Irizary et al., 2003) and Probe Logarithmic Intensity Error (PLIER) estimation (Affymetrix, 2005), from which one can possibly infer the major splicing isoforms, based on the similarities of expression levels of the exons. The challenge is that in a given tissue, there could be more than one expressed splicing isoform for each gene with different expression levels so the observed expression level for each exon is the total expression level of all the expressed splicing isoforms containing this exon. The computational problem is to figure out which splicing isoforms are expressed and at what level, and the predicted results should be consistent with the exon expression data, which are often noisy. While there are computer programs designed to interpret the exon array data such as ANOVA (Affymetrix, 2005), the problem represents a new issue since exon arrays have only begun to be widely used since 2006. There is still a number of challenging and unsolved problems associated with exon array data interpretation. Among them is the key issue to reliably predict the major splicing isoforms and their expression levels.

Prediction of Proteins that can be Secreted from Tissue into Blood Circulation

Using gene expression data analysis techniques, numerous genes have been either identified or proposed to be relevant to specific cancers such as liver cancer (Smith et al., 2003), kidney cancer (Young et al., 2003), breast cancer (van der Vijver et al., 2002), colorectal cancer (Resnick et al., 2004) and other major cancers (Sallimen et al., 2000; Hendrix et al., 2001). In addition, a few markers for estimation of cancer stages have been proposed. However, by comparing the marker genes in tissues derived based on differential gene expression data and marker proteins in blood sera found through proteomic analyses, we observed that their links are rather weak, indicating a disconnection between the information generated using genomic and proteomic techniques on cancer tissue and blood serum, respectively.

Thus, while the tissue marker genes can be useful for grading a cancer if the cancer has been detected, they are not directly useful for cancer diagnosis, unless a specific cancer is being suspected and the relevant tissue is being probed. Markers obtained from biological fluids are really the ultimate goal for marker identification since they allow cancer detection through simple analytical tests. The key in successfully doing this is to find effective ways to best utilize the information derived from gene expression studies on cancer tissues to guide cancer marker identification in biological fluids.

Having a capability to predict which proteins in a diseased tissue can be secreted into biological fluids will provide a key link in bridging the information derivable from microarray expression data to identification of marker proteins in biological fluids.

Numerous studies have been carried out to predict the subcellular locations of proteins, including proteins that can get trafficked to the cell surface or secreted into the extracellular environment (Menne et al., 2000; Nair and Rost, 2005; Guda et al., 2006; Horton et al., 2007), based on protein sequence information like signal peptides, transmembrane domains of certain lengths, amino acid composition, and protein functions (Mott et al., 2002; Guda et al., 2006). While these programs can predict if a protein can be secreted from a cell, they are not concerned about where the proteins, after leaving the cell, will end up.

In the present invention, this issue has been addressed using a data mining approach by first collecting human proteins that are known to be secreted into biological fluids, such as, but not limited to, serum, urine, saliva, spinal fluid, seminal fluid, vaginal fluid, amniotic fluid, gingival crevicular fluid, and ocular fluid due to various pathological conditions, which were detected by proteomic studies, and then identifying common features present in these proteins in terms of their physical and chemical properties, as well as their sequence and structural features that can be used to predict them. Using this strategy, a computer program has been developed and reported for predicting proteins that can be secreted from tissues into biological fluids. See PCT Application No. PCT/US2009/053309, which is incorporated herein as reference in entirety.

The basic idea of the algorithm is as follows. An extensive literature search has led to a large collection of human proteins that are known to be secreted into the bloodstream due to various pathological conditions, as detected by previous proteomic studies. A list of features shared by these secreted proteins was delineated, including their physical and chemical properties, amino acid sequence and motif, and structural features (Table 1). Using these features, a classifier was trained to distinguish proteins that can be secreted into biological fluids from those that cannot. This algorithm was then used to predict which of the tissue gene markers may get secreted into biological fluids.

In one embodiment, the algorithm involves the steps of selecting a positive, secreted class of proteins; selecting representative proteins for a negative set; mapping protein features to construct a feature set; training a classifier to recognize characteristics of classes of proteins; determining accuracy and relevancy of mapped features; removing the least important features to produce a re-trained classifier; receiving protein sequences; vector generation and scaling; predicting classes for the received protein sequences; and returning a prediction result for the received protein sequences. A detailed description of the algorithm is provided in the copending application PCT/US2009/053309.

TABLE 1
A list of initial features for prediction of blood-secreted proteins

| Type of | | |
|---|---|---|
| properties | Features | Sources |
| General | Amino acid | Locally calculated. |
| sequence | composition, sequence | |
| features | length, di-peptides | |
| | composition | |
| | Normalized Moreau- | Calculated using the Protein Feature Server |
| | Broto autocorrelation, | (PROFEAT) developed by the National |
| | Moran autocorrelation, | University of Singapore's Bioinformatics & |
| | Geary autocorrelation, | Drug Design group (BIDD) within the |
| | Sequence order, Pseudo | Computational Science Department, Science |
| | amino acid composition | Faculty. |
| Physicochemical | Hydrophobicity, | Locally computed with three descriptors: |
| properties | normalized Van der | composition (C), transition (T), and distribution |
| | Waals volume, polarity, | (D). |
| | polarizability, charge, | |
| | secondary structure and | |
| | solvent accessibility | |
| | Solubility, unfoldability, | Determined with the sequence-based PROtein |
| | disorder regions, global | SOlubility evaluator (PROSO) (Smialowski et |
| | charge and | al., 2007) and the combined transmembrane |
| | hydrophobility | topology and signal peptide predictor (Phobius) |
| | | from the Stockholm Bioinformatics Centre. |
| Structural | Secondary structural | Determined using the Secondary Structural |
| properties | content, | Content Prediction (SSCP) tool from the |
| | shape (Radius Gyration) | European Molecular Biology Laboratory and |
| | | Radius of Gyration filters for globular protein |
| | | Evaluation from the Supercomputing Facility for |
| | | Bioinformatics & Computational Biology, |
| | | Indian Institute of Technology (IIT), Delhi. |
| Domains | Signal peptide, | Determined using the SignalP tool from the |
| and motifs | transmembrane domains | Center for Biological Sequence Analysis at the |
| | (alpha helix and beta | Technical University of Denmark and the amino |
| | barrel), Glycosylation | acid composition based TransMembrane Barrel- |
| | (both N-linked and O- | Hunt (TMB-Hunt) tool (Garrow et al, 2005). |
| | linked), Twin-arginine | Calculated using the NetOglyc, NetNgly, and |
| | signal peptides motif | Twin-arginine signal peptide (TatP) servers from |
| | (TAT) | the Center for Biological Sequence Analysis at |
| | | the Technical University of Denmark |

It is understood that protein features can differ for different biological fluids. Accordingly, the features listed in Table 1 can differ for different biological fluids. The protein features listed in Table 1 can be roughly grouped into four categories: (i) general sequence features such as amino acid composition, sequence length, and di-peptide composition (Bhasin and Raghava, 2004; Reczko and Bohr, 1994); (ii) physicochemical properties such as solubility, disordered regions, hydrophobicity, normalized Van der Waals volume, polarity, polarizability, and charges, (iii) structural properties such as secondary structural content, solvent accessibility, and radius of gyration, and (iv) domains/motifs such as signal peptides, transmembrane domains, and twin-arginine signal peptides motif (TAT).

In one embodiment, human proteins that are annotated as secretory proteins are collected from known protein databases, such as the Swiss-Prot and Secreted Protein Database (SPD) databases, and proteins that have been detected experimentally in blood by previous studies are selected. Chen et al. (2005) describes a web-based SPD.

According to an embodiment of the present invention, protein sequences corresponding to proteins collected from a biological fluid are received in the FASTA format.

In other embodiments of the invention, protein sequences corresponding to proteins collected from a biological fluid are received in other known formats, including, but not limited to a ‘raw’ text format comprising only alphabetic characters. In accordance with an embodiment of the invention, any white spaces, such as spaces, carriage returns, or TAB characters in received protein sequences in the raw text format are ignored.

Various supervised learning methods, such as a Support Vector Machine (SVM), artificial neural network (ANN), decision tree, regression models, and other algorithms have been widely implemented for data classification and regression models. Based on known data (knowledge in the form of a training data set), those supervised learning methods enable a computer to automatically learn to recognize complex patterns and develop a classifier, which can in turn be used for making intelligent decisions and predicting the class of unknown data (an independent set).

In one embodiment of the invention, the classifier is a Support Vector Machine (SVM). Traditional SVMs are based on the concept of decision hyperplanes that define decision boundaries. A decision hyperplane is one that separates between a set of objects having different class memberships. For example, collected objects may belong either to class one or class two and a classifier, such as an SVM can be used to determine (i.e., predict) the class (e.g., one or two) of any new object to be classified. Traditional SVMs are primarily classifier methods that perform classification tasks by constructing hyperplanes in a multidimensional space that separates cases of different class labels. SVMs can support both regression and classification tasks and can handle multiple continuous and categorical variables. In embodiments of the present invention, an SVM-based classifier is trained to predict the class of protein sequences as either being secreted or not secreted into a biological fluid.

In another embodiment of the invention, the classifier is a specialized, modified SVM-based classifier. The modified SVM-based classifier is used to efficiently calculate the probability of protein secretion into a biological fluid. The Gaussian radial basis function kernel provides superior performance to other, more traditional kernels used in SVM such as linear and polynomial kernels. Thus, in an embodiment, Gaussian kernel SVM is used for the training the classifier.

In one embodiment of the invention, the SVM-based classifier is further trained to predict if abnormally and highly expressed genes, detected by microarray gene expression experiments, will have their proteins secreted into the bloodstream. Studies have identified a number of such genes that show abnormally high expression levels in patients of various pathological conditions, such as cancers. Armed with this knowledge, the SVM-based classifier can be used to diagnose various cancers based upon calculating the probability that certain proteins will be excreted into a patient's bloodstream.

In one embodiment, based on the performance of each classifier initially trained, a feature selection process, named recursive feature elimination (RFE) (Tang et al., 2007), is used to remove features irrelevant or negligible to the classification goal.

According to one embodiment, based on the results on multiple data sets presented above, the overall prediction accuracy of predictions produced by the SVM-based classifier ranges from 79.5% to 98.1%, with at least 80% of known blood-secreted proteins correctly predicted for both independent evaluation test and the extra blood proteins test. From the independent negative evaluation test, the false positive rate is found to be ˜10%, a reasonable percentage of misclassified non-blood-secreted proteins, which is helpful in alleviating the doubts associated with low precision.

### Validation of Secreted Protein Markers

Once proteins that are secreted into biological fluids are predicted using the above algorithm, these protein markers are validated by assessing the presence of the protein markers in biological fluids of cancer patients using proteomic approaches.

The presence of a protein in the biological fluids can be measured by any means known in the art including, but not limited to, competition binding assays, mass spectrometry, Western blot, fluorescent activated cell sorting (FACS), enzyme-linked immunosorbent assay (ELISA), antibody arrays, high pressure liquid chromatography, optical biosensors, and surface plasmon resonance.

In one embodiment, the biological fluid sample is treated as to prevent degradation of protein. Methods for inhibiting or preventing degradation of proteins include, but are not limited to treatment of the biological fluid sample with protease, freezing the biological fluid sample, or placing the biological fluid sample on ice. Preferably, prior to analysis, the biological fluid samples are constantly kept under conditions as to prevent degradation of protein.

In one embodiment, the biological fluid is serum and the level of protein is determined by measuring the level of protein in the serum.

In one embodiment, the biological fluid is blood and the level of protein is determined by measuring the level of protein in platelets of the blood sample.

In one embodiment, the biological fluid is urine and the level of protein is determined by measuring the level of protein in urine.

In one embodiment, proteins most abundantly present in the biological fluid are removed prior to measuring the level of protein in the biological fluid. In one aspect, the proteins most abundantly present in the biological fluid comprise albumin, IgG, α1-acid glycoprotein, α2-macroglobulin, HDL (apolipoproteins A-1 and A-II), and fibrinogen.

In one embodiment, the proteins most abundantly present in the biological fluid are removed using an antibody column.

In one embodiment the non-specifically bound proteins are eluted from the antibody column following removal of the proteins most abundantly present in the biological fluid.

In one embodiment the specifically bound proteins are eluted from the antibody column for further analysis.

In one embodiment, the methods of the invention may be performed concurrently with methods of detection for other analytes, e.g., detection of mRNA or other protein markers associated with cancer (e.g. P-glycoprotein, β-tubulin, mutations in the β-tubulin gene, or overexpression of β-tubulin isotypes).

In one embodiment, protein is detected by contacting the biological fluid with an antibody-based binding moiety that specifically binds to protein, or to a fragment of that protein. Formation of the antibody-protein complex is then detected and measured to indicate protein levels. Anti-protein antibodies are available commercially (e.g. human protein affinity purified polyclonal and monoclonal Antibodies from R&D Systems, Inc. Minneapolis, Minn. 55413; AVIVA Systems Biology, San Diego, Calif. 92121; see also U.S. Pat. No. 5,463,026). Alternatively, antibodies can be raised against the full length protein, or a portion of protein. Antibodies for use in the present invention can also be produced using standard methods to produce antibodies, for example, by monoclonal antibody production.

In the methods of the invention that use antibody based binding moieties for the detection of a secreted protein, the level of the protein of interest present in the biological fluids correlates to the intensity of the signal emitted from the detectably labeled antibody.

In one preferred embodiment, the antibody-based binding moiety is detectably labeled by linking the antibody to an enzyme. Chemiluminescence is another method that can be used to detect an antibody-based binding moiety. Detection may also be accomplished using any of a variety of other immunoassays. For example, by radioactively labeling an antibody, it is possible to detect the antibody through the use of radioimmune assays. It is also possible to label an antibody with a fluorescent compound. Among the most commonly used fluorescent labeling compounds are CYE dyes, fluorescein isothiocyanate, rhodamine, phycoerytherin, phycocyanin, allophycocyanin, o-phthaldehyde and fluorescamine. An antibody can also be detectably labeled using fluorescence emitting metals such as ¹⁵²Eu, or others of the lanthanide series.

In other embodiments, the levels of protein in the biological fluids can be measured by immunoassays, such as enzyme linked immunoabsorbant assay (ELISA), radioimmunoassay (RIA), Immunoradiometric assay (IRMA), Western blotting, or immunohistochemistry. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

A widely used enzyme immunoassay is the “Enzyme-Linked Immunosorbent Assay (ELISA).” There are different forms of ELISA, such as “sandwich ELISA” and “competitive ELISA” which are well known in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, 1984.

Alternatively, protein levels in cells and/or tumors can be detected in vivo in a subject by introducing into the subject a labeled antibody to protein. For example, the antibody can be labeled with a radioactive marker whose presence and location in a subject can be detected by standard imaging techniques.

In one embodiment, immunohistochemistry (“IHC”) and immunocytochemistry (“ICC”) techniques are used.

For direct labeling techniques, a labeled antibody is used. For indirect labeling techniques, the sample is further reacted with a labeled substance.

Other techniques may be used to detect the levels of protein according to a practitioner's preference, based upon the present disclosure. One such technique is Western blotting (Towbin et al., 1979), wherein a suitably treated biological fluid is run on an SDS-PAGE gel before being transferred to a solid support, such as a nitrocellulose filter. In one embodiment, Western blotting is used to detect levels of protein in the serum or urine. Detectably labeled antibodies can then be used to detect and/or assess levels of the protein where the intensity of the signal from the detectable label corresponds to the amount of protein. Levels can be quantified, for example by densitometry.

In addition, protein levels may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al., 2000; Rowley et al., 2000; and Kuster and Mann, 1998). Further, mass spectrometric techniques have been developed that permit at least partial de novo sequencing of isolated proteins (see, e.g. Chait et al., 1993; Keough et al., 1999; reviewed in Bergman, 2000).

In certain embodiments, a gas phase ion spectrophotometer is used. In other embodiments, laser-desorption/ionization mass spectrometry is used to analyze the biological fluid. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”).

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4^{th} ed. Vol. 15 (John Wiley & Sons, New York 1995), pp. 1071-1094.

Detection of the presence of a protein marker will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

It is understood that, any of the components of a mass spectrometer, e.g., desorption source, mass analyzer, detect, etc., and varied sample preparations can be combined with other suitable components or preparations described herein, or to those known in the art. For example, in some embodiments a control sample may contain heavy atoms, e.g. ¹³C, thereby permitting the test sample to be mixed with the known control sample in the same mass spectrometry run.

In one preferred embodiment, a laser desorption time-of-flight (TOF) mass spectrometer is used.

In some embodiments the relative amounts of one or more proteins present in a first or second sample of a biological fluid is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the protein that is present in the first and second samples. A standard containing a known amount of a protein can be analyzed as the second sample to provide better quantify the amount of the protein present in the first sample. In certain embodiments, the identity of the proteins in the first and second sample can also be determined.

In one embodiment of the invention, levels of protein in biological fluids are detected by MALDI-TOF mass spectrometry.

Methods of detecting protein in biological fluids also include the use of surface plasmon resonance (SPR).

The SPR biosensing technology has also been combined with MALDI-TOF mass spectrometry for the desorption and identification of biomolecules.

In one embodiment, proteins in biological fluids are detected using Antibody Arrays. In a preferred embodiment, biotin label-based antibody arrays are used to detect the proteins.

In one embodiment, the invention discloses a method of diagnosing cancer in a subject comprising detecting one or more marker proteins in a biological fluid obtained from the subject.

In another embodiment, the invention discloses a method of diagnosing cancer in a subject comprising detecting the differential expression of one or more marker proteins in a biological fluid obtained from the subject relative to a standard level. In one aspect, the differential expression of the one or more marker proteins comprises an increase in the levels of the one or more proteins in the biological fluid relative to the standard level. In another aspect, the differential expression of the one or more marker proteins comprises a decrease in the levels of the one or more proteins in the biological fluid relative to the standard level.

In one embodiment, the invention discloses markers for cancer identification comprising one or more proteins selected from the group consisting of MUC13, GKN2, COL10A, AZTP1, CTSB, LIPF, GIF, EL, and TOP2A, wherein the differential expression of the one or more proteins in a biological fluid obtained from a subject relative to a standard level is indicative of the occurrence of cancer in the subject.

In one embodiment, single-gene markers were used for detection of early stage cancers.

In another embodiment, 2-gene markers were used for detection of early stage cancers.

In another embodiment, k-gene markers (k=1 . . . 8) were used for detection of early stage cancers.

In another embodiment, the invention discloses a kit for detecting cancer in a subject comprising: (a) a reference sample comprising a biological fluid obtained from a normal subject; (b) a solution comprising one or more first antibodies that specifically bind to proteins in the biological fluid, wherein the proteins are selected from the group consisting of MUC13, GKN2, COL10A, AZTP1, CTSB, LIPF, GIF, EL, and TOP2A; and c) a solution comprising a second antibody that specifically binds to the one or more first antibodies.

Specific preferred embodiments of the present invention will become evident from the following more detailed description of certain preferred embodiments and the claims.

### EXAMPLES

The examples which follow are illustrative of specific embodiments of the invention, and various uses thereof. They are set forth for explanatory purposes only, and are not taken as limiting the invention.

### Example 1

### Sample Collection

A total of 80 gastric cancer tissues (4 in stage I, 7 in stage II, 54 in stage III and 15 in stage IV from 27 female and 53 male patients) and the same number of adjacent gastric but non-cancerous tissues were collected from the same 80 patients (tumors confined to the mucosa or submucosa). To ensure the integrity of the mRNAs used in the array experiments, all tissues were snap-frozen and stored in liquid nitrogen within 20 minutes after resection. In addition, blood samples were also collected from each of the cancer patients before surgery. All samples were collected at three affiliated hospitals of the Jilin University College of Medicine and Jilin Provincial Cancer Hospital, Changchun, China. The histological classification and pathologic staging for each tissue was determined by experienced pathologists according to the WHO criteria and the TNM classification system of the International Union against Cancer. The cancer was classified into early (stages I and II) and advanced gastric carcinomas (stages III and IV) by tumor depth. Detailed patient information such as age, gender, histo-differentiation, pathologic stage, and history of using alcohol/smoking is listed in Table 2.

TABLE 2
(a) Patient statistics. (b) Detailed information of samples collected.
(a)

Patients

| | | |
|---|---|---|
| | | Percentage |
| Characters | No. of cases | (%) |

| | | | |
|---|---|---|---|
| Gender | Female | 27 | 33.8 |
| (n = 80) | Male | 53 | 66.2 |
| Stage | I | 4 | 5.0 |
| (n = 80) | II | 7 | 8.8 |
| | III | 54 | 67.5 |
| | IV | 15 | 18.8 |
| Age | >=55 | 53 | 68.8 |
| (n = 77) | <55 | 24 | 31.2 |
| Smoking | Yes | 18 | 28.1 |
| (n = 64) | No | 46 | 71.9 |
| Alcohol | Yes | 11 | 17.2 |
| (n = 64) | No | 5.3 | 82.8 |

(b)

| Patient | | | | | | Weight |
|---|---|---|---|---|---|---|
| ID | Age | Gender | Stage | Smoking | Alcohol | (kg) |
| 1 | 41 | F | IV | 0 | 0 | 43 |
| 2 | 62 | F | III | 0 | 0 | 70 |
| 3 | 54 | F | III | 0 | 0 | 70 |
| 4 | 62 | F | IIIA | 0 | 0 | 60 |
| 5 | 63 | M | IIIB | 1 | 1 | — |
| 6 | 56 | M | IIIB | 1 | 1 | — |
| 7 | 71 | M | IIIB | 1 | 0 | — |
| 8 | 55 | F | IIIB | 0 | 0 | 63 |
| 9 | 53 | M | IIIB | 0 | 0 | 60 |
| 10 | — | M | IV | — | — | — |
| 11 | 55 | M | IIIB | 0 | 0 | 60 |
| 12 | 51 | M | IIIB | 1 | 0 | — |
| 13 | 64 | M | IIIB | 0 | 0 | 55 |
| 14 | 53 | F | IIIB | 0 | 0 | 77 |
| 15 | 56 | M | IIIB | 1 | 0 | 55 |
| 16 | 54 | M | III | 0 | 0 | 70 |
| 17 | 53 | M | III | 0 | 0 | 62 |
| 18 | 71 | M | III | 0 | 0 | 60 |
| 19 | 57 | M | IIIA | — | — | 65 |
| 20 | 58 | M | III | 0 | 0 | 50 |
| 21 | 42 | M | IB | 0 | 0 | 52 |
| 22 | 73 | M | IB | 0 | 0 | 63 |
| 23 | 69 | F | III | 0 | 0 | 50 |
| 24 | 65 | F | IIIA | 0 | 0 | — |
| 25 | 50 | M | III | 1 | 0 | 47 |
| 26 | 47 | M | IB | 1 | 1 | 65 |
| 27 | 59 | M | III | 0 | 0 | 57 |
| 28 | 75 | M | III | 0 | 0 | 65 |
| 29 | 40 | M | III | 0 | 1 | 80 |
| 30 | 69 | M | III | 0 | 0 | 55 |
| 31 | 41 | M | II | — | — | — |
| 32 | 76 | F | II | 0 | 0 | — |
| 33 | 51 | F | III | 1 | 0 | 52 |
| 34 | 36 | M | IIIA | 1 | 0 | 60 |
| 35 | 67 | F | IV | 0 | 0 | 48 |
| 36 | 42 | M | III | 0 | 0 | 60 |
| 37 | 68 | M | III | 0 | 0 | 50 |
| 38 | 65 | M | III | 0 | 1 | 50 |
| 39 | 59 | M | III | 1 | 1 | 51 |
| 40 | 68 | M | IV | 0 | 0 | 48 |
| 41 | 74 | M | IB | 0 | 0 | 62 |
| 42 | 65 | F | IIIA | 0 | 0 | 53 |
| 43 | 50 | M | III | 0 | 0 | 62 |
| 44 | 49 | M | III | 1 | 1 | 60 |
| 45 | 58 | M | IV | 0 | 0 | 66 |
| 46 | — | F | IV | — | — | — |
| 47 | 53 | F | IIIA | 1 | 0 | 60 |
| 48 | 84 | M | IV | 1 | 1 | 70 |
| 49 | 60 | F | IIIB | 0 | 0 | 60 |
| 50 | 55 | M | III | 0 | 0 | 50 |
| 51 | 70 | M | II | 1 | 0 | 59 |
| 52 | 56 | F | III | 0 | 0 | 45 |
| 53 | 43 | F | III | 0 | 0 | 55 |
| 54 | 71 | F | III | 0 | 0 | 42 |
| 55 | 56 | F | IV | — | — | — |
| 56 | 81 | M | III | 1 | 0 | 56 |
| 57 | 65 | M | III | 0 | 0 | 70 |
| 58 | 55 | M | III | 0 | 0 | 69 |
| 59 | 56 | F | II | 0 | 0 | 74 |
| 60 | 76 | M | II | 0 | 0 | 70 |
| 61 | 78 | F | III | 0 | 0 | 39 |
| 62 | 55 | M | III | 0 | 0 | 74 |
| 63 | 65 | M | III | 0 | 1 | 70 |
| 64 | 68 | M | III | 1 | 1 | 69 |
| 65 | 63 | M | IV | 0 | 0 | — |
| 66 | — | M | IV | — | — | — |
| 67 | 57 | F | III | 0 | 0 | 61 |
| 68 | 68 | F | III | — | — | — |
| 69 | 54 | M | III | 1 | 1 | 49 |
| 70 | 51 | M | II | — | — | 70 |
| 71 | 34 | M | III | 0 | 0 | 90 |
| 72 | 75 | F | IV | — | — | 40 |
| 73 | 61 | M | III | 1 | 0 | 70 |
| 74 | 54 | M | IV | — | — | — |
| 75 | 55 | M | III | — | — | — |
| 76 | 67 | F | II | — | — | — |
| 77 | 62 | F | IV | — | — | — |
| 78 | 50 | F | III | — | — | — |
| 79 | 71 | M | IV | — | — | — |
| 80 | 58 | M | IV | — | — | — |

### Example 2

### RNA Preparation and Microarray Experiment

Total RNA was extracted from cancer tissues and reference tissues using Trizol reagent (Invitrogen) followed by purification using the RNeasy Mini kit (QIAGEN) according to the manufacturer's recommendation. Ratios of A₂₆₀/A₂₈₀>1.9 and 28S/18S rRNA of 2 were used, ensuring that the RNA samples were highly purified and not degraded. The RNA samples were analyzed using the GeneChip Human Exon 1.0 ST (Affymetrix), following the protocol detailed in the Genechip Expression Analysis Technical Manual (P/N 900223) for the array experiment. In brief, 1 μg of total RNA was used as template for synthesis of cDNA after rRNA reduction and RNA concentration. Through reverse transcription in vitro, cRNA was obtained and used as the template for cDNA synthesis in the second cycle. Then cRNA was hydrolyzed by RNaseH, and the sense strand DNA was digested by two endonucleases. Fragmented samples were labeled with DNA labeling reagent. The labeled samples were mixed with hybridization cocktail and hybridized to the microarray at 45° C., 60 rpm, and incubated for 17 hours. After hybridization, the array was washed and stained on the GeneChip® Fluidics Station 450, using the appropriate fluidics script, before being inserted into the Affymetrix autoloader carousel and scanned using the GeneChip® Scanner 3000 with GeneChip® Operating Software (GCOS).

Besides RNA quality control assessment, analysis for GeneChip QC and Data QC reports was routinely done. In accordance with requirements and suggestions of Affymetrix GeneChip Quality Control documents, the quality metrics for each hybridized array, i.e., the average background, noise (Raw Q), scaling factor, percentage of present calls, and internal control genes (hybridization and polyA controls), were assessed to ensure that each array generated high-quality gene expression data. Expression Console™ software was used to compute quality assessment metrics. Principal Components Analysis (PCA) was utilized for the assessment of data quality. Two reports were generated to summarize the assessment results for GeneChip Quality Control and Data Quality Control, respectively. No outlier arrays were detected in either the GeneChip QC or Data QC analysis.

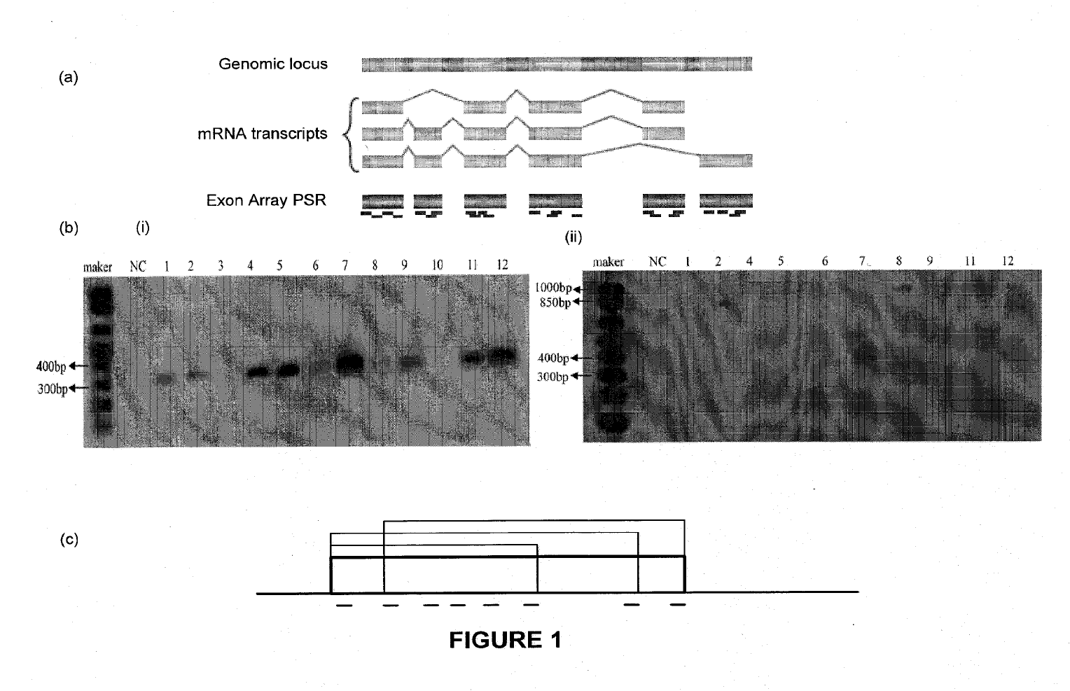

Array Design, The GeneChip Human Exon 1.0 ST array designed to be as inclusive as possible at the exon level, deriving from annotations ranging from empirical determined, highly curated mRNA sequences to ab-initio computational predictions. The array contains approximately 5.4 million 5-μm probes grouped into 1.4 million probe sets interrogating over one million exon clusters. For each exon, one or several probe selection regions (PSRs) are used, each of which is a contiguous and non-overlapping segment of the exon and has varying lengths (FIG. 1). A PSR represents a region of the genome (assembly HG18, Build 38) predicted as an integral, coherent unit of transcriptional behavior. In many cases, each PSR is an exon; in other cases, due to potentially overlapping exon structures, several PSRs may form contiguous, non-overlapping subsets of a true biological exon. A key consideration in selecting the locations of PSRs within each exon is that they can potentially reveal the alternative splicing sites used in the expressed splicing variants. For this reason, some PSRs are also used within introns of a gene in order to capture intron retentions. For each PSR, typically 4 probes are used and each is 25 base-pairs long, which are generally unique (FIG. 1). About 90% of the PSRs are represented by 4 probes (a “probe set”). Such redundancy allows robust statistical algorithms to be used in estimating presence of signal, relative expression, and existence of alternative splicing. The Affymetrix exon array includes a set of 1195 positive control probe sets representing exons of 100 housekeeping genes that are usually highly expressed in most tissues, as well as 2904 negative-control probe sets.

Hybridization takes place between each probe and the expressed mRNAs extracted from the cancer and reference tissues, each attached with a fluorescent molecule. The expression level of each PSR is estimated as the averaged intensity of the four probes placed in the region. In the present study, PLIER (Affymetrix, 2005), an algorithm that is recommended by Affymetrix, has been used for performing the estimation.

### Example 3

### Identification of Differentially Expressed Genes

The raw probe intensities for each exon was normalized using the quartile normalization approach, and the PLIER program (Affymetrix, 2005) was utilized to summarize the probe signal to both the exon- and gene-level expressions. Genes having very low expressions in either cancer or reference samples were removed; specifically, a gene was removed if its average expression level is below 10 (normalized signal intensity). To detect genes with consistent differential expression patterns in cancer versus reference tissues, a simple statistical test on the expression data was applied as follows: for each gene, Kₑₓₚ, the number of pairs of cancer/reference tissues whose expression fold change is larger than k (k is set to be 1.25 to 4, depending on specific problems) was examined; if the p-value for the observed Kₑₓₚ was less than 0.05, the gene was considered to have differential expression between the majority of the cancer and reference tissue pairs. Also, additional statistical analyses, i.e., the ANOVA test and the paired Wilcoxon signed-rank test were used to ensure that the selected genes have differential expression patterns consistently across the cancer and the reference tissue pairs.

### Example 4

### Prediction of Splice Variants Based on Exon Array Data

A novel algorithm was developed for predicting splice variants based on estimated exon expression levels. The algorithm relies on the ECgene database (Lee et al., 2007), the most comprehensive database for human transcripts, which contains 181,848 high-confidence splice variants and 129,209 medium-confidence variants, all derived from human EST data. It is assumed that all the transcripts for each gene are in ECgene so the algorithm needs to determine which ones are most probable for the given array data. ANOVA is first used to identify all differentially expressed probe selection region (PSR) patterns between the cancer and the reference tissues. Then the algorithm solves the following optimization problem.

For a given gene with n exons and m known splice variants (all in ECgene), it is required to find a subset of the m splice variants and their expression levels so that their total exon expression levels are as close as possible to the observed exon expression data. Let I be an m×n binary matrix with each row representing a spice variants and each column representing an exon, and I_{i,j}=0 if and only if variant i does not contain exon j. Let (e₁, e₂, . . . , eₙ) be the observed expression values of the n exons. It is required to find {xᵢ,} and {yᵢ, { that minimize the following (quadratic) function

$$min {\sum }_{j = 1}^{n} ( {e}_{j} - {\sum }_{i = 1}^{m} {I}_{ij} {x}_{i} {y}_{i} ) \\ Subject to \text{:} \{ \begin{matrix}{\sum }_{i = 1}^{m} {I}_{ij} {x}_{i} {y}_{i} \leq {e}_{j} , & j = 1 , … , n \\ {x}_{i} = 0 , 1 , & i = 1 , … , m ; \\ {y}_{j} > 0 , & j = 1 , … , n .\end{matrix} \qquad ( Eq . 1 )$$

where xᵢ is a binary variable and yᵢ is a real variable. This problem can be solved using the following heuristic strategy. It was first assumed that all the known splice variants are being used for the current gene, i.e., all {x₁} are set to 1. Now the problem reduces to a linear programming (LP) program (of {y,} variables in Eq. 1), which can be solved using any existing LP solver for the optimum {y₁} values, the predicted expression levels for the corresponding transcripts. To evaluate the feasibility of the assumption, the observed LP solution is tested against 100,000 solutions obtained upon all possible 2ⁿ−1 splice-variant space. If the statistical significance is high (p-value less than 0.05), it is considered as a reliable solution for prediction. Otherwise, it indicates the ECgene inclusive transcripts are not sufficient to represent the certain gene structure, in which case a particular set of criteria should be necessary for selecting splice variants. The information might be exon/intron length, exon presence frequency, or other types of characteristics such as motif, secondary structure, which may be relevant to alternative splicing mechanism and need more exploration.

This algorithm has been implemented as a computer program, in which each LP problem is solved using the LP solver provided in Matlib (Dantzig et al., 1999). The program uses an empirically determined cutoff to determine if a set of selected splicing isoforms gives close enough solution to the observed exon expression data. This program has been tested on a set of exon array data with experimentally validated splicing isoforms (Xi et al., 2008), where 17 splicing isoforms for 11 genes were confirmed using qRT-PCR. For these 11 genes, the solutions cover 81.8% of the experimentally verified splicing isoforms, indicating that the program is highly reliable.

Using this computational method, a total of 2,540 differentially expressed splicing isoforms (including full-length genes) have been identified between the 80 cancer tissues and 80 reference tissues collected. Simple validation experiments were performed on a few of the predicted splicing isoforms using PCR and isoform-specific primers (FIG. 1). For example, isoform-specific primers were prepared for three predicted splicing isoforms of the THY1 gene to check if any of the three predicted isoforms can be detected by the relevant primer. As shown in FIG. 1(c), splicing isoforms with identical masses to the three predicted isoforms were identified from the pool of expressed splicing isoforms of THY1.

In an alternative method, MIDAS (Affymetrix, 2005) was applied to the exon array data to detect if a gene has alternative splice variants. The basic idea is that under the null hypothesis of no alternative splicing for a gene, all exons in the gene should have statistically consistent expression levels. Then, the 1-way ANOVA method was used to test the null hypothesis through testing the constant effects model log(p_{i,j,k})=0 for all samples (0≦P_{i,j,k}≦1 is the proportionate expression of i-th exon of the j-th sample of k-th gene).

For each gene with splice variants determined above, the novel algorithm to predict the most probable set of splice variants was applied, along with a predicted expression level for each splice variant that is most consistent with the observed exon expression levels from the array data. Specifically, the algorithm first checks if the observed exon expression data for the gene can be well approximated using known splice variants of the gene in the ECgene database (Lee et al., 2007) along with an estimate for the most probable expression level for each variant. If the answer is yes, then the algorithm makes a prediction of a possible set of splice variants based on the ECgene database. Otherwise, the algorithm attempts to identify a minimal set of novel splice variants which, in conjunction with some of the known transcripts in ECgene, gives a good approximation to the observed exon expression data in the most parsimonious sense. This splice variant prediction problem is formulated as a linear programming (LP) problem, and solved using a public LP solver (Dantzig et al., 1999).

For each predicted set of splice variants, the following approach was used to assess its statistical significance. It was assumed, without loss of generality, that all the splice variants are from the ECgene database. For a gene consisting of n exons, let S be its predicted set of splice variants and v be the total difference between the observed expression value of each exon from the microarray data and the accumulated expression value across all the predicted splice variants along with their predicted expression levels across all n exons. The p-value of this predicted splice variant set, along with the expression levels, was assessed as follows. |S| splice variants were randomly selected from the corresponding gene entry in the ECgene database and assign a gene expression value for each splice variant so overall it gives the best fit for the observed exon expression value using the same procedure above. The difference for the above best fit is recorded as v′. This process was carried out for 10,000 times. If v is smaller than 95% of the v′ values, then the predicted S is accepted as reliable; otherwise, the prediction is rejected. Splice variant prediction was conducted using this approach on each gene deemed to have splice variants. The frequency of each predicted variant was then counted across all the 80 pairs of tissues. A splice variant was considered to be reliable if at least 30% of the tissues have this predicted variant.

### Example 5

### Differentially Expressed Genes in Gastric Cancer versus Reference Tissues

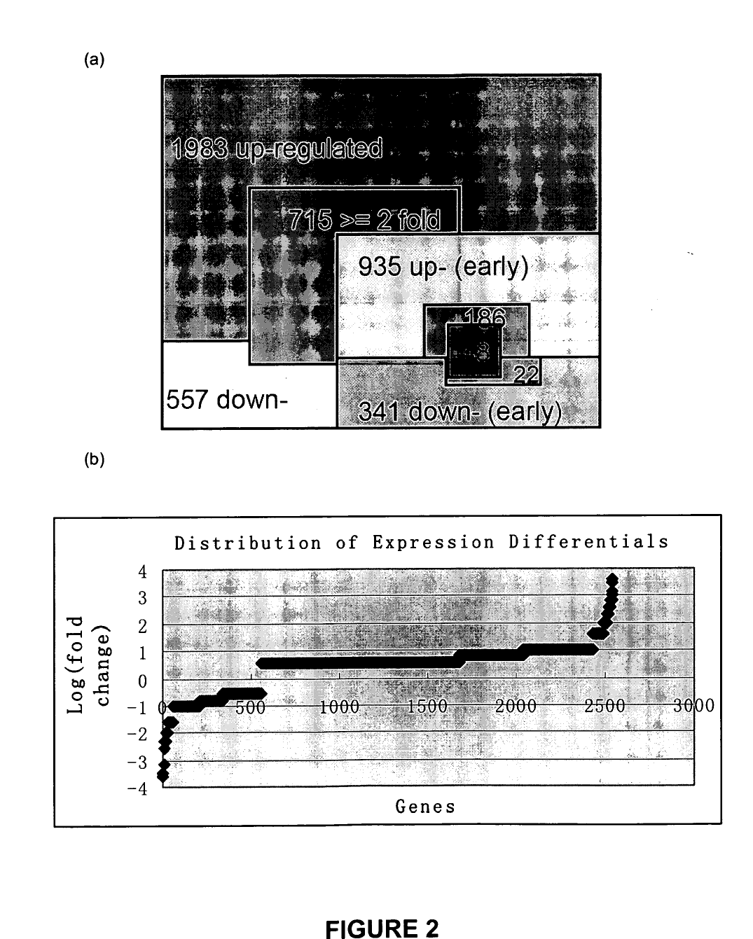

A total of 80 gastric cancer tissues and the same number of adjacent gastric but non-cancerous tissues from the same 80 patients were collected (see Table 2). Exon array experiments were conducted on these tissues using the Affymetrix GeneChip Human Exon 1.0 ST Array platform, which covers 17,800 human genes. Using a set of criteria discussed above, a total of 2,540 genes were found to exhibit differential expression patterns between the cancer and the reference tissues, of which 715 showed at least two-fold expression changes, as shown in FIG. 2(a). A gene refers to the collection of all its exons; it should be noted that the expression levels of individual exons may not necessarily be the same. A differentially expressed gene in cancer versus reference tissues refers to a gene with the summarized gene expression in cancer versus reference tissues being different. The majority of the 2,540 genes were up-regulated and one-fifth is down-regulated in cancer. In addition, 1,276 genes were differentially expressed in the early stage cancers (stages I and II), of which 935 were up-regulated and 341 were down-regulated. Among the 1,276 genes, 208 were differentially expressed across all early stage gastric cancer samples, with 186 up-regulated and 22 down-regulated, 48 of which are gastrointestinal diseases related (FIG. 2).

Of the 1,276 genes, 469 are differentially expressed only in early cancer tissues, i.e., having no substantial differences in advanced cancer tissues. The majority of the previously proposed marker genes are all up-regulated in cancer (Takeno et al., 2008). In contrast to the previous studies that were more focused on up-regulated genes, a large number of down-regulated genes were found in this study to be highly specific to gastric cancer. These include GIF, GNK1, GNK2, TFF1, GHL1, LIPF, and ATP4A, providing a different type of markers with decreased abundance in cancer.

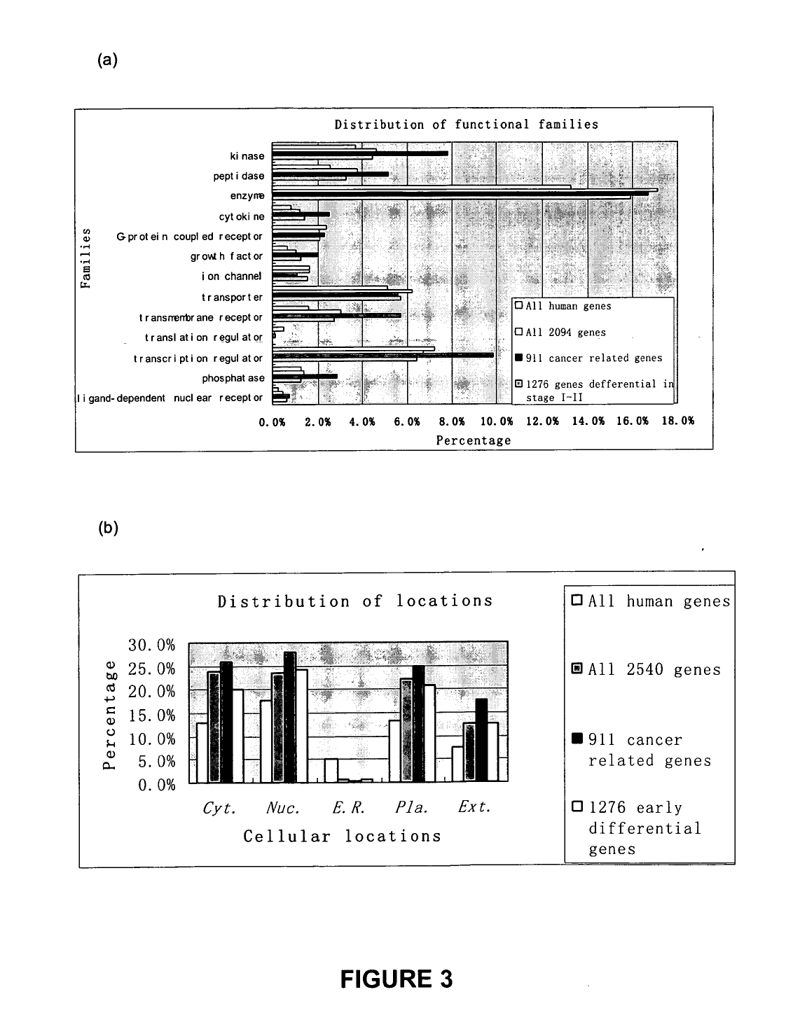

The functional families of the 2,540 genes, as defined by the Ingenuity Pathways Analysis (IPA) annotation were analyzed. Among them, 911 genes are cancer-related, 219 related to antigen presentation or immune responses, and 414 are gastrointestinal disease-related. Among the 13 major IPA functional families, 9 and 10 families were found to be substantially enriched among the 2,094 IPA-annotated genes (out of the 2,540) and the 911 cancer-related genes, respectively, when compared to the whole human gene set. As seen from FIG. 3(a), protein families such as kinases, peptidases, cytokines, growth factors, transmembrane receptors and transcription regulators are highly enriched in cancer-related genes, among which enzymes and transporters are more enriched in the differentially expressed genes. As seen from FIG. 3(b), the protein products of the 2,540 genes are generally localized in the cytoplasm, plasma membrane, extracellular space, or the nucleus. Similarly among the 468 genes differentially expressed only in early cancer tissues, 129 genes are cancer-related, 37 related to antigen presentation or immune responses, and 54 are gastrointestinal disease-related. Three functional families were found to be substantially enriched with these genes, namely enzymes, transcription regulators and transporters.

The differentially expressed genes found in this study have been compared with the gastric cancer-associated genes previously reported. Through an extensive literature search, 77 genes were found to be gastric cancer-associated and to have significantly differential expression during carcinogenesis and tumor progression (see Table 3). For 64 (83.1%) of the 77 genes, the expression data presented in this study are consistent with the previous findings, including genes such as TOP2A, CDK4, and CKS2 (El-Rifai et al., 2001), E-cadherin (Becker et al., 1994), GKN1, GKN2, and TFF1 (Hippo et al., 2002; Moss et al., 2008). For the other 13 genes the data presented in this study are novel. For example, genes related to chromosomal amplifications, transcriptional regulation, and signal transduction, such as cyclinE1, POP4, RMP, UQCRFS1 and DKFZP762D096, are found to have differential expression in 55 of the 80 (˜68.7%) cancer tissues in this study, compared to only ˜10% of 126 cancer tissues in a previous study (Chen et al., 2003). Another example is that up-regulation of the oncogene JUN (Dar et al., 2009) and down-regulation of the tumor suppressor gene, TP53 (Kim et al., 2007; Katayama et al., 2004) are found in no more than half of the patients analyzed in this study. One possible reason for these differences could be the different distributions of cancer stage, subtype, age, and gender of the samples used in this study versus the patient population in previous studies.

TABLE 3
Recent key findings of biomarkers by transcriptomic and proteomic studies on
gastric cancer

| | Genes | | Sample | |
|---|---|---|---|---|
| Reference | (findings) | Techniques | details | Category |
| Chen et al., | TSPAN1, | immunohistochemical | 86 cancer | cancer associated |
| 2008 | Ki67, CD34 | | tissues | genes |
| Long et al., | nuclear | immunohistochemical | 60 cancer | gene marker for |
| 2008 | factor kappa | | tissues | stage IV |
| Yamada et | PDCD6 | microarray analysis | 40 tissues + | prognostic gene |
| al., 2008 | | | 19 | biomarker |
| | | | independent | |
| Silva et al., | E-cadherin, | microarray + | 62 young + | gene markers |
| 2008 | beta-catenin, | immunohistochemistry | 453 old | |
| | and mucins | | patients | |
| | (MUC1, | | | |
| | MUC2, | | | |
| | MUC5AC | | | |
| | and MUC6) | | | |
| Xu et al., | MUC1 and | quantitative sandwich | 104 cancer | serum markers |
| 2009 | MUC5AC | enzyme immunoassay | and 120 | |
| | | | healthy | |
| | | | patients | |
| Takeno et | NEK6 and | microarray | 222 cancer | genes/proteins |
| al., 2008 | INHBA | | tissues | level |
| Kon et al., | pepsinogen | proteomics | gastric fluid | proteomic |
| 2008 | C, pepsin A | | from 24 | pattern |
| | | | cancer and | |
| | | | 29 benign | |
| | | | gastritides | |
| | | | patients | |
| Bernal et al., | reprimo | methylation-specific | 75 cancer | DNA |
| 2008 | | PCR | tissues, 43 | methylation |
| | | | cancer | patterns |
| | | | plasma and | |
| | | | 31 controls | |
| Taddei et al., | NF2 | RT-PCR | 5 | gene marker |
| 2008 | | | gastrointestinal | |
| | | | stromal | |
| | | | tumors | |
| Ebert et al., | cathepsin B | proteomics | epithelial | tumor cell/ |
| 2005 | | | cell and | serum marker |
| | | | serum | |
| Stefatic et | CEA, CA19- | — | — | serum markers |
| al., 2008 | 9, CA15-3, | | | review |
| | CA125, | | | |
| | ecPKA, | | | |
| | NNMT | | | |
| Jin et al., | MG7-Ag | ELISA | serum from | useful diagnosis |
| 2009 | | | 257 cancer + | makers |
| | | | 50 normal | |
| | | | patients | |
| Ren et al., | HSPB1, | SELDI-TOF-MS | serum from | protein pattern |
| 2006 | glucose- | | 46 cancer + | markers |
| | regulated | | 40 normal | |
| | protein, | | patients | |
| | PHB, PDIA3 | | | |

We have also identified a set of “marker” genes whose expression patterns can best distinguish between cancer and reference tissues using a combination of 1-, 2-, 3-, 4- and 5-genes. To do this, we have exhaustively searched through all k-gene combinations among the 2,540 genes, for 1<=k<=5, for the best markers between the cancer and the reference tissues, using a linear discriminate analysis in R (and validated using a linear SVM-based classification) on the computer clusters that our team has full access. The performance is evaluated by using the overall classification accuracy P=(TP+TN)/(TP+TN+FP+FN). Table 4 gives the top few k-gene markers for each k.

TABLE 4
Classification accuracy between cancer and reference samples using 1-, 2-,
3-, 4- and 5-gene markers, where accuracy is defined as the ratio between
the “true positive” and “true negative” predictions and the total
number of tissues.

| | | Accuracy |
|---|---|---|
| Gene markers | | (%) |
| 1 | TTYH3 | 80.1 |
| | LIPG | 78.7 |
| | MMP1 | 72.0 |
| 2 | LIPG-WNT2 | 83.9 |
| | LIPF-CD276 | 82.2 |
| | COL10A1-LIPG | 80.8 |
| 3 | AGTRL1-DPT-MMP1 | 89.7 |
| | TIMP2-DPT-COL10A1 | 89.1 |
| | DPT-THY1-LIPF | 88.4 |
| 4 | SLC5A5-ANGPTL3-MMP1-DPT | 93.1 |
| | COL10A1-LIPG-DTP-HOXB13 | 92.0 |
| | CLDN1-MMP1-SULT2A1-TRIM | 90.6 |
| 5 | COL10A1-LIPG-DTP-HOXB13- | 95.7 |
| | VIL1 | |
| | CLDN1-MMP1-SULT2A1-TRIM29- | 93.7 |
| | CDH17 | |
| | CLDN2-DPT-COL10A1-LIPG-DTP- | 92.7 |
| | HOXB13 | |

### Example 6

### Effects of Age and Gender on Gene Expression Data

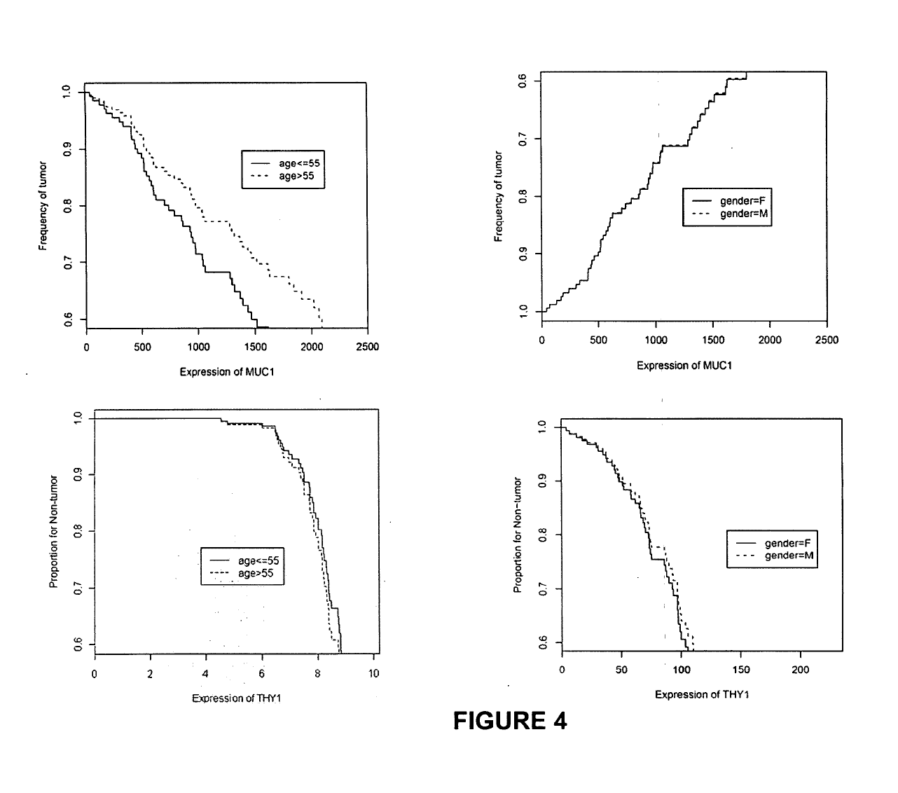

The impact of age and gender on the 2,540 differentially expressed genes have been assessed through multivariate analyses using ANOVA (Affymetrix, 2005) and the Cox Proportional Hazard Regress Model (Peduzzi et al., 1995). The key findings are summarized as follows (see Table 5 for detail). It was found that age significantly affects the expression levels of 143 of the 2,540 genes, most of which (113 out of 143) further increase the differences in their expression levels between the cancer and the reference tissues, an observation that could have important implications to biomarker selection. For example, it was found that the average MUC1 expression level is substantially higher among gastric cancer patients 55 years or older compared to patients younger than 55 (FIG. 4). Similar observations also hold for a few other genes such as the other members of the Mucin family, UBFD1, and MDK, while in contrast some other potential markers, e.g. THY1, are age-independent (FIG. 4).

TABLE 5
Statistics of multiple factors and their highly correlated genes identified by
ANOVA and Cox-proportional hazard regression analysis (p-value <0.05).

| | Genes highly correlated | |
|---|---|---|
| | # of | |
| Parameter | genes | Examples |
| Age | 143 | OLFM4, ABP1, DUOX2, TRIM31, GABRA3, |
| | | PRSS3, KRT17, GCNT3, LOXL2, TACSTD2 |
| Gender | 59 | SCNN1G, FGA, IL1A, CYP2B6, FAM19A4, |
| | | WNT2, ARSE, KCNN2, PCSK5, |
| | | TTLL6, HIST1H2BJ |
| Stage | 27 | MT1A, LIF, B3GNT6, HIST1H3J, MT1M |
| Smoking | 113 | TRIM29, PI3, FLJ42875, CKS2, DNER, |
| | | DUOX2, ANGPTL3, HRASLS2, PKM2, |
| | | DUOXA2, DSG3, APOBEC2 |
| Alcohol | 63 | KIAA1199, DSC3, COL11A1, C1orf125, |
| | | COL12A1, SULT1C2, LRRC15, SLCO1B3, |
| | | RPESP, GJB2, ADHFE1, RNF186, ANGPTL3, |
| | | ADRB2, APOBEC2, MT1L, PTK7, CKMT2 |
| Age + Gender | 118 | SDS, C1orf125, EGFL6, COL1A1, THY1, |
| | | REG4, ADH1A, CPS1, SORBS2, GPR68, |
| | | TIMP1, ADH1C |
| Age + Stage | 379 | ALDH3A1, GSTM5, SORBS2, ADH1A, |
| | | CDH13, RASL12, GPM6B, PCOLCE2, |
| | | CAB39L, CASQ2, ACADL, MAMDC2, |
| | | ZBTB16, C8orf42, MT1A, ADAMTSL3, |
| | | CNTN1, GPX3 |

Possible gender-specific biases in the expression data presented were also examined, knowing that the male-to-female ratio of gastric cancer occurrences is about 2:1 (Chandanos and Lagergen, 2008). It was found that the expression levels of 59 genes, such as WNT2, ARSE, and KCNN2, are gender-dependent (see Table 5 for the complete list). An interesting observation is that the combination of age and gender has a more significant effect on gene expression levels of 118 genes including COL1A1, THY1, REG4, ADH1A, and CPS1. For genes like TIMP1 and ADH1A, older male patients have higher expression levels than younger female patients. It was also found, among the differentially expressed genes unique to early cancers, 28 and 9 genes are age- and gender-dependant, respectively, from which genes like P2RY6 and NSUN5 belong to both groups.

### Example 7

### Co-expressed Genes and Enriched Pathways in Cancer Tissues

With the goal of discovering novel associations of genes with specific subtypes and developmental stages of gastric cancer, the gene expression data was analyzed using a bi-clustering analysis. The bi-clustering program QUBIC (Li et al., 2009) was used for this study. The basic idea of the algorithm is to find all subgroups of genes with similar (or co-related) expression patterns among some (to be identified) subset of cancer tissues. The QUBIC program is unique in its ability to detect complex relationships (beyond just sharing similar expression patterns), and to do so in a very efficient manner even for datasets containing tens of thousands of genes and thousands of tissue samples. The algorithm is presented in detail in Li et al., 2009.

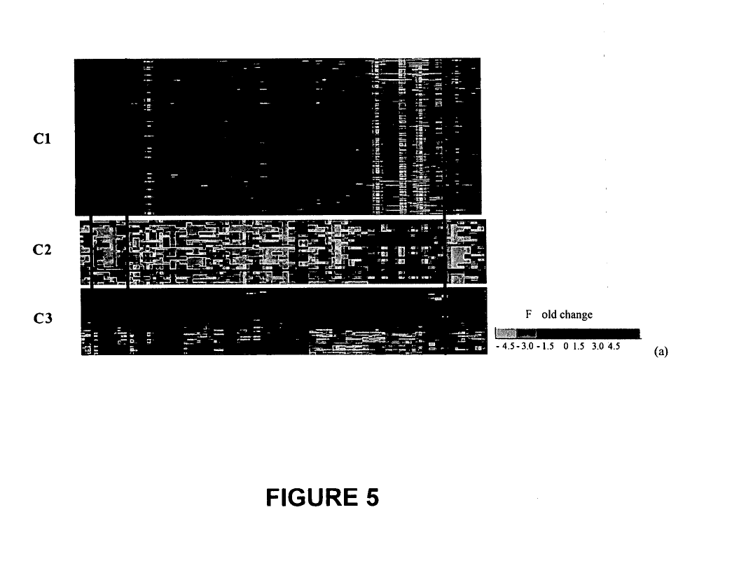

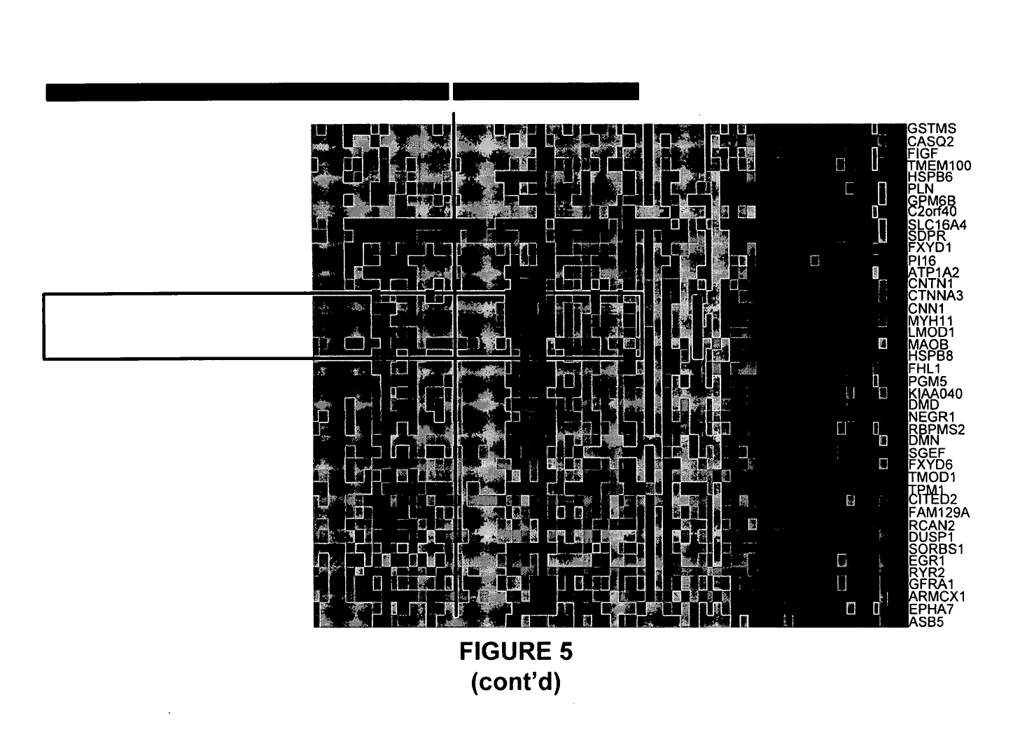

Utilizing the bi-clustering program QUBIC, 14 statistically significant bi-clusters have been identified and analyzed, which are cancer specific, stage-, subtype- or gender-specific. Three identified bi-clusters, C1, C2, and C3 are first highlighted. FIG. 5(a) summarizes the genes in C1 and C2 and their associated expression patterns across the majority of all the 80 cancer-reference tissue pairs, particularly across all tissue pairs in early stage cancers.

Detailed analyses of these two bi-clusters (C1 and C2) revealed that (a) genes such as transcriptional regulators, growth factors, and enzymes involved in cell cycle (STMN1 and CDCA8), transcription regulation (TCF19 and BRIP1), angiogenesis (IL8), chromosome integrity (TOP2A), and extracellular matrix remodeling (MMPs) were activated at a very early stage of gastric cancer (in C1), while genes involved in metabolism are de-activated (in C2); and (b) most genes in C1 and C2 show discerning power between cancer and reference tissues even at stage I. Examples include HOXB13, TOP2A, CDC6, and CLDN7 being up-regulated across all early stage cancers and ˜80% of all cancer tissues, and CHIA being down-regulated across all early stage cancers and 79.1% of all cancer tissues. Some of the C3 genes exhibit different expression patterns unique to specific cancer stages. For example, SPP1, SPRP4, COLBA1, INHBA, CTHRC1, COL1A1, THBS2, SULF1, and COL12A1 are over-expressed across most of the stages III and IV cancer tissues while no consistent patterns are observed in stages I and II cancer tissues (FIG. 5). This group of genes can provide potential markers for measuring the progression of gastric cancer.

Another identified bi-cluster provides useful information about subtypes as shown in FIG. 5(b), in which the 80 patients are partitioned into two distinct groups (the green part on the left and the red part on the right), which are unrelated to stages. This bi-cluster consists of 42 genes and 80 patients. Six of the 42 genes, namely CNN1, MYH11, LMOD1, MAOB, HSPB8, and FHL1, have been previously reported to be differentially expressed between the intestinal and the diffuse subtypes of gastric cancer (Kim et al., 2007). This seems to indicate that these 42 genes can distinguish two possible subtypes of gastric cancer.

### Example 8

### Pathway Enrichment Analysis

Pathways enriched by the differentially expressed genes have also been examined. The pathway enrichment analysis for a given set of genes was done using two programs, DAVID (Dennis et al., 2003) and KOBAS (Wu et al., 2006). DAVID computes an EASE score (a modified Fisher Exact P-value) to evaluate the enrichment ratio of relevant pathways, based on GO Biological Processes and BIOCARTA pathways, while KOBAS computes four statistical scores to assess enriched pathways, using all KEGG pathways and KEGG Orthology (KO). Besides these sources, information was integrated from the UCSC Cancer pathway database (Zhu et al., 2009) which includes a human Pathway Interaction Database curated by NCI-Nature (Schaefer et al., 2009). Then the modified p-value was calculated for each enriched pathway based on Fisher's exact test on queried genes against all genes in human genome. Table 6 lists 13 such pathways.

TABLE 6
Thirteen enriched pathways by differentially expressed genes. ↑ for
up- and ↓ for down-regulation. P-value is calculated for a pathway
enriched in all stages except those marked with * are for early stage only.

| | # of genes | | |
|---|---|---|---|
| | Stages I-II | All | |
| Pathways | (specific) | stages | P-value |
| Cell cycle | 22↑ (9↑) | 49↑ | 1.59E−21 |
| p53 signaling pathway | 10↑ (3↑) | 27↑ | 2.66E−12 |
| ECM-receptor interaction | 4↑ (—) | 31↑ | 8.18E−13 |
| Cell communication | 6↑ (—) | 34↑ | 4.70E−04 |
| Cell adhesion molecules (CAMs) | 4↑ (2↑) | 31↑ | 5.13E−04 |
| Role of BRCA1, BRCA2 and ATR in | 4↑ (—) | 10↑ | 2.90E−03 |
| cancer susceptibility | | | |
| E2F1 destruction pathway | 4↑ (—) | 6↑ | 8.00E−03 |
| Wnt signaling pathway | 4↑ (—) | 17↑ | 2.22E−02 |
| Focal adhesion | 4↑ (3↑) | 41↑ | 1.32E−09 |
| | 3↓ (3↓) | 4↓ | 9.81E−02* |
| Metabolism of xenobiotics by | 4↓ (—) | 16↓ | 7.21E−04* |
| cytochrome P450 | | | |
| Arginine and proline metabolism | 3 ↓ (—) | 3↓ | 1.16E−03* |
| Fatty acid metabolism | 3↓ (—) | 7↓ | 2.56E−03* |
| Insulin signaling pathway | 5↓ (—) | 7↓ | 9.37E−04* |

It can be seen from Table 6 that genes involved in cellular proliferation, cell cycle, and DNA replication were consistently up-regulated across the majority of the cancer samples, while those involved in fatty acid metabolism, digestion, and ion transport were consistently down-regulated. Most of these pathways start being up-/down-regulated in early stage cancers and become highly enriched in advanced cancers. Besides the general cancer-related pathways such as cell cycle and regulation, DNA damage and repair, cell growth, death and regulation, and estrogen receptor regulation pathways, some gastric cancer-specific processes were also revealed. For example, a novel thyroid hormone mediated gastric carcinogenic signaling pathway is enriched with up-regulated genes (TTHY, PKM2, GRP78, FUMH, ALDOA, and LDHA) in cancer tissues (Liu et al., 2009), most of which are in advanced stages. Another interesting observation is that certain pathways are only and more enriched in tissue samples of either male or female. For example, role of Ran in mitotic spindle regulation, Wnt signaling pathway and Bisphenol A degradation are enriched in male but not in female, while Ghrelin, 3-chloroacrylic acid degradation, alternative complement pathway and histidine/tyrosine/nitrogen/cysteine metabolisms are more enriched in female. These findings could provide new angles to study gastric cancer formation and progression.

### Example 9

### Alternative Splice Variants of Genes in Cancer versus Reference Tissues

A signature selection procedure was used to identify multi-gene markers that can distinguish between the cancer and the reference tissues based on random sampling and a multistep evaluation of the gene-ranking consistency (Bell et al., 1991). The basic idea is as follows: an SVM-based recursive feature elimination (RFE) approach was employed to find the minimum subsets of genes (features) that obtain the best classification performance of 500 trained SVMs on 500 equal-sized subsets of randomly selected samples. Gene(s) are eliminated if they meet two criteria: (1) more than 80% of the 500 classifiers consistently rank them as the 10% least important genes for our classification; and (2) they have never been ranked within the top 50% in (1). This gene-selection process continues until the remaining set of genes cannot be further reduced without going below a pre-defined cutoff for classification accuracy.

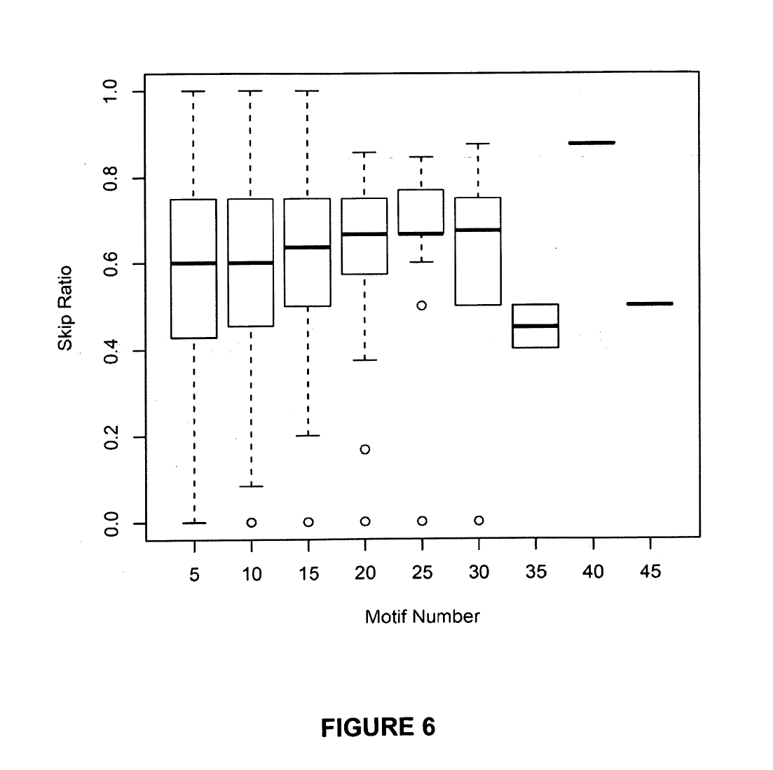

Among the 2,540 differentially expressed genes, 1,875 are identified to have alternative splice variants by a novel algorithm as discussed in Example 4 above. 69.2% and 72.8% of the 1,875 genes in the reference and cancer tissues, respectively, have substantial splicing structure changes based on the prediction. Out of the 1,875 genes, it was predicted 11,757 different splice variants in total, among which 6,532 and 6,827 are present in more than 30% of the cancer and reference tissues, respectively, which are considered as reliable predictions. While splice variants below this cutoff could also be true, such data become less reliable and more challenging to interpret. Hence splice variants below this cutoff were not considered further in this study. 6,114 of the splice variants appear in both cancer and reference tissues, out of which 3,933 are differentially expressed in the gastric cancer versus the reference tissues, and 94 are differentially expressed only in early gastric cancer. The predicted exon-skipping events in these predicted splice variants have been checked, and it has been found that the more frequently skipped exons in the predicted alternative splice variants tend to be associated with intronic regions having more cis regulatory motifs for splice regulation, consistent with the previous observation (Wang et al., 2008) as shown in FIG. 6, providing one supporting evidence for the predicted splice variants although substantial experiments are needed to validate all the predicted splice variants.

Such analysis of the splice variants revealed that (a) a total of 4,733 novel splice variants are predicted by comparing them with known transcripts in the Ensemble database (Eyras et al., 2004), the most comprehensive database for splice variants for human; (b) genes with the most differentially expressed splice variants are cancer related, including COL11A1, CTSC, CDH11, and WNT5A; (c) the number of different splice variants increases as the cancer progresses from stage I to stage IV; and (d) 1,690 and 1,377 splice variants unique to female and male patients, respectively, were found; and 364 and 126 of those are differentially expressed in cancer versus reference tissues, respectively.

Among the early stage cancer-specific splice variants, 84 of their parent genes are involved in such pathways as tight junction, calcium signaling, pyrimidine metabolism, Wnt signaling and epithelial cell signaling known to be associated with Helicobacter pylori infection (Kanehisa and Kegg, 2000). In addition, among all the differentially expressed splice variants, their parent genes include the members of the Wnt pathway (CTNNB1, WNT2, SFRP4, WISP1, WNT5A), integrin signaling (ITGAX), p53 signaling (E2F1, CDK2, PCNA, TP53, BAX, CDK4), and extracellular matrix proteins (FN1, COL6A3), and other genes such as VEGFC, FGFR4, CEACAM6, CDH3, NCAM1, MSH2, VCL, and ANLN. It was also noticed that 10 transcription factors have expressed splice variants, although not in early stage, namely TFAP2A, NOC2L, MYBL2, MSC, HOXA13, H2AFY, ETV4, E2F4, CCNA1, and BRD8, which could serve as important indicators for cell growth and survival, proliferation, differentiation or apoptosis.

### Example 10

### Signature Genes for Gastric Cancer and Stages

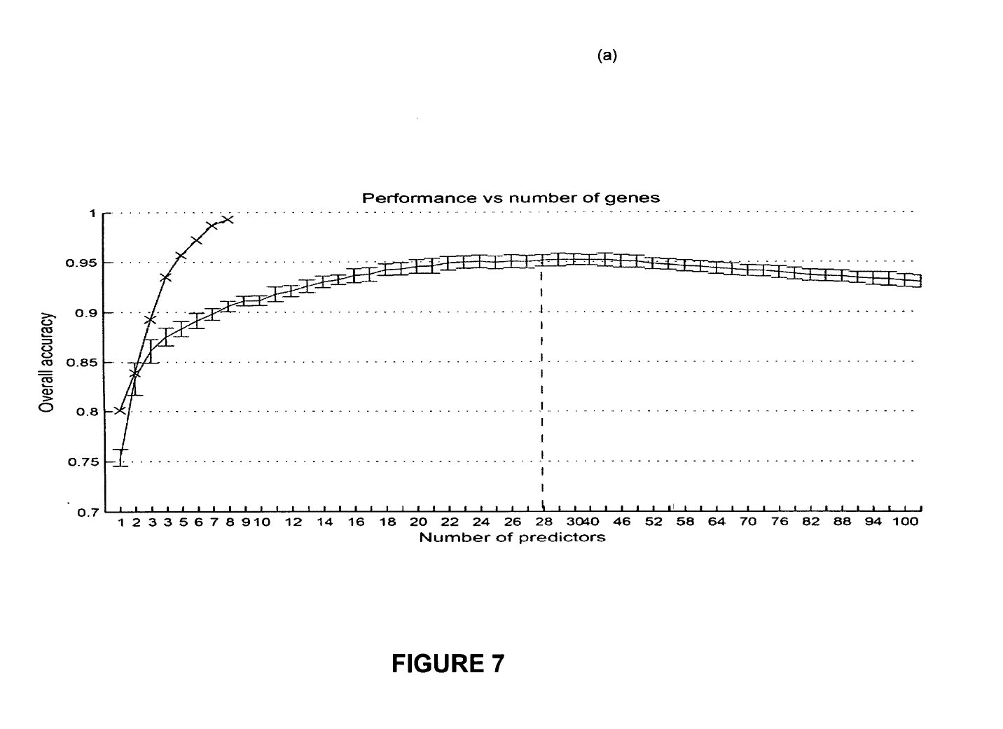

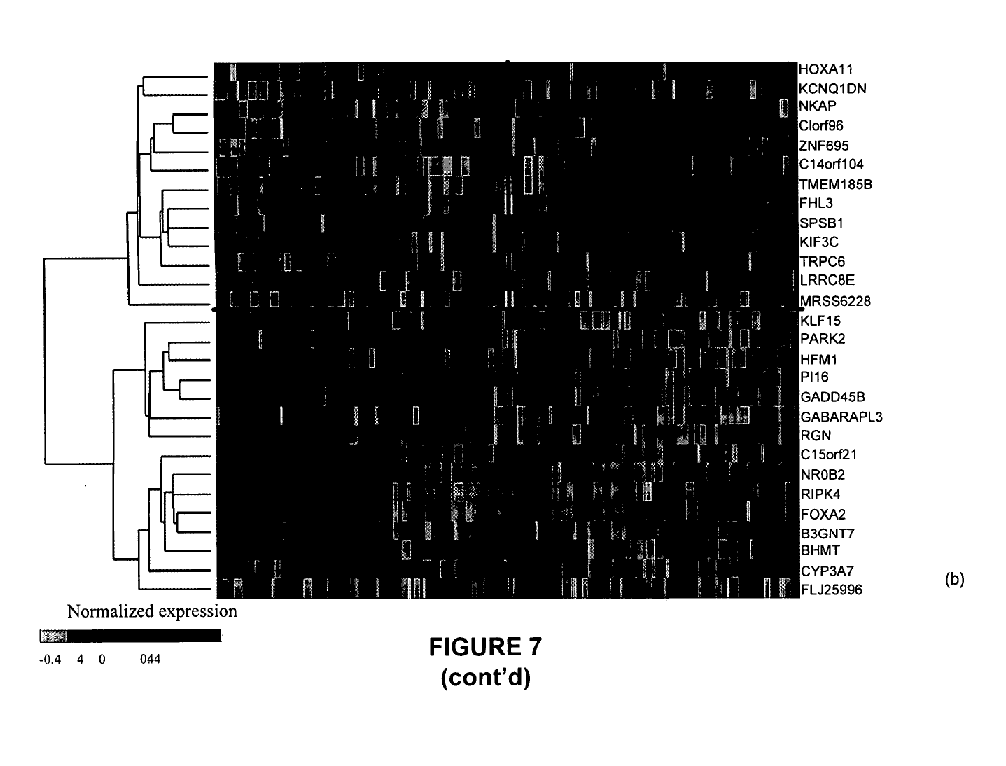

As discussed in Example 9 above, a number of genes have been identified whose expression patterns can well distinguish the cancer from the reference tissues by using an efficient RFE-SVM method. FIG. 7(a) summarizes the classification accuracies for the selected optimal k-gene markers for k from 1 to 100. It can be seen from the figure that the 28-gene marker group is the best across all k's, having 95.9% and 97.9% agreement with the cancer and reference tissues, respectively (see Table 7 for their gene names).

The design of the RFE-SVM-based procedure took into consideration of classification accuracy, stability and reproducibility, and hence the results are highly generalizeable. An exhaustive search has also been carried out for the best k-gene marker groups by going through all k-gene combinations, which guarantees to find the globally optimal markers at the expense of losing the computational efficiency of the RFE-SVM method for all k<=8, using a linear SVM approach (Vapnik, 1995). The performance of the identified k-gene markers is evaluated using both leave-one-out and five-cross validation methods. As shown in FIG. 7(a), the best accuracies of the so identified k-gene markers (k=1 . . . 8) are consistently better than those by the RFE-SVM method. This analysis indicates that these best marker genes are associated with the following known pathways: cell cycle, ECM-receptor interaction, CDK regulation of DNA replication, and the TNFR1 signaling pathway (see Table 7 for detail).

An interesting observation is that some markers perform very well for certain groups of patients, but not for other groups such as for patients of different genders and ages. This is consistent with observations presented in Example 6 above, that age and gender have considerable effects on gene expression levels. To overcome this problem, a marker search for different genders separately has been conducted. The detailed list of the markers for the two gender groups are given in Table 7, which lists the top gender-specific markers including LIPG, INHBA, MFAP2 and TTYH3 for female and WNT2, CD276 and MFAP2 for male.

A similar analysis on the early stage cancer samples (stages I and II) was also carried out, and a number of promising markers unique to early stage gastric cancer were identified. For example, genes such as HOXB9, HIST1H3F, TMEM25, and CLDN3 consistently show differential expressions across all early stage cancer tissues, but no similar differential expressions were observed in advanced cancers. Table 7 gives the best k-gene marker groups along with their classification accuracies for the early cancers. Overall, it was found that the best single-gene marker can obtain up to 94.4% classification agreement with 100% for cancer and 88.9% for reference tissues, respectively. This number improves to 97.3% when using the best 2-gene markers.

To examine the generality of the predicted gene markers, their classification accuracies have been checked on previously published large microarray datasets for gastric cancer by other groups. On the GSE2701 dataset by Xin et al., 2003, the success rates of the k-gene markers of this study range from 81.7% to 100% when k goes from 1 to 7. When evaluated on the early stage samples from the Kim dataset (Kim et al., 2007), the single-gene markers of this study such as TFF3, CLDN4, MDK, and MUC13 show consistent differential expression patterns across 80% (12 of 15) of their early stage samples. Overall these results indicate that the identified tissue markers are generally applicable.

The splice variants of the predicted gene markers have been examined and a number of splice variants as possible markers have been predicted based on the identified gene markers and their predicted splice variants, either over- or under-expressed in cancer versus reference tissues. While the detailed results are given in Table 7, a few splice-variant markers are listed here: over-expressed splice variants LMNB2:000111111111, WNT2:11111, WNT2:00111, LIPG:1111111110 and LIPG:1111110000, and under-expressed splice variants AQP4:111110, GRIA4:0001111110000000 and ESRRG:0111110110000000, where “1” in the i-th position represents the presence of the i-th exon of the gene in the splice variant and “0” indicates its absence.

TABLE 7
Detection accuracies of top five 1-, 2-, 3- and 4-gene markers predicted for
different categories, including general markers, early-stage specific and
gender-specific markers. Accuracy (Acc.) is measured as the mean of
100 times 5-cross-validation (CV) detection accuracies.
Detection accuracies of predicted markers (5-CV)

| | | | Early | | | | | |
|---|---|---|---|---|---|---|---|---|
| | General | | stage I-II | | | | Female | |
| | markers | Acc. | only | Acc. | Male only | Acc. | only | Acc. |
| 1 | CD276 | 80.1 | HIST1H3F | 94.4 | WNT2 | 79.8 | LIPG | 91.3 |
| | TTYH3 | 80.1 | CCL20 | 94.4 | CD276 | 78.7 | INHBA | 86.9 |
| | LIPG | 78.7 | HIST1H3F | 94.4 | MFAP2 | 77.7 | MFAP2 | 86.9 |
| | LMNB2 | 78.7 | C2orf40* | 94.4 | TTYH3 | 77.7 | TTYH3 | 86.9 |
| | WNT2 | 78.1 | HOXB13 | 88.9 | PON2 | 76.6 | RUNX1 | 86.9 |
| | COL1A1 | 77.4 | CLDN3 | 88.9 | HOXB9 | 75.5 | GPER* | 86.9 |
| | PON2 | 77.4 | HOXB9 | 88.9 | CDH3 | 75.5 | GKN1* | 86.9 |
| 2 | CST1-ITGB8 | 81.5 | SCN7A- | 94.4 | MYOC- | 90.4 | INTU-LIPG | 97.8 |
| | | | IKIP | | BHLHB2 | | | |
| | CST1-AGT | 81.5 | HIST1H4I- | 94.4 | DPT- | 88.3 | C16orf53- | 97.8 |
| | | | TFCP2L1 | | VASH1 | | LIPG | |
| | MMP1- | 80.8 | FAM129A- | 94.4 | MAMDC2- | 87.2 | Gcom1- | 97.8 |
| | INHBA | | TREM1 | | MMP2 | | GPRIN3 | |
| | MMP1- | 80.1 | MYO1B- | 94.4 | CFD- | 86.2 | CST7-LIPG | 95.6 |
| | COL1A1 | | MYH11 | | THY1 | | | |
| | LIPG-WNT2 | 83.9 | WNT3- | 94.4 | DGKB- | 86.2 | CRABP2- | 95.6 |
| | | | NUDCD1 | | WNT2 | | UCKL1 | |
| | LIPF-CD276 | 82.2 | TMEM25- | 94.4 | C2orf40- | 85.1 | HOXB9- | 95.6 |
| | | | HOXB5 | | PLXDC1 | | LIPG | |
| | COL10A1- | 80.8 | MMP1- | 88.9 | DPT- | 85.1 | CLDN1- | 95.6 |
| | LIPG | | MFAP2 | | COL1A1 | | LIPG | |
| 3 | AGTRL1- | 89.7 | SCN7A- | 94.4 | CD44- | 93.6 | GIF*- | 100 |
| | DPT-MMP1 | | IKIP- | | DPT- | | PID1- | |
| | | | HIST1H3F | | AGTRL1 | | LRRIQ1 | |
| | TIMP2-DPT- | 89.1 | SCN7A- | 94.4 | GGTLA1- | 92.5 | FCGR3A- | 100 |
| | COL10A1 | | IKIP- | | DPT- | | C16orf53- | |
| | | | C2orf40 | | NID1 | | LIPG | |
| | DPT-THY1- | 88.4 | HIST1H4I- | 94.4 | LOC202051- | 92.5 | SLC15A3- | 100 |
| | LIPF | | TFCP2L1 | | CGNL1- | | PAICS- | |
| | | | | | THY1 | | FAM123A | |
| | THBS2- | 88.4 | SCN7A- | 88.9 | FRMD1- | 92.5 | SLC15A3- | 97.8 |
| | DPT- | | IKIP | | MAMDC2- | | LIPG- | |
| | C19orf40 | | RYR2 | | RASAL2 | | TPD52 | |
| | TIMP2-DPT- | 88.4 | SCN7A- | 88.9 | HOXB9- | 91.5 | SLC15A3- | 95.7 |
| | CLIC1 | | IKIP- | | RYR2- | | LIPG- | |
| | | | C2orf40 | | CD109 | | SPON2 | |
| | MYOC- | 88.4 | SCN7A- | 88.9 | PDZRN4- | 91.5 | SLC15A3- | 95.7 |
| | CD44- | | IKIP- | | INHBA- | | MYOC- | |
| | HIST2H2AB | | CCL20 | | AGTRL1 | | CD3EAP | |
| 4 | CXorf36- | 94.5 | GAL3ST4- | 94.4 | RYR2- | 95.7 | EPDR1- | 100 |
| | DPT-CD44- | | PPA1- | | HMCN1- | | GIF*- | |
| | BST2 | | HOXA13- | | HOXB9- | | TEAD4- | |
| | | | HIST1H3F | | MT1M | | OR1L1 | |
| | PDGFRB- | 93.8 | — | — | TGM2- | 95.7 | KIAA1199- | 100 |
| | MYOC- | | | | PARK2- | | DUSP10- | |
| | HFM1- | | | | RASGRF | | LYCAT- | |
| | PGRMC2 | | | | 2-PI16 | | ADHFE1 | |
| | SLC5A5- | 93.1 | — | — | MEX3D- | 95.7 | FCGR3A- | 100 |
| | ANGPTL3- | | | | DPT- | | PGRMC2- | |
| | MMP1-DPT | | | | C10orf72- | | GLIS3- | |
| | | | | | C10orf129 | | TMEM40 | |
| | COL10A1- | 92.0 | — | — | NR0B2- | 95.7 | CKMT2- | 100 |
| | LIPG-DTP- | | | | BTG2- | | CCL18- | |
| | HOXB13 | | | | CTSA- | | MICALL1- | |
| | | | | | DBT | | LRRIQ1 | |
| | CLDN1- | 90.6 | — | — | IRX3- | 95.7 | PTGIR- | 100 |
| | MMP1- | | | | ADCYAP1R1- | | GAL3ST4- | |
| | SULT2A1- | | | | FADS2- | | PTPRS- | |
| | TRIM | | | | RUNX1 | | XAF1 | |

(gene marked with * are those down-regulated in cancer versus reference “—”: k-gene markers were omitted here if combination markers with smaller k already have 100% or unchanged best detection accuracy or on our samples)

### Example 11

### Development of a Computational Method for Prediction of Blood-Secretory Proteins

A computational technique has been developed for predicting human proteins that can be secreted into circulation (Cui et al., 2008). The basic idea of the method is to collect a set of known blood-secreted proteins and a set of proteins that are not homologous to any proteins that have been detected in human sera. Then a classifier is trained to distinguish between the two sets. A large number of features computable from protein sequences have been examined and the features that can provide the highest discerning power between the two sets have been identified.

The starting point for collecting the training data is the dataset containing ˜16,000 proteins that have been detected in human sera, compiled by the Plasma Proteome Project (PPP) (Omenn et al., 2005). 1,620 human secreted proteins from the Swissprot and the SPD database (Chen et al., 2005) were also collected. By comparing this list against PPP, 305 proteins, belonging to both sets, were found that are not among the native blood proteins. Hence, these 305 proteins are considered as being secreted into blood and were used as the positive set. Representatives were then selected from each family of Pfam (Bateman et al., 2002) that does not overlap with PPP, and 26,962 proteins were collected as the negative set. The positive and the negative sets were then split into training and testing sets.

To find features that can distinguish the two sets, over 50 features were examined that fall roughly into four categories: (i) general sequence features such as amino acid composition and di-peptide composition (Reczko et al., 1994; Bhasin et al., 2004); (ii) physicochemical properties such as solubility, disordered regions and charges, (iii) structural properties such as secondary structural content and solvent accessibility, and (iv) specific domains/motifs such as signal peptides, transmembrane regions and the twin-arginine signal peptide motif (TAT).

Using these features, a support vector machine (SVM)-based classifier was trained to distinguish the positive from the negative training data using a Gaussian kernel (Platt et al., 1999; Keerthi et al., 2001). Based on the performance of the initial SVM, a feature-selection procedure, called recursive feature elimination (RFE), was employed to remove features irrelevant or negligible to the classification goal. The feature selection process iteratively removes irrelevant features based on a consensus scoring scheme and gene-ranking consistency evaluation (Tang et al., 2007). Specifically, in each iteration, features with the lowest scores (lowest ranked) given by RFE are eliminated from the feature list. This process continues until a minimal set of features is obtained while maintaining the level of classification performance. Throughout the training, random sampling (Bell et al., 1991) has been employed to generate the training and testing sets, and a classifier has been trained based on the given training and testing sets. This process was performed 500 times and the most representative one was picked (Cui et al., 2008) as the selected one. After this process, the most important features for the classification were found to include transmembrane regions, charges, TatP motif, solubility, signal peptides, and O-linked glycosylation motif.

Based on the selected features, an SVM-based classifier has been retained, cross-validated and its performance tested on an independent evaluation set, which can correctly classify 90% of the blood-secreted proteins and 98% of non-blood-secreted proteins. Several additional datasets are used to further assess the performance of the classifier, each of which contains recently identified blood-secreted proteins and those reported in the literature. The test results give comparable performance statistics with the ones on the evaluation set. For example, a list of 122 proteins detected in human sera by mass spectrometry was compiled through an extensive literature search. These proteins are overly expressed in at least one of 14 types of human cancers, and none of them is included in our training set. 97 out of 122 (79.5%) proteins were predicted correctly using the method described above.

### Example 12

### Prediction of Blood-Secreted Proteins

Among all differentially expressed genes, those that can be secreted into the bloodstream as possible serum markers were focused on. A computational method has been developed for prediction of such secreted proteins (Cui et al., 2008). This example describes an approach for predicting secretion of proteins into serum. However, based on the teaching and guidance presented herein, it is understood that it is known in the art to readily adapt the methods described herein to predict secretion of proteins into other biological fluids, such as, but not limited to, saliva, spinal fluid, seminal fluid, vaginal fluid, amniotic fluid, gingival crevicular fluid, and ocular fluid.

A number of serum protein markers for gastric cancer have been predicted based on their identified differential expressions in cancer tissues and the blood secretion prediction (Cui et al., 2008). These predicted serum markers are grouped into three categories: (a) general markers for gastric cancer, (b) markers specific to early stage cancer, and (c) gender-specific markers. Table 8 shows the proteins that are considered as the most promising either individually or combined as groups. Detailed information about these and other promising marker proteins is given in Table 9.

Among these predicted serum markers, MMP1, MUC13, and CTSB are effective gene discriminators between cancer and reference tissues, but they are not specific for gastric cancer because of their over-expression in other cancers such as breast, ovarian, lung and colon cancer (Poola et al., 2008). LIPF, GAST, GIF, GHRL and GKN2 are, however, gastric tissue specific, thus making them promising serum markers for gastric cancer, particularly when used in conjunction with other markers.

TABLE 8
Examples of the most promising predictive markers for gastric cancer

| | | Stage efficiency | | Gender specificity | |
|---|---|---|---|---|---|
| Serum Marker | | General | Early | Female | Male |
| MMP1 | Matrix metalloproteinase | ✓ | | | |
| | 1 preproprotein | | | | |
| MUC13 | Mucin-13 | ✓ | | | |
| CTSB | Cathepsin B | ✓ | | ✓ | |
| GKN2 | Gastrokine-2 | | ✓ | ✓ | |
| GHRL | Appetite-regulating | | ✓ | | |
| | hormone (Ghrelin) | | | | |
| LIPF | Gastric triacylglycerol | | ✓ | ✓ | |
| | lipase (gastric lipase) | | | | |
| LIPG | Endothelial lipase | ✓ | | ✓ | |
| LIMK1 | LIM domain kinase 1 | | ✓ | † | † |
| GAST | Gastrin | | ✓ | | |
| GIF | Gastric intrinsic factor | ✓ | | | |
| AZGP1 | Zinc-alpha-2- | ✓ | | | |
| | glycoprotein | | | | |

(† indicates that a gene has good classification accuracy but is gender-independent)

TABLE 9
Detailed information of 18 predictive markers, along with their functional
annotation, expression specificity in cancers, and related diseases.

| | | | | Subcellular | | | |
|---|---|---|---|---|---|---|---|
| | | | | location & | | Reported | |
| | | | | Presence in | | expression | |
| | | | | blood | | in cancers | |
| Gene | Protein | Mass | | (annotation*/our | | (versus | Relevant |
| symbol | [AC] | (kDa) | FC | prediction) | AS | normal) | diseases |
| MMP1 | Matrix | 44.8 | 7 | extracellular | ✓ | breast; | cancer, |
| | metalloproteinase 1 | | | Space & (1/1) | | colon; | cardiovascular |
| | preproprotein | | | | | tongue; | disease, |
| | [Q53G97] | | | | | moderately | hepatic |
| | | | | | | over- | system |
| | | | | | | expressed | disease, |
| | | | | | | in head & | inflammatory |
| | | | | | | neck; lung; | disease, |
| | | | | | | bladder | neurological |
| | | | | | | cancer | disease |
| COL10A1 | collagen | 66.2 | 3 | secreted; | | colon; | connective |
| | alpha-1(X) | | | extracellular | | breast | tissue |
| | chain | | | matrix & (1/1) | | cancer | disorders, |
| | [Q03692] | | | | | | dermatological |
| | | | | | | | diseases, |
| | | | | | | | inflammatory |
| | | | | | | | disease, |
| | | | | | | | skeletal and |
| | | | | | | | muscular |
| | | | | | | | disorders |
| CLDN1 | claudin-1 | 22.7 | 4 | plasma | ✓ | moderately | cancer, |
| | [O95832] | | | membrane & | | over- | dermatological |
| | | | | (0/1) | | expressed | diseases |
| | | | | | | in | and |
| | | | | | | seminoma | conditions, |
| | | | | | | and ovarian | gastrointestinal |
| | | | | | | cancer | disease |
| TOP2A | DNA | 174.4 | 3 | cytoplasm; | ✓ | bladder; | antigen |
| | topoisomerase | | | nucleus & | | brain; liver | presentation, |
| | 2-alpha | | | (1/0) | | cancer | cancer, |
| | EC = 5.99.1.3 | | | | | | dermatological |
| | [P11388] | | | | | | diseases |
| | | | | | | | and |
| | | | | | | | conditions, |
| | | | | | | | gastrointestinal |
| | | | | | | | disease |
| CST1 | cystatin-SN | 16.4 | 12 | secreted & | | moderately | cancer, |
| | precursor | | | (0/1) | | over- | neurological |
| | [P01037] | | | | | expressed | disease |
| | | | | | | in bladder; | |
| | | | | | | head-neck; | |
| | | | | | | seminoma | |
| COL1A1 | collagen | 138.9 | 3 | extracellular | ✓ | seminoma; | antigen |
| | alpha-1(I) | | | space & (1/1) | | moderately | presentation, |
| | chain | | | | | over- | auditory |
| | [P02452] | | | | | expressed | disease, |
| | | | | | | in brain; | cancer, |
| | | | | | | head & | cardiovascular |
| | | | | | | neck; | disease, |
| | | | | | | gastric | connective |
| | | | | | | cancer | tissue |
| | | | | | | | disorders, |
| | | | | | | | hepatic |
| | | | | | | | system |
| | | | | | | | disease, |
| | | | | | | | inflammatory |
| | | | | | | | response |
| MUC13 | Mucin-13 | 54.6 | 2 | secreted & | | highly | cancer, |
| | [Q9H3R2] | | | (1/1) | | expressed | gastrointestinal |
| | | | | | | in | disease |
| | | | | | | epithelial | |
| | | | | | | cancer | |
| | | | | | | tissues, | |
| | | | | | | particularly | |
| | | | | | | those of the | |
| | | | | | | gastrointestinal | |
| | | | | | | and | |
| | | | | | | respiratory | |
| | | | | | | tracts | |
| CTSB | cathepsin B | 37.8 | 1.8 | lysosome & | ✓ | highly | cancer, |
| | [P07858] | | | (1/1) | | expressed | cardiovascular |
| | | | | | | in cervical, | disease, |
| | | | | | | endometrial, | connective |
| | | | | | | liver | tissue |
| | | | | | | melanoma | disorders, |
| | | | | | | and | dermatological |
| | | | | | | pancreatic | diseases, |
| | | | | | | cancer | endocrine |
| | | | | | | | system |
| | | | | | | | disorders, |
| | | | | | | | gastrointestinal |
| | | | | | | | disease, |
| | | | | | | | hematological |
| | | | | | | | disease, |
| | | | | | | | hepatic |
| | | | | | | | system |
| | | | | | | | disease, |
| | | | | | | | infectious |
| | | | | | | | disease, |
| | | | | | | | inflammatory |
| | | | | | | | response, |
| | | | | | | | neurological |
| | | | | | | | disease, renal |
| | | | | | | | and |
| | | | | | | | urological |
| | | | | | | | disease, |
| | | | | | | | respiratory |
| | | | | | | | disease, |
| | | | | | | | skeletal and |
| | | | | | | | muscular |
| | | | | | | | disorders |
| GKN2 | gastrokine-1 | 22.0 | 3 | secreted & | ✓ | slightly up- | gastric |
| | [Q86XP6] | | | (0/1) | | regulated | cancer, |
| | | | | | | in breast | Crohn's |
| | | | | | | cancer and | disease |
| | | | | | | slightly | |
| | | | | | | down- | |
| | | | | | | regulated | |
| | | | | | | in lung | |
| | | | | | | cancer | |
| GHRL | appetite- | 12.9 | 9 | secreted & | ✓ | moderately | antigen |
| | regulating | | | (0/1) | | expressed | presentation, |
| | hormone | | | | | in | cancer, |
| | (Ghrelin) | | | | | colorectal, | cardiovascular |
| | [Q9UBU3] | | | | | liver and | disease, |
| | | | | | | pancreatic | endocrine |
| | | | | | | cancer | system |
| | | | | | | | disorders, |
| | | | | | | | hepatic |
| | | | | | | | system |
| | | | | | | | disease, |
| | | | | | | | inflammatory |
| | | | | | | | disease, |
| | | | | | | | inflammatory |
| | | | | | | | response, |
| | | | | | | | neurological |
| | | | | | | | disease, |
| | | | | | | | nutritional |
| | | | | | | | disease, |
| | | | | | | | organismal |
| | | | | | | | injury and |
| | | | | | | | abnormalities, |
| | | | | | | | psychological |
| | | | | | | | disorders, |
| | | | | | | | reproductive |
| | | | | | | | system |
| | | | | | | | disease, |
| | | | | | | | skeletal and |
| | | | | | | | muscular |
| | | | | | | | disorders |
| LIPF | gastric | 45.2 | 5 | secreted & | ✓ | slightly up- | cardiovascular |
| | triacylglycerol | | | (0/1) | | regulated | disease, |
| | lipase | | | | | in ovarian | endocrine |
| | (Gastric | | | | | caner and | system |
| | lipase) | | | | | down- | disorders, |
| | [P07098] | | | | | regulated | metabolic |
| | | | | | | in breast | disease, |
| | | | | | | cancer | nutritional |
| | | | | | | | disease, |
| | | | | | | | respiratory |
| | | | | | | | disease |
| LIPG | endothelial | 56.8 | 3 | secreted & | ✓ | slightly up- | antigen |
| | lipase | | | (1/1) | | regulated | presentation, |
| | [Q9Y5X9] | | | | | in brain, | cardiovascular |
| | | | | | | ovarian, | disease, |
| | | | | | | and head- | inflammatory |
| | | | | | | neck | response |
| | | | | | | cancer; | |
| | | | | | | slightly | |
| | | | | | | down- | |
| | | | | | | regulated | |
| | | | | | | in leukemia | |
| LIMK1 | LIM | 72.6 | 1.8 | cytoplasm & | ✓ | moderately | cancer, |
| | domain | | | (0/1) | | up- | cardiovascular |
| | kinase 1 | | | | | regulated | disease, |
| | [P53667] | | | | | in | dermatological |
| | | | | | | lymphoma | diseases, |
| | | | | | | cancer and | developmental |
| | | | | | | Melanoma | disorder, |
| | | | | | | | endocrine |
| | | | | | | | system |
| | | | | | | | disorders, |
| | | | | | | | genetic |
| | | | | | | | disorder, |
| | | | | | | | hematological |
| | | | | | | | disease, |
| | | | | | | | neurological |
| | | | | | | | disease, |
| | | | | | | | reproductive |
| | | | | | | | system |
| | | | | | | | disease |
| GAST | gastrin | 11.4 | 1.1 | secreted & | | expressed | cancer, |
| | [P01350] | | | (0/1) | | in stomach | Crohn's |
| | | | | | | cancer | disease, |
| | | | | | | | Zollinger- |
| | | | | | | | Ellison |
| | | | | | | | syndrome |
| TIP47 | mannose-6- | 47.0 | 1.3 | cytoplasm, | | breast, | cervical |
| (M6PRBP1) | phosphate | | | endosome | | cervical, | dysplasia, |
| | receptor- | | | membrane & | | colorectal, | cancer |
| | binding | | | (1/1) | | endometrial, | |
| | protein 1 | | | | | pancreatic | |
| | [O60664] | | | | | malignant, | |
| | | | | | | rental, | |
| | | | | | | testis, | |
| | | | | | | stomach | |
| | | | | | | cancer and | |
| | | | | | | malignant | |
| | | | | | | glioma | |
| PDGFRB | beta-type | 124.0 | 2 | membrane & | ✓ | malignant | cancer, |
| | platelet- | | | (1/1) | | glioma, | cardiovascular |
| | derived | | | | | moderate | disease, |
| | growth | | | | | in ovarian | dermatological |
| | factor | | | | | cancer | diseases, |
| | receptor | | | | | | endocrine |
| | [P09619] | | | | | | system |
| | | | | | | | disorders, |
| | | | | | | | gastrointestinal |
| | | | | | | | disease, |
| | | | | | | | hematological |
| | | | | | | | disease, |
| | | | | | | | hepatic |
| | | | | | | | system |
| | | | | | | | disease, |
| | | | | | | | immunological |
| | | | | | | | disease, |
| | | | | | | | inflammatory |
| | | | | | | | disease, |
| | | | | | | | neurological |
| | | | | | | | disease, |
| | | | | | | | ophthalmic |
| | | | | | | | disease, renal |
| | | | | | | | and |
| | | | | | | | urological |
| | | | | | | | disease, |
| | | | | | | | reproductive |
| | | | | | | | system |
| | | | | | | | disease, |
| | | | | | | | respiratory |
| | | | | | | | disease, |
| | | | | | | | skeletal and |
| | | | | | | | muscular |
| | | | | | | | disorders |
| GIF | gastric | 45.4 | 12 | secreted & | ✓ | down- | genetic |
| | intrinsic | | | (0/1) | | regulated | disorder, |
| | factor[P27352] | | | | | in most of | hematological |
| | | | | | | cancer | disease, |
| | | | | | | tissues, but | metabolic |
| | | | | | | moderately | disease |
| | | | | | | unregulated | |
| | | | | | | in | |
| | | | | | | Leiomyosarcoma | |
| AZGP1 | zinc-alpha- | 33.9 | 3 | secreted & | ✓ | highly | inflammatory |
| | 2- | | | (1/1) | | expression | disease, |
| | glycoprotein | | | | | in prostate | respiratory |
| | [P25311] | | | | | caner and | disease |
| | | | | | | breast | |
| | | | | | | cancer | |

(FC: fold change; annotation* is based on IPA annotation; AS: alternative splicing variants detected. Cancer expression information is retrieved from the Oncomine website and the Proteinatlas website).

### Example 13

### Experimental Validation of Predicted Serum Markers

A combined approach of mass spectrometry and western blot analysis was used to validate the predicted serum protein markers. The serum samples were processed to remove the 12 most abundant proteins (albumin, IgG, α1-antitrypsin, IgA, IgM, transferrin, haptoglobin, α1-acid glycoprotein, α2-macroglobulin, HDL (apoliproteins A-1 & A-II) and fibrinogen) with an antibody column (ProteomeLab™ IgY-12 High Capacity Proteome Partitioning Kit from Beckman Coulter). Specific removal of these 12 highly abundant proteins reduces 96% of total protein mass from human serum or plasma. The predicted biomarkers are present in the remaining 4% of the total protein mass, and thus are easier to identify as a result of the separation step.

After immunocapture of the 12 most abundant serum proteins, the non-specifically bound proteins are eluted from the column and collected. The specifically-bound proteins can also be eluted from the column for further analysis to see if they serve as carriers for the potential biomarkers.

For western analysis, protein samples were incubated at 100° C. for 5 min, separated by SDS-PAGE through 4 to 20% gradient polyacrylamide gels (Bio-Rad), and then transferred onto PVDF membranes. After blocking non-specific binding sites with 3% non-fat dry milk in TBST (10 mM Tris HCl, pH 7.5, 150 mM NaCl, 0.05% Polyoxyethylene sorbitane monolaurate (Tween-20) [wt/vol]) for 2 hour at room temperature, membranes were incubated overnight at 4° C. with primary antibodies (diluted 1:200, 1:500, 1:3000, 1:10000, varying in each antibody) in 1.5% non-fat dry milk in TBST. After three washes with TBST, the membranes were incubated in 1.5% non-fat dry milk in TBST containing secondary antibodies for 2 hours at room temperature. The membranes were then subjected to an enhanced chemiluminescence reaction using western Lightning Chemiluminescence Reagent Plus (Perkin Elmer, USA). The MagicMark western protein standard (Invitrogen, Karlsruhe, Germany) was used to identify the molecular weights. The ECL membrane images were evaluated for the quantification of protein concentration using the Gel Analysis function of the ImageJ 1.34s software (available on the NIH website). The antibodies were from Abnova, Inc. (Taipei, Taiwan), Santa Cruz Biotechnology, Inc. (Santa Cruz, Calif.) and Abcam, Inc. (Cambridge, Mass.). The predicted splice variants were used in the antibody selection. If the most abundant splicing isoforms are too short to cover any antigenic region (epitopes), the marker might not be detected through antibodies specifically designed for the full-length protein. Thus, those antibodies were chosen whose epitope regions are covered by the majority of the transcripts based on analyses of the predicted splice variants.

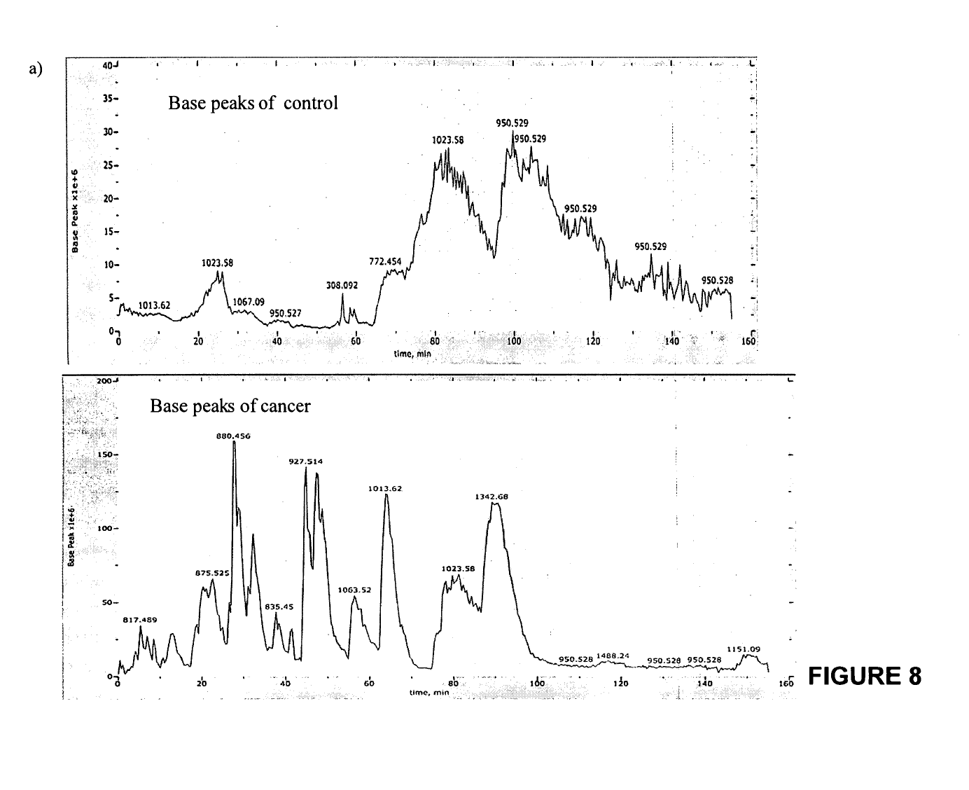

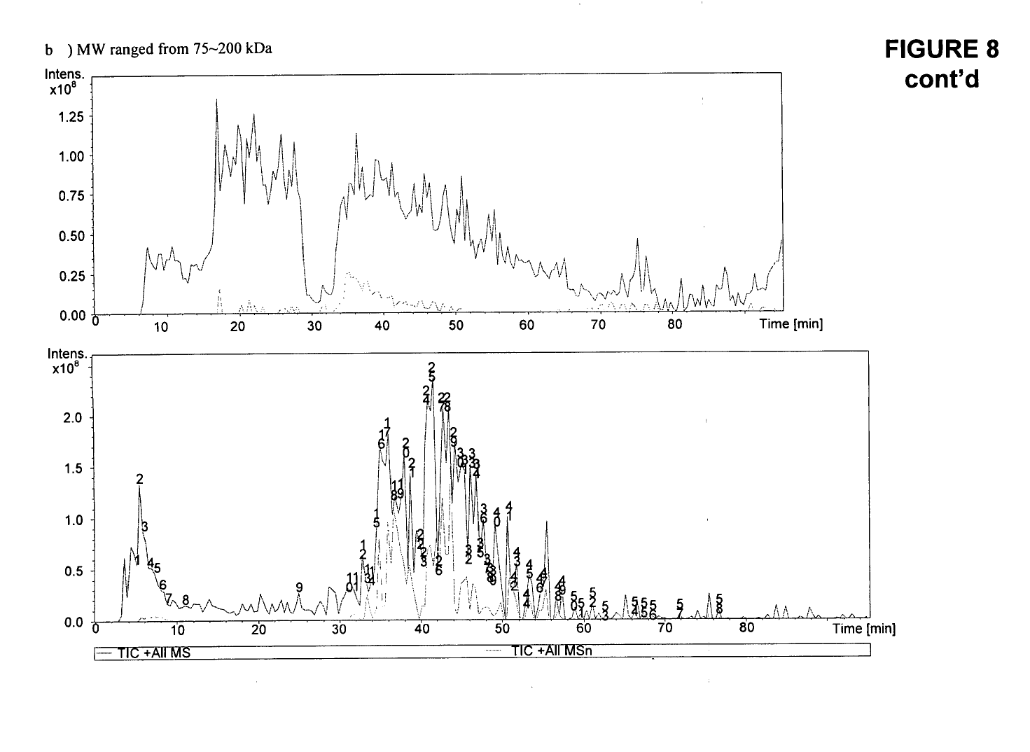

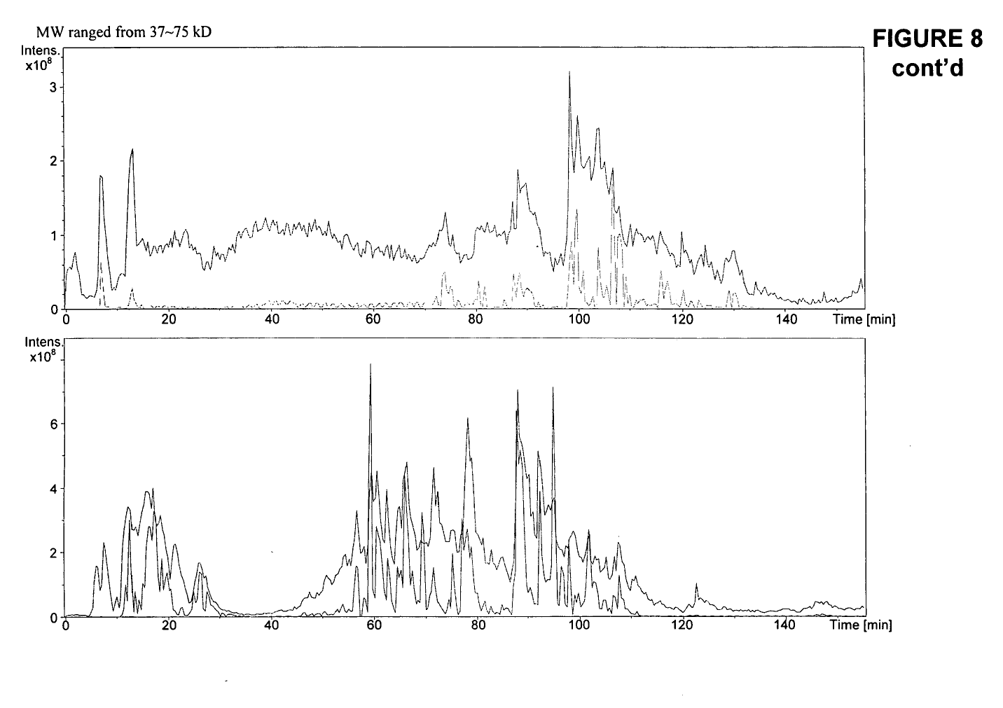

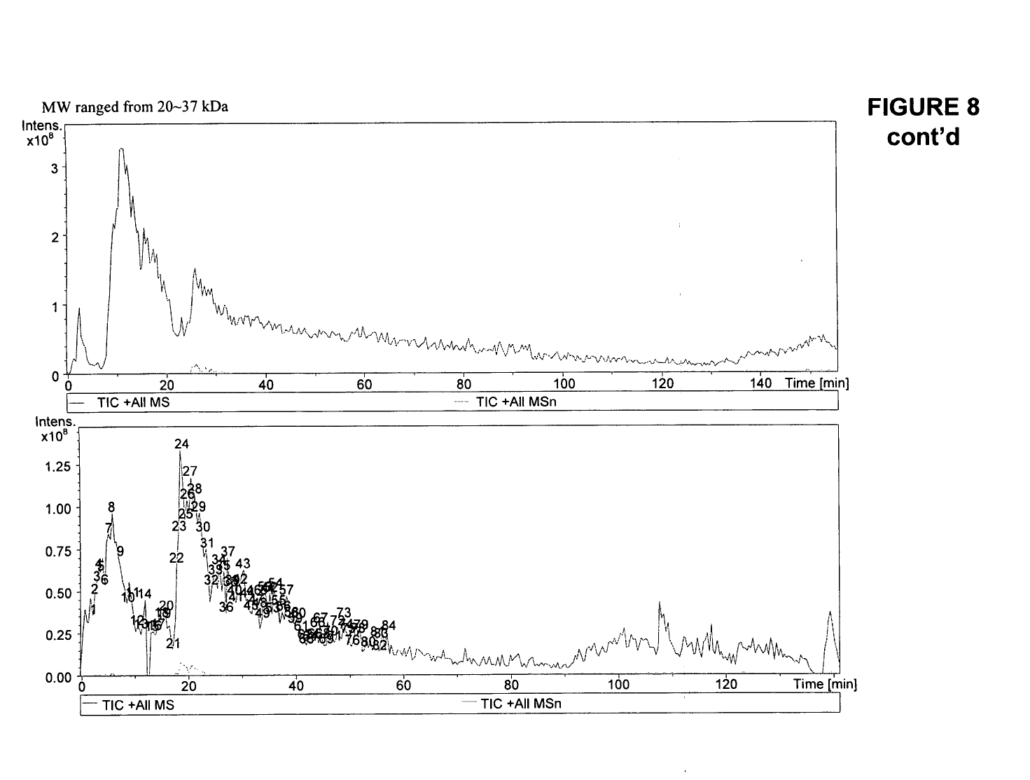

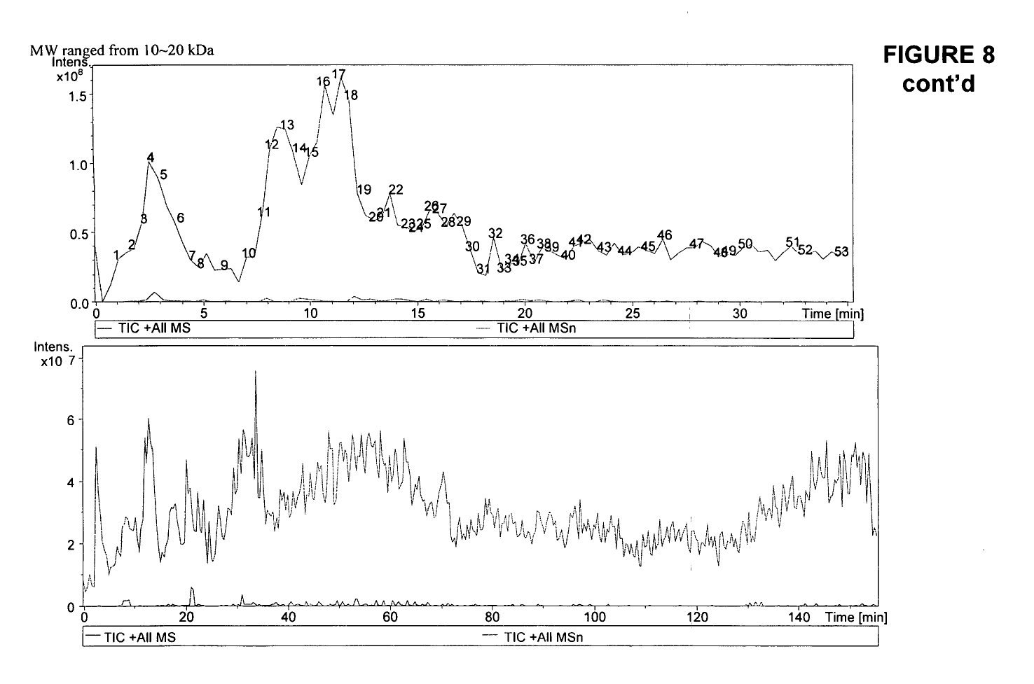

MS experiments were conducted on the proteins extracted from the gel by two different approaches. After digestion with sequencing grade, modified trypsin, protein samples were subjected to online HPLC analysis using an Agilent 1100 series HPLC with a 75 um C-18 reverse phase column directly coupled to a 9.4 T Bruker Apex IV QeFTMS (Billerica, Mass.) fitted with an Apollo II nanoelectrospray source. Collisionally activated dissociation (CAD) was used for ion dissociation, and protein fragmentation was done using argon as a collision gas, followed by their injection into the ICR analyzer cell. Data analysis was accomplished using Bruker Data Analysis Software and the MS-Tag program on the Protein Prospector Website for protein identification. In parallel, the same samples were digested with proteomics-grade Trypsin (Promega) and analyzed on an Agilent 1100 capillary LC (Pal Alto, Calif.) interfaced directly to a LTQ linear ion trap mass spectrometer (Thermo Electron, San Jose, Calif.). The peptide samples were loaded using positive N2 pressure on a PicoFrit 8-cm by 50-μm column (New Objective, Woburn, Mass.) packed with 5-μm diameter C18 beads. Peptides were eluted from the column into the mass spectrometer during a 55 min linear gradient from 5% to 60% of total solution composed of mobile phase B at a flow rate of 200 mL min-1. The instrument was set to acquire MS/MS spectra on the nine most abundant precursor ions from each MS scan with a repeat count of 3 and repeat duration of 15 s. Dynamic exclusion was enabled for 20 s, and data analysis was conducted by Mascot (see the website of matrixscience) (FIG. 8).

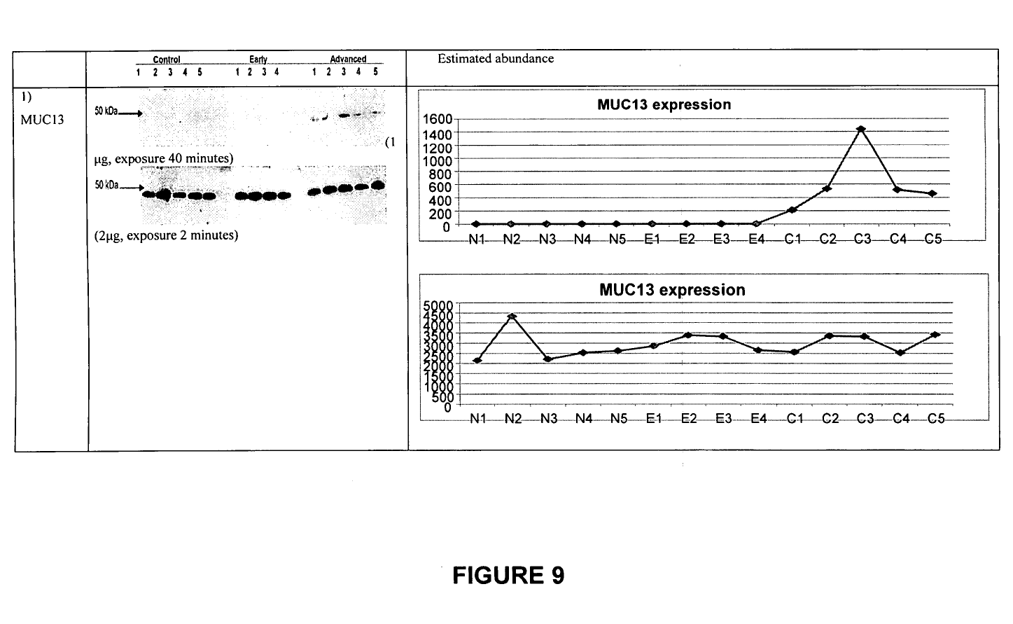

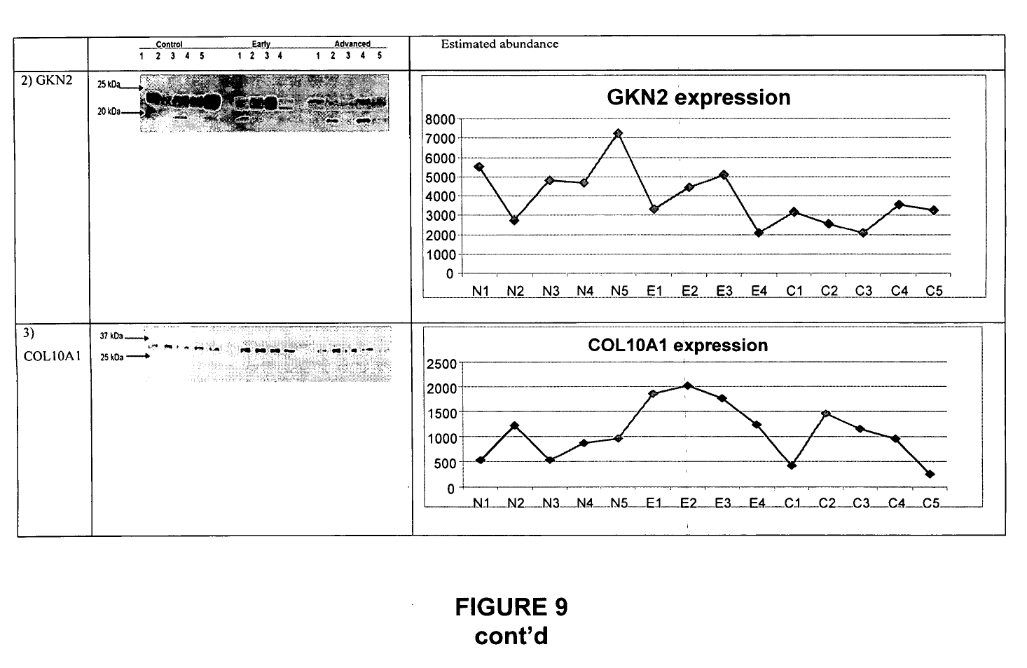

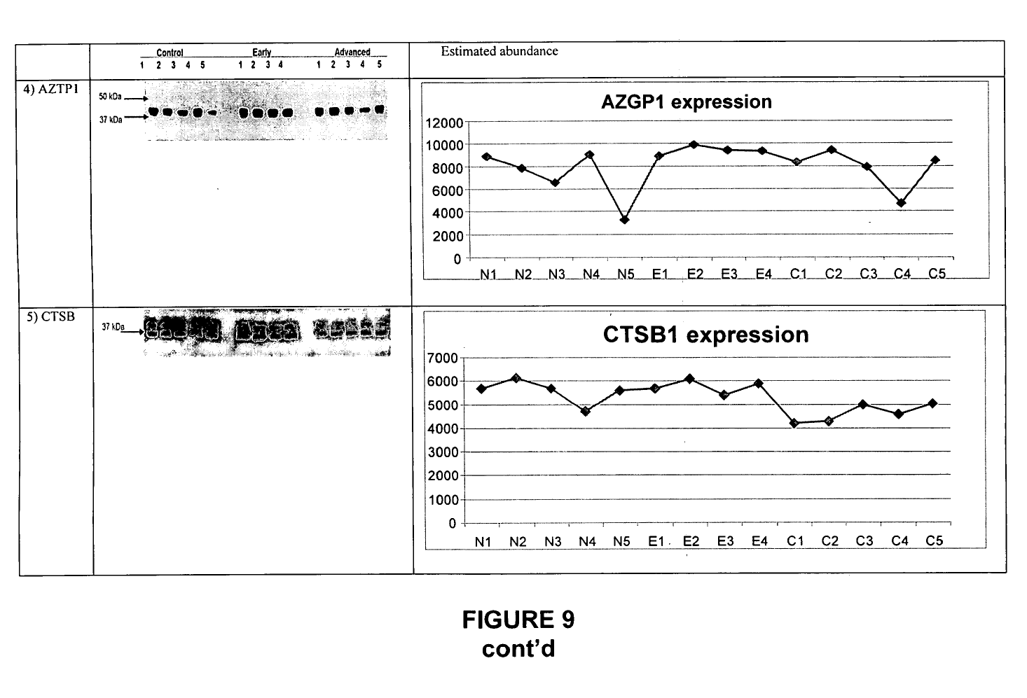

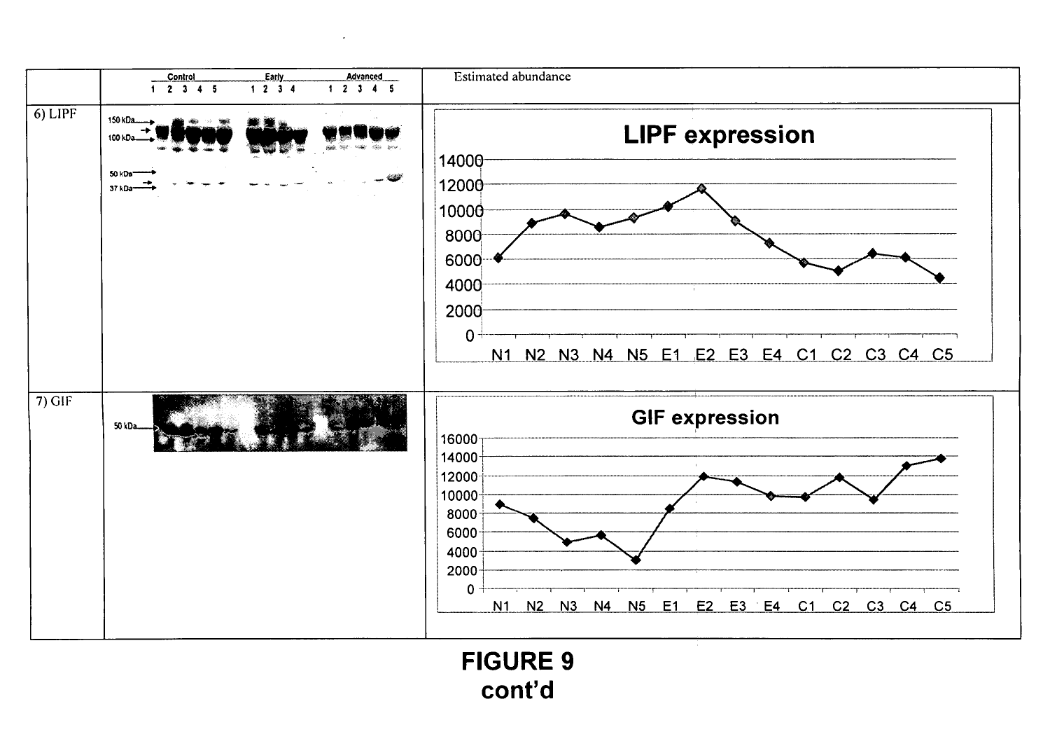

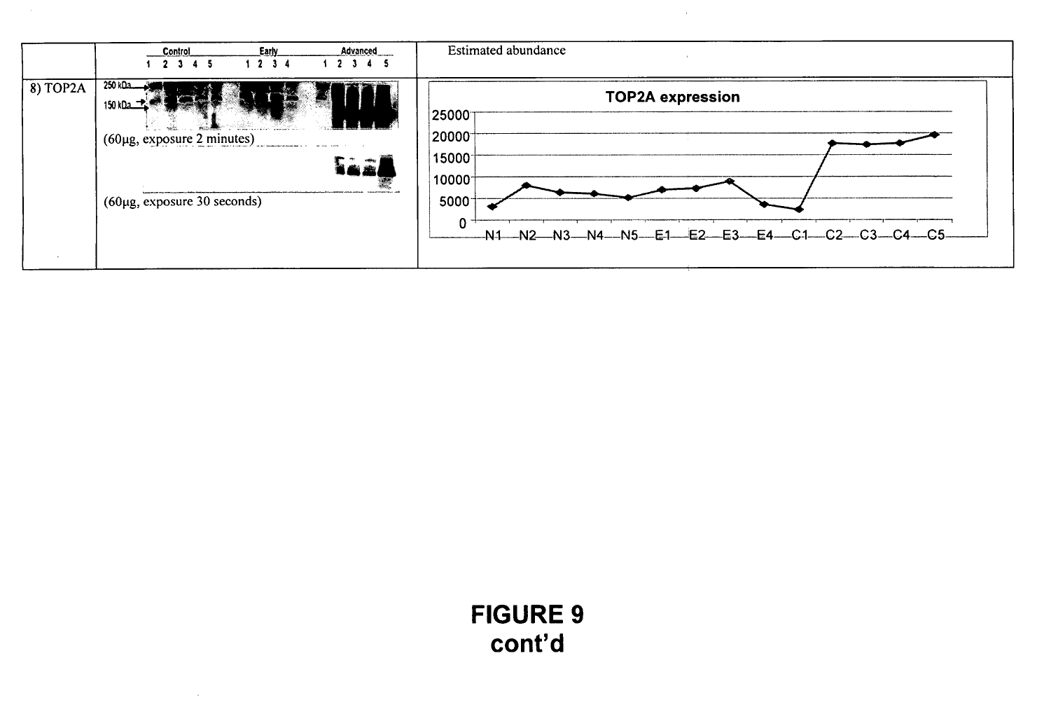

The validation set consists of serum samples from nine gastric cancer patients (4 early and 5 advanced cancers) and five age- and gender-matched controls. This validation set includes a few additional samples to those pooled for mass spectrometry analyses, as an independent evaluation set. The 20 most promising candidate markers were selected for western blot analysis based on our computational prediction, four of which were detected by the above MS analyses. 15 of these proteins are found in the serum samples, including two detected by MS-based analysis (TOP2A and AZGP1). Among them, seven (GKN2, MUC13, LIPF, GIF, AZGP1, CTSB, and COL10A1) show some level of differential abundance between the sera of the cancer patients and the control sample as shown in FIG. 9.

As can be seen in FIG. 9, there are two types of potential markers: (1) proteins with increased/decreased abundance in advanced cancer. For instance, Mucin-13, showing increased abundance in the advanced cancer sera, is a glycoprotein that covers the apical surface of the trachea and gastrointestinal tract, playing roles in several signaling pathways that affect oncogenesis, motility, and cell morphology. It could be used as a general cancer marker but may not be effective for early stage cancer detection. Gastric lipase (LIPF) and DNA topoisomerase 2-alpha (TOP2A) are also differentially expressed in advanced stage cancer sera, with decreased and increased expression, respectively. (2) proteins with differential expression in early stage cancer, namely GKN2, COL10A1 and AZTP1. GKN2, with decreased expression in caner sera, could be effective for detection of early-stage cancer since the abundance changes in half of early stage samples in our test, including one stage-I cancer.

Among these promising markers, CTSB has been proposed as a potential gastric cancer marker (Ebert et al., 2005; Poon et al., 2006), which shows differential abundance but not consistent across our samples; MMP1 and TOP2A have been previously proposed as cancer related in general (Poola et al., 2005); the data presented herein support this. GKN2 and LIPF are gastric tissue specific; and COL10A1 and GAST may be associated with other diseases or immune response in general.

Combinations of these individual proteins have been considered as potential combinatorial markers. While detailed quantitative assessment of combinatorial markers are challenging due to the lack of accurate quantity measurements of these proteins, the classification accuracies have been roughly evaluated based on the estimated protein abundance from the western blot data. As shown in Table 4, a set of k-protein markers are listed, which give much improved classification accuracies than individual serum markers. Table 10 gives the detailed list of the k-protein serum markers.

TABLE 10
Detection accuracies of the validated k-protein markers, which are
evaluated at both the gene- and the protein-level, based on 5-cross
validation accuracy.

| | | Detection accuracies | |
|---|---|---|---|
| k | Markers | Proteins-level | Gene-level |
| 1 | GIF | 0.867 | 0.726 |
| | GKN2 | 0.80 | 0.705 |
| | MUC13 | 0.667 | 0.613 |
| 2 | GIF + LIPF | 0.933 | 0.746 |
| | GIF + COL10A1 | 0.867 | 0.732 |
| | GIF + TOP2A | 0.80 | 0.732 |
| 3 | GIF + LIPF + MUC13 | 0.933 | 0.733 |
| | LIPF + GIF + AZGP1 | 0.867 | 0.719 |
| | COL10A1 + GKN2 + GIF | 0.80 | 0.753 |
| 4 | LIPF + GIF + MUC13 + AZGP1 | 0.933 | 0.767 |
| | LIPF + GIF + MUC13 + COL10A1 | 0.933 | 0.788 |
| | LIPF + GIF + MUC13 + GKN2 | 0.80 | 0.740 |

It should be noted that some factors may affect the western blot results. For example, one such factor is that different splicing isoforms may not necessarily have similar binding affinity to the antibodies designed for the full-length common form of each related protein. Markers such as MMP1, LIPG, LIPF, and CTSB all have splicing variants based on the presented predictions. Thus, appropriate antibodies were chosen based on the predicted splicing variants.

### Example 14

### Identification of Cancer Markers in Urine

Collection of training and testing data. A set of 1,500 proteins that were identified from a major urine proteomics study (Adachi et al. 2006) were used as the positive training data. A total of 1,313 human proteins were identified in this proteomics study with SwissProt accession IDs and were included in the training set. For an independent test set, data from three other major urinary proteomics studies (Pieper et al., 2004; Castagna et al., 2005; Wang et al., 2006) were used, including a total of 460 human proteins that do not overlap the training set.

For negative training and test datasets, proteins were collected from Pfam families that do not overlap the positive data following a selection procedure described in Cui et al., 2008, to ensure that the selected proteins follow the same family-size distribution in the Pfam (Finn et al., 2008). As a result, 2,627 and 2,148 proteins were selected for the training and the testing set, respectively, without any overlap between the two sets.

Feature calculation and selection. For each protein sequence retrieved from the SwissProt database, 18 features were calculated. Some of these features need multiple feature values to represent them, e.g., 20 feature values to represent the amino acid composition in a protein sequence; hence the 18 features are represented using 243 feature values. Table 11 lists the 18 features and the number of feature values used to represent each of them. The 18 features were calculated using either in-house programs or prediction servers if available on the Internet.

This list of features is potentially useful in distinguishing between urine-excreted proteins and the non-urine-excreted proteins, selected based on the information available about urine excretion. To check which of them are actually useful, the feature selection tool provided in a Library for Support Vector Machines (LIBSVM) to select the useful features among the 243 feature values were used. LIBSVM is an integrated software for support vector classification (C-SVC, nu-SVC), regression (epsilon-SVR, nu-SVR), and distribution estimation (one-class SVM). The feature-selection tool calculates an F-score (Chang & Lin 2001) to measure the ranking of the relevance of each feature value to our classification problem. All the features with F-scores lower than a pre-selected threshold were removed, and the remaining features were considered as useful for the classification problem.

TABLE 11
Summary of features used in the initial classification model.

| | Feature names and | Program used to calculate |
|---|---|---|
| Feature class | feature values | the features |
| Sequence | Sequence Length (1) | Fldbin (Prilusky et al. |
| features | AA composition (20) | 2005), Profeat (Li et |
| | | al., 2006) |
| Physicochemical | Hydrophobicity (21), | Locally calculated, Profeat |
| properties | normalized Van der | (Li et al., 2006): using three |
| | Waals volume (21), | descriptors: composition, |
| | polarity (21), | transition, and distribution |
| | polarizability (21), | |
| | charge (21), | |
| | secondary structure | |
| | (21), solvent accessibility | |
| | (21), Pseudo-AA | |
| | descriptor (50) | |
| | Unfoldability (1), charge | Fldbin (Prilusky et al., |
| | (1), hydrophobicity (1), # | 2005), Swiss (Gasteiger et |
| | of disordered regions (1), | al., 2003), locally |
| | longest disordered | calculated |
| | regions (1), # of | |
| | disordered residues (1), | |
| | PI (1), MW (1), charge | |
| | (2), percentage of | |
| | disordered region (1) | |
| Motifs | Transmembrane domain | TMB-Hunt (Bendtsen et al., |
| | (1), Twin-arginine signal | 2005; Garrow et al. 2005), |
| | peptide (1), | TatP (Bendtsen et al., |
| | transmembrane domains | 2005), phobius (Kall et |
| | (alpha helix, or beta | al., 2007), NetOgly |
| | barrel) (2), Glycosylation | (Julenius et al., 2005), |
| | number & presence | NetNGly (Gupta et al., |
| | (N&O linked) (4) | 2004) |
| Structural | Secondary structural | SSCP (Eisenhaber et al., |
| Option 2. | content (4), Radius | 1995), Radius Gyration, |
| 243 | gyration (1), Radius (1) | locally calculated |

The DAVID Bioinformatics Resources web server was used to do functional enrichment analysis for all the predicted urine-excreted proteins. The functional annotation clustering analysis was performed using the human proteins as the background. The overall enrichment score for the group was determined by the EASE scores for each cluster (Dennis et al., 2003; Huang et al., 2009).

The KOBAS web server (Mao et al., 2005; Wu et al., 2006) was used to find statistically enriched and underrepresented pathways among the predicted urine-excreted proteins. KOBAS takes in a set of sequences and annotates KEGG orthology terms based on BLAST sequence similarity. The annotated KO terms were then compared against all human proteins. A pathway is considered enriched or underrepresented if there is at least a 2-fold change in terms of the percentage composition.

Urine samples from 10 gastric cancer patients (7 male, 3 female) in metastasis stage and 10 gender-matched healthy people were collected at the Medical School of Jilin University, Changchun, China. These samples were immediately lyophilized and stored until they were ready to use. The samples were reconstituted and were spun at 3,000 relative centrifugal forces for 25 minutes at 4° C. to remove cellular components. The supernatants were collected and frozen at −80° C. until further use. The samples were then dialyzed at 4° C. against Millipore ultra pure water (three buffer changes followed by an overnight dialysis) using Slide-A-Lyzer Dialysis Cassettes (Thermo Fisher Scientific, Rockford, Ill.). Protein concentrations were measured using the Bio-Rad Protein Assay (Bio-Rad, Hercules, Calif.) with bovine serum albumin as a standard.

Signal Peptide and secondary structures are key features of urine-excreted proteins. Using the F-score-based feature selection, the highest accuracy was observed when the number of feature values was 74. Using these 74 feature values, the SVM-based classifiers were retrained. Among the selected features, the most discriminatory for the excreted proteins was the presence of the signal peptide. It is known that proteins that are secreted through the ER have signal peptides and are trafficked to their destination according to the specific signal peptide; thus, most excreted proteins will have this feature. Another prominent feature was the type(s) of secondary structure; several feature values associated to the secondary structure were included among the top 74, and the percentage of alpha helices was ranked at number 2 among the 74.

The charge of a protein was among the top ranked features for excreted proteins. This is consistent with the general understanding that charge is indeed a factor in determining which proteins are filtered through the glomerulus membrane in the kidney. However, the molecular size of proteins, ranked at 232, and was found as irrelevant to the classification problem.

As shown in Table 12, two classifiers were trained. Model 1 has higher specificity but lower sensitivity, whereas model 2 shows more balanced performance. Due to the unbalanced numbers of the positive and the negative training data, the accuracy may not be the best measure to determine the performance of a model. Thus, Matthew's correlation coefficient is used as a measurement of classification quality.

TABLE 12
The performance of the trained models on the training.

| Sets | Model | TP | TN | FP | FN | SEN | SP | ACC | MCC |
|---|---|---|---|---|---|---|---|---|---|
| Train | 1 | 792 | 2493 | 134 | 341 | 0.7403 | 0.9490 | 0.8794 | 0.5228 |
| Train | 2 | 1164 | 2230 | 297 | 149 | 0.8865 | 0.8869 | 0.8868 | 0.5697 |
| Independent | 1 | 360 | 1983 | 165 | 100 | 0.7826 | 0.9232 | 0.8984 | 0.4500 |
| Independent | 2 | 404 | 1838 | 310 | 56 | 0.87820 | 0.85567 | 0.85966 | 0.39358 |

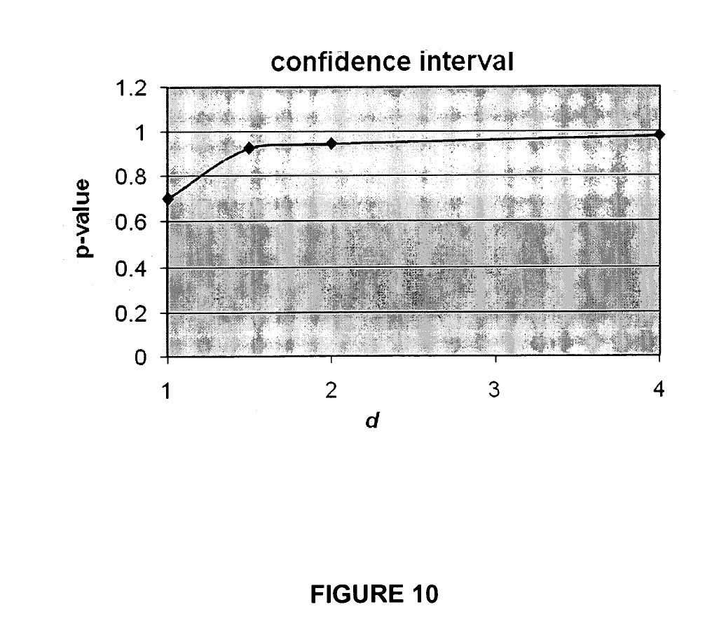

There is a direct correlation between the confidence of a prediction and the distance of the protein from the separating hyperplane between the positive and the negative training data as derived by the SVM-based training. Specifically, the further the distance is from the separating hyperplane, the higher the probability of a correct prediction (FIG. 10). Using the confidence interval as a guide, a few proteins can be selected for experimental validation.

Application of trained classification models to stomach cancer data. In an effort to identify potential biomarkers for stomach cancer in urine, the trained models developed herein were applied to a set of 2,048 differentially expressed genes identified based on 160 exon arrays on 80 stomach cancer tissues and 80 matching noncancerous stomach tissues from the same 80 patients on an Affymetrix Human exon array 1.0 (Cui et al., 2009). Among the 2,048 proteins, 480 were predicted to be excreted into urine by Model 1; of these 480 proteins, 11 proteins have a confidence level above 98%, suggesting that they are highly likely to be excreted into urine. A total of 203 proteins out of the 480 have a confidence level at least 92%, which is also considered as a highly reliable prediction.

Functional and pathway enrichment analyses were performed on all the 480 proteins to aid in determining which types of proteins could be found in urine. Specifically, if the analysis suggests that a specific functional group or a pathway is enriched, the chances for finding a biomarker in that group will increase. The functional and pathway enrichment analyses were analyzed using DAVID (Dennis et al., 2003) and KOBAS (Wu et al., 2006) web servers, respectively, using the intact human protein as the background.

The functional enrichment analysis by DAVID revealed that the most enriched functional groups among the 480 proteins were involved with the extracellular matrix (ECM). The ECM plays an important role in cancer progression by affecting cell proliferation and motility. The interaction between the cell surface receptors with ligands in the ECM not only affects cell detachment and migration, but the ECM also serves as a template on which cells can attach and grow (Ashkenas et al., 1996; McKinnell et al., 2006). The composition of the ECM molecules, cell type, and cell-surface receptor composition can promote or inhibit cell proliferation by sending signals through integrins (Stein & Pardee 2004). Thus, proteins involved with the ECM may be an important urine biomarker not only for stomach cancer, but for all other types of cancers as well. Overall, 164 of the 480 proteins are in this group.

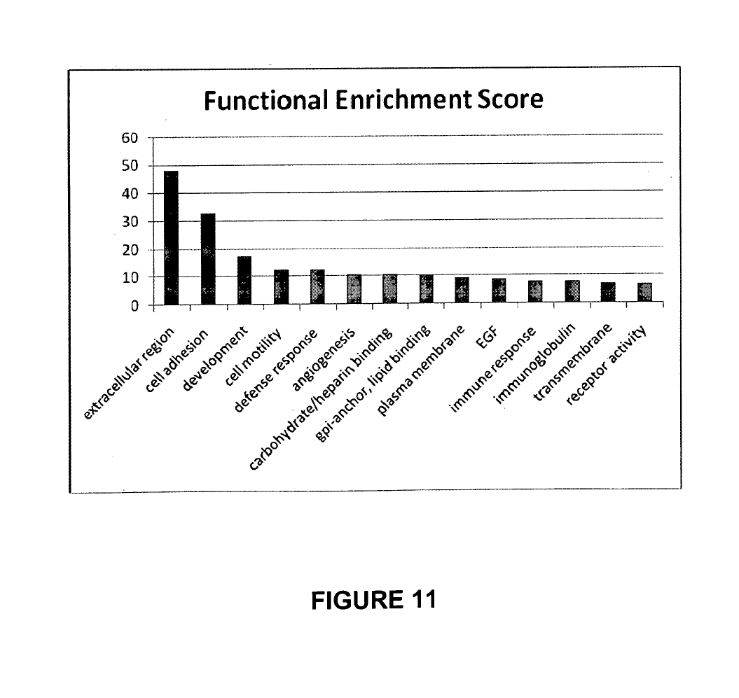

The next most enriched group was proteins involved in cell adhesion. The cell adhesion proteins are well known to be a factor contributing to the cancer growth. For example, cells adhere to each other and to the ECM, but when tumors form, the cells must disassociate from the primary tumor and invade the lymph system in order to metastasize. Consequently, carcinoma cells do not express cell adhesion molecules, such as E-cadherin, and lose their characteristic morphology and become invasive (Frixen et al., 1991). Among the 480 proteins identified, 93 are in this group, thus providing cautious optimism of finding a cell adhesion biomarker in urine Other enriched functional groups include proteins involved in development, cell motility, defense/inflammatory response, and blood vessel development/angiogenesis. FIG. 11 shows the overall results of the functional enrichment analysis.

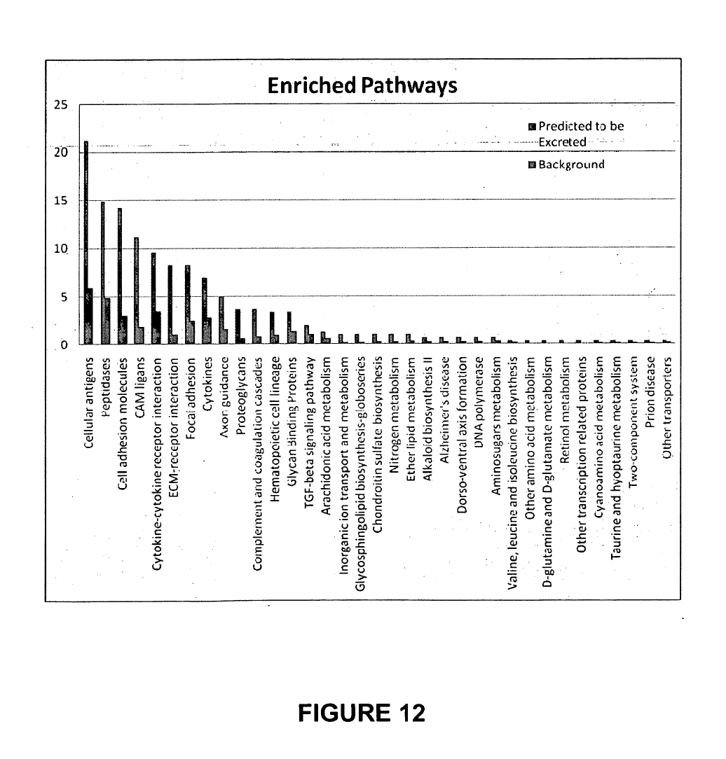

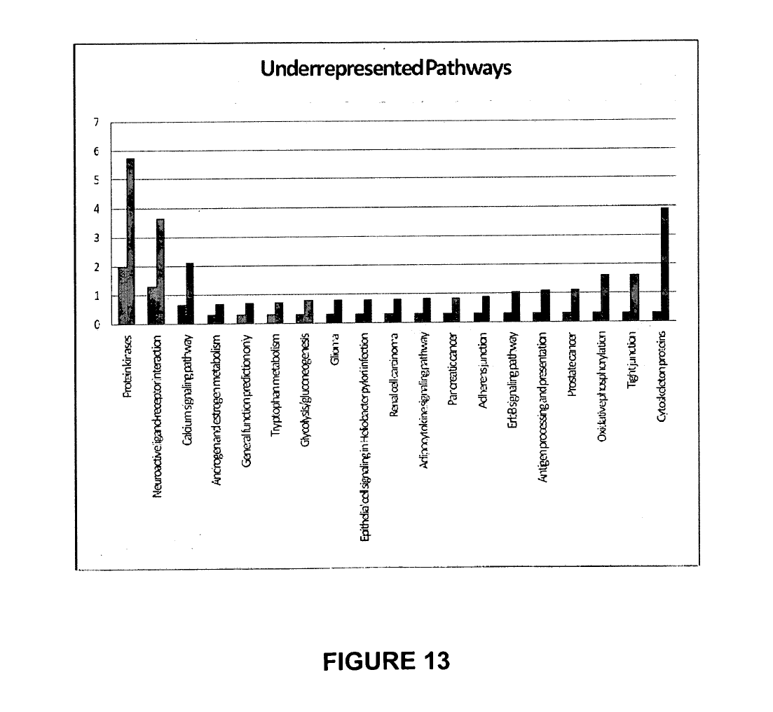

The pathway enrichment analysis of the 480 proteins reveals that certain pathways are statistically enriched (FIG. 12) or underrepresented (FIG. 13) compared to the background, the whole human protein set. Among the 480 proteins, more than 20% were involved in the cellular antigens pathway, which may be triggered by the immune system in response to cancer formation and development. The role of the immune system in cancer development is not well understood, particularly since it can have paradoxical roles on cancer development and progression. For example, the activation of anti-tumor adaptive immune responses can suppress tumor growth and development, and, while the abundance of infiltrating lymphocytes correlates with more favorable prognosis, an increased abundance of infiltrating innate immune cells correlates with increased angiogenesis and poor prognosis (de Visser et al., 2006).

The enrichment of proteins in the antigen pathway is not surprising due to their easy access to the bloodstream. While in blood circulation, they could easily be filtered through the glomerulus, unlike the intracellular proteins. This indicates that there are more antigen cancer markers that remain to be discovered. Peptidases, cell adhesion molecules, and CAM ligands are overrepresented in the pathway analysis, as expected due to their role in cancer progression.

Most of the underrepresented proteins are intracellular proteins (FIG. 13). For example, the protein kinase pathway is significantly underrepresented in the 480 proteins. Protein kinases are involved in crucial intracellular processes such as ion transport, cellular proliferation, hormone responses, apoptosis, metabolism, transcription, and cytoskeletal rearrangement and cell movement (Malumbres & Barbacid, 2007). Deregulation of kinase activity often leads to tumor growth. For example, there is evidence that many kinase mutations are the ‘driver’ mutations contributing to the development of cancer (Greenman et al., 2009); moreover, inhibitors of mutated protein kinases have shown efficacy in cancer treatment (Sawyers, 2004). Regardless of its crucial role in cancer progression, an underrepresentation of protein kinase pathways is due to the fact that these proteins are intracellular and thus unlikely to be excreted into urine.

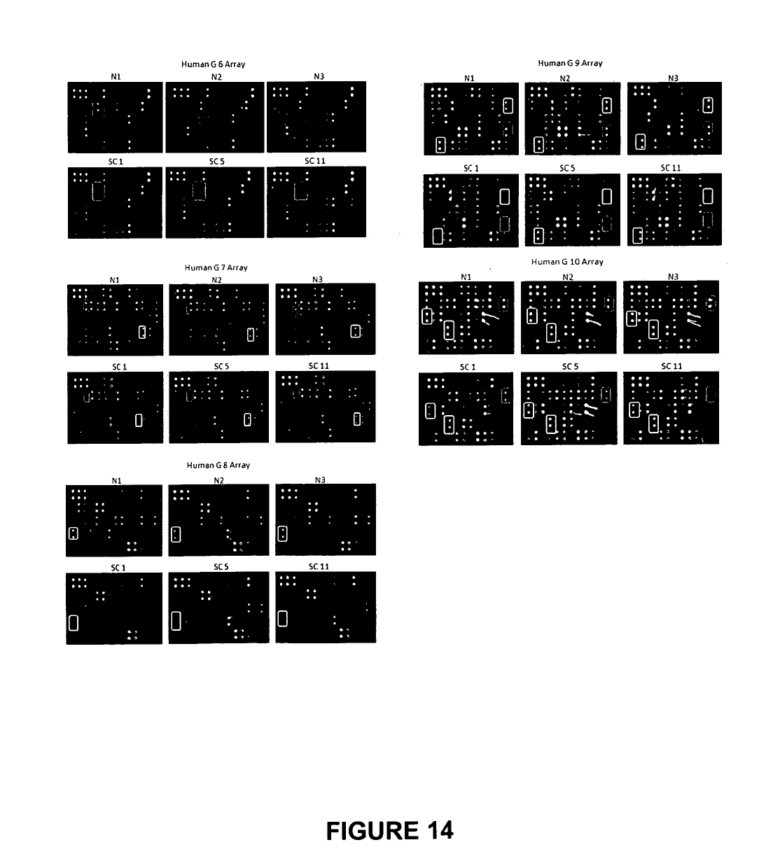

Antibody array screening. Among the 2,048 genes differentially expressed between the gastric cancer tissue and normal tissue, 26 proteins were included in the 274 antibody array (FIG. 14). Of these 26 proteins, seven (FGF7, CD14, MMP9, MMP2, MMP10, TREM1, CEACAM1) were predicted by our model to be excreted. The antibody array data confirmed that 6 of the 7 proteins predicted to be excreted were present in urine in at least one or more samples. However, MMP10 was not detected in any of the six samples, suggesting it to be a false positive. Nevertheless, the model was accurate in predicting excreted urinary proteins.

From the antibody array, 10 proteins (Fit3-ligand, EGF-R, sgp130, PDGF AA, lutenizing hormone, Tim-3, Trappin-2, CEA, CEACAM1, FSH) were found to be substantially down-regulated in all cancer samples, compared to the normal samples (FIG. 14), suggesting these as a possible new biomarkers, but at reduced concentrations, in gastric cancer. Of these 10 proteins, CEACAM1 was the only protein included in the data set of 2,048 differentially expressed genes between the gastric cancer and the reference samples (Cui et al., 2009). This protein was predicted to be excreted by the model implying the success of the model in identifying potential biomarker in urine.

Western blot analyses were performed on a few of the predicted urine-excreted proteins. Three proteins, MUC13, COL10A1, and EL, were selected based on the ranking of the urine-excretion prediction and protein functions. The transmembrane mucin MUC13 has been shown to be up-regulated in stomach cancer tissues and has been suggested as a potential diagnostic and therapeutic target (Shimamura et al., 2005). It has three EGF-like domains that are likely to be involved in cell adhesion, modulation, cell signaling, chemotaxis, wound healing and mucin/growth factor interactions (Williams et al., 2001; N'Dow et al., 2004).

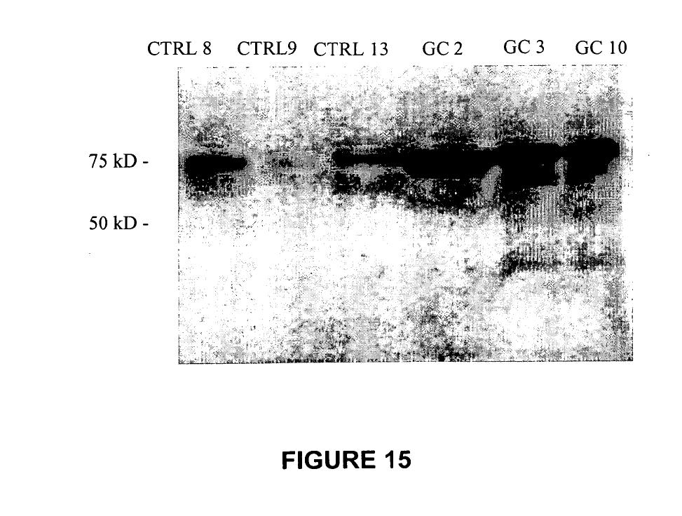

MUC13 (58 kD) was predicted to be excreted into urine, and Western blot confirms the prediction. As shown in FIG. 15, MUC13 is present in urine samples for both stomach cancer patients and the controls. The relative quantification of bands was determined using the ImageJ software, where each lane was analyzed and the area under the peak determined and compared. Although, the microarray data revealed that the MUC13 showed differences in the mRNA level, the quantification of the Western blot bands did not show a significant difference between the cancer samples and the control samples of the band at 58 kD. Since the band is located between the 55-75K, these results suggest that the protein is excreted into urine in an intact, or nearly intact, form.

COL10A1 is a homotrimeric collagen with large C-terminal and N-terminal domains (Gelse et al., 2003). It is thought to be involved in the calcification process in the lower hypertrophic zones and has been found to be localized to presumptive mineralization zones of hyaline cartilage (Schmid & Linsenmayer, 1987; Kwan et al., 1989; Kirsch & Mark, 1992; Alini et al., 1994). It has been found to be over-expressed in breast cancer and ovarian cancer tissues (Ferguson et al., 2005). Our microarray data also shows COL10A1 to be over-expressed in stomach cancer tissues.

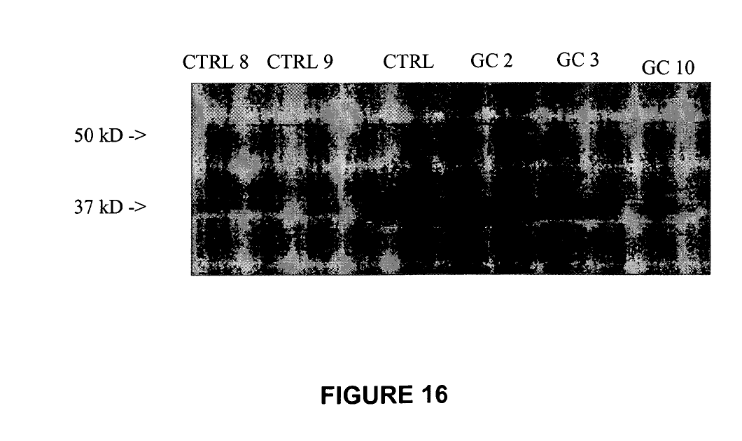

Western blots on COL10A (66 kD) show a clearer band between 37-50 kD, suggesting that this protein is mostly found in urine in an incomplete form probably due to one or more cleavages (FIG. 16). The average intensity of the stomach cancer samples was ˜50% higher when compared to the control samples.

Endothelial lipase (EL) (55 kD) is produced by endothelial cells and functions at the site of their synthesis in general lipid metabolism (Choi et al., 2002; Ishida et al., 2003). Several studies have shown that this protein is a determinant factor in controlling HDL level and there is an inverse relationship between the expression of EL and HDL (Ishida et al., 2003; Jin et al., 2003; Ma et al., 2003). EL has also been associated with macrophages in human atherosclerotic lesions; suppression of EL decreased the expression of pro-inflammatory cytokines in human macrophages and reduced intracellular lipid concentration (Qiu et al., 2007).

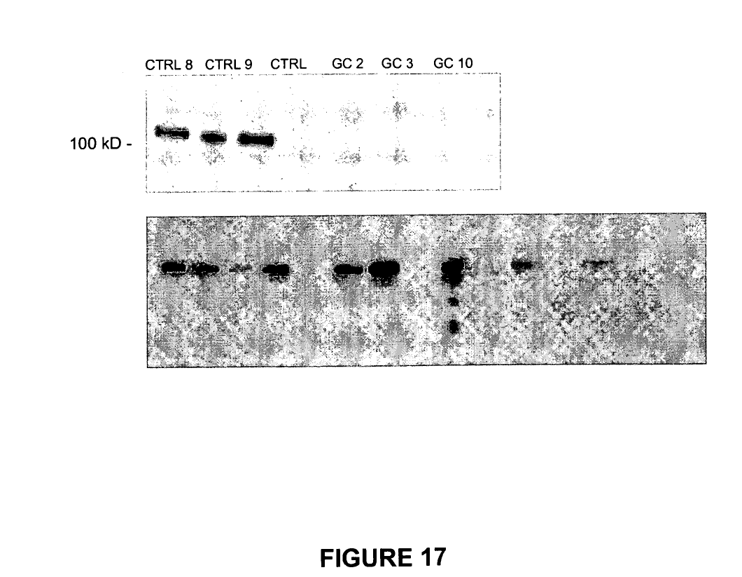

This protein has not been linked to any cancer yet, but this protein was found to be up-regulated in stomach cancer tissues based on our microarray data analysis (Cui et al., 2009). Interestingly, Western blot for EL showed substantial reduction in its abundance in urine samples of stomach cancer patients compared to the control samples (FIG. 17). Specifically, the EL was detected for all three control samples while stomach cancer samples showed little or no EL. Surprisingly, the bands were detected above 100 kD, suggesting that the EL was excreted to urine in an active form, a homodimer in a head-to-tail conformation (Griffon et al., 2009); no other bands were observed for any of the samples.

### Example 15

### Antibody Array Experiments for Marker Identification

Protein array experiments were also carried out using Biotin label-based antibody arrays on the serum samples from three gastric cancer individuals and three controls. For the biotin-labeled-based array experiment, each serum sample was dialyzed, followed by a biotin-labeled step according to the manufacturer's instructions (Pierce, Rockford, Ill., USA), where the primary amine of the proteins is biotinylated. The biotin-labeled proteins (50 μl of serum sample) were then incubated with antibody chips (RayBio® Biotin Label-Based Antibody Arrays, RayBiotech, Inc. U.S.A at room temperature for 2 h. After the incubation with HRP-streptavidin or Fluorescent Dye-Strepavidin, the signals were visualized either by chemiluminescence or fluorescence, and were then imaged by Scan Array laser confocal slide scanner (PerkinElmer Life Science). All the array experiments were repeated three times.

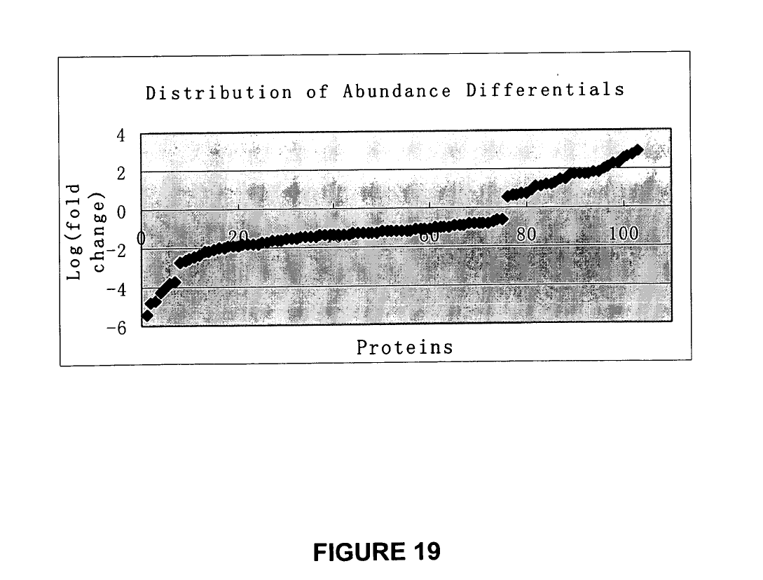

The abundances of 507 known human proteins were measured, including (anti-) inflammatory cytokines, chemokines, adipokines, matrix metalloproteinases, angiogenic factors, growth and differentiation factors, cell adhesion molecules and soluble receptors. The analysis identified 103 proteins with highly significant differences in expression between the gastric cancer and control samples, among which 28 proteins were more abundant in cancer samples while the others showed lower abundance in cancer versus control samples. The distribution of the abundance differentials is shown in FIG. 19, and the list of these protein names is given in Table 13.

Only one of these 103 proteins (CCL28) is detected by our mass spectrometry analysis, which may be due to the relatively lower abundance of the signaling proteins in the samples. Based on this study, it may be concluded that while the antibody array could potentially detect protein markers, its specificity could be a concern.

TABLE 13
103 proteins identified with differential abundances in cancer sera versus
control sera through Biotin label-based antibody array

| | Mean | Mean | Fold |
|---|---|---|---|
| Protein ID | control | cancer | change |
| Insulysin/IDE | 96.7 | 747.3 | 7.7 |
| IL-20 R alpha | 199.0 | 1314.0 | 6.6 |
| IL-31 RA | 41.3 | 263.0 | 6.4 |
| IL-16 | 244.3 | 1404.3 | 5.7 |
| SDF-1/CXCL12 | 1584.3 | 7729.3 | 4.9 |
| SCF | 585.3 | 2782.7 | 4.8 |
| IL-17RC | 29.0 | 120.0 | 4.1 |
| TECK/CCL25 | 49.0 | 195.0 | 4.0 |
| RELT/TNFRSF19L | 73.7 | 262.0 | 3.6 |
| IL-18 BPa | 1622.3 | 5707.0 | 3.5 |
| TGF-alpha | 54.7 | 185.3 | 3.4 |
| FGF-12 | 101.7 | 344.3 | 3.4 |
| IL-17RD | 1039.0 | 3473.0 | 3.3 |
| GRO | 1057.7 | 3534.0 | 3.3 |
| DR3/TNFRSF25 | 43.3 | 142.3 | 3.3 |
| EGF R/ErbB1 | 145.7 | 406.3 | 2.8 |
| IL-12 R beta 1 | 177.7 | 473.0 | 2.7 |
| IL-1 alpha | 1360.0 | 3331.0 | 2.4 |
| IL-17R | 832.0 | 1945.3 | 2.3 |
| IL-4 R | 8509.3 | 19494.3 | 2.3 |
| IL-8 | 1766.7 | 3823.3 | 2.2 |
| MCP-1 | 725.0 | 1548.3 | 2.1 |
| RANTES | 158.0 | 290.0 | 1.8 |
| Granzyme A | 1019.0 | 1717.0 | 1.7 |
| IL-5 | 1205.3 | 1996.3 | 1.7 |
| Kremen-2 | 391.0 | 622.0 | 1.6 |
| Osteoprotegerin/ | 4484.7 | 7127.3 | 1.6 |
| TNFRSF11B | | | |
| Siglec-9 | 43881.7 | 64277.7 | 1.5 |
| MIP-1b | 233.3 | 151.3 | −1.5 |
| Inhibin A | 210.0 | 134.0 | −1.6 |
| MCP-2 | 551.7 | 338.0 | −1.6 |
| TGF-beta 2 | 941.3 | 546.3 | −1.7 |
| TRAIL R1/DR4/ | 862.7 | 495.3 | −1.7 |
| TNFRSF10A | | | |
| NGF R | 217.3 | 123.3 | −1.8 |
| BMP-15 | 562.0 | 314.7 | −1.8 |
| BAFF R/TNFRSF13C | 413.7 | 228.7 | −1.8 |
| TRANCE | 270.3 | 147.7 | −1.8 |
| B7-1/CD80 | 961.3 | 508.7 | −1.9 |
| Neuropilin-2 | 565.0 | 294.7 | −1.9 |
| NT-4 | 415.0 | 209.0 | −2.0 |
| FGF Basic | 896.7 | 450.7 | −2.0 |
| MCP-3 | 587.7 | 291.7 | −2.0 |
| CTLA-4/CD152 | 557.3 | 271.3 | −2.1 |
| BD-1 | 250.0 | 117.3 | −2.1 |
| EGF | 1850.7 | 867.7 | −2.1 |
| IFN-alpha/beta R1 | 352.7 | 163.3 | −2.2 |
| VE-Cadherin | 412.0 | 187.7 | −2.2 |
| IL-2 R alpha | 1129.3 | 508.3 | −2.2 |
| Endoglin/CD105 | 1140.3 | 510.0 | −2.2 |
| PARC/CCL18 | 488.7 | 217.7 | −2.2 |
| CCR1 | 556.3 | 243.7 | −2.3 |
| Lymphotactin/XCL1 | 301.0 | 130.3 | −2.3 |
| TLR3 | 1029.3 | 445.3 | −2.3 |
| Lymphotoxin beta R/ | 271.0 | 116.3 | −2.3 |
| TNFRSF3 | | | |
| TIMP-4 | 477.7 | 201.0 | −2.4 |
| Adiponectin/Acrp30 | 4485.0 | 1860.3 | −2.4 |
| CCR2 | 510.3 | 209.3 | −2.4 |
| FADD | 282.0 | 115.7 | −2.4 |
| Vasorin | 372.0 | 152.0 | −2.4 |
| TRAIL/TNFSF10 | 513.7 | 208.7 | −2.5 |
| CXCR5/BLR-1 | 600.7 | 239.3 | −2.5 |
| IL-1 R4/ST2 | 1342.0 | 532.3 | −2.5 |
| LIF | 267.7 | 103.3 | −2.6 |
| VEGF-C | 430.7 | 165.0 | −2.6 |
| CCR4 | 639.0 | 244.7 | −2.6 |
| IL-2 R gamma | 396.3 | 151.3 | −2.6 |
| MMP-3 | 207.3 | 78.7 | −2.6 |
| Neurturin | 1021.7 | 381.3 | −2.7 |
| BMP-3 | 1039.0 | 387.3 | −2.7 |
| ICAM-1 | 100.7 | 36.3 | −2.8 |
| HVEM/TNFRSF14 | 123.3 | 43.7 | −2.8 |
| IL-22 R | 243.0 | 84.7 | −2.9 |
| WIF-1 | 882.7 | 301.3 | −2.9 |
| PDGF-BB | 203.7 | 67.7 | −3.0 |
| IFN-alpha/beta R2 | 509.3 | 164.7 | −3.1 |
| E-Selectin | 341.7 | 109.0 | −3.1 |
| Tie-1 | 231.7 | 73.3 | −3.2 |
| IGF-I SR | 932.0 | 287.3 | −3.2 |
| IL-1 R6/IL-1 Rrp2 | 501.3 | 154.0 | −3.3 |
| IL-3 R alpha | 610.7 | 174.7 | −3.5 |
| CCL28/VIC | 682.0 | 193.7 | −3.5 |
| IL-15 R alpha | 282.0 | 80.0 | −3.5 |
| NT-3 | 648.7 | 178.3 | −3.6 |
| Tie-2 | 5343.7 | 1468.0 | −3.6 |
| Angiopoietin-1 | 814.7 | 219.7 | −3.7 |
| MIP-3 alpha | 766.3 | 202.7 | −3.8 |
| GFR alpha-3 | 307.3 | 75.3 | −4.1 |
| Glut1 | 165.0 | 40.3 | −4.1 |
| PDGF-AB | 526.0 | 124.7 | −4.2 |
| CXCR3 | 1713.3 | 384.3 | −4.5 |
| DANCE | 395.7 | 86.7 | −4.6 |
| MFRP | 736.3 | 146.7 | −5.0 |
| CCR3 | 1279.0 | 240.0 | −5.3 |
| VEGF-B | 996.0 | 166.0 | −6.0 |
| CXCR4 (fusin) | 1138.3 | 183.3 | −6.2 |
| PLUNC | 137.0 | 20.3 | −6.7 |
| BLC/BCA-1/CXCL13 | 5564.3 | 422.7 | −13.2 |
| sFRP-4 | 173.3 | 12.7 | −13.7 |
| EMAP-II | 6165.7 | 383.0 | −16.1 |
| RANK/TNFRSF11A | 381.7 | 20.3 | −18.8 |
| CXCR2/IL-8 RB | 27292.0 | 1048.3 | −26.0 |
| IL-22 BP | 37.7 | 1.3 | −28.3 |
| VEGF-D | 13874.7 | 320.0 | −43.4 |

### Example 16

### Marker Identification for Other Cancers

In addition to stomach cancer, the computational techniques outlined above and additional tools have been applied to other cancers using publicly available cancer microarray data. For this study, microarray gene expression data for eight cancer types have been collected from databases on the Internet, liver cancer (Chen et al., 2002), prostate cancer (Lapointe et al., 2004), lung cancer (Garber et al., 2001), kidney cancer (Sarwal et al., 2001), colorectal cancer (Giacomini et al., 2005), breast cancer (Dairkee et al., 2004), ovarian cancer (Schaner et al., 2003) and pancreatic cancer (Iacobuzio-Donahue et al., 2003), each of which has a relatively large sample size.

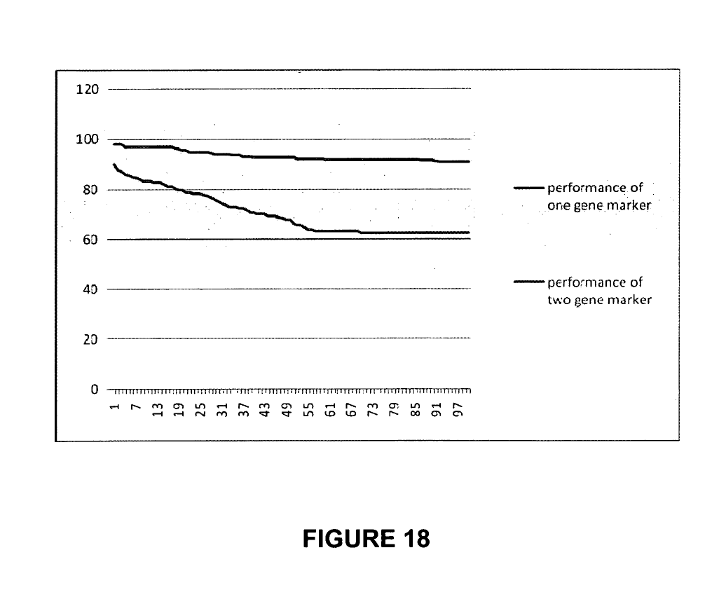

For each dataset, the top 100 markers that can best distinguish between cancer and reference tissues are predicted using one-, two-, three-, four- and five-genes as markers, using the same procedure outlined above. FIG. 18 shows the classification accuracy by the best one-gene and two-gene markers, respectively, in distinguishing between 83 prostate cancer tissues and 50 reference prostate tissues (two thirds of the data are used for training and the remaining one third for testing, using 5-cross validation). For prostate cancer, the best three one-gene markers are AMACR, ITPR1 and ACPP, with classification accuracies at 88.0%, 86.1% and 85.7%, respectively, and the best three two-gene markers are ITGA9-SPG3A, CREB3L4-ITGA9 and BLNK-ITGA9, with classification accuracies at 98.0% for all. An interesting observation is that the widely used PSA is ranked at the 167th position in our one-gene marker list in terms of its discerning power between cancer and the reference tissues. This is consistent with the accepted limitations of PSA in distinguishing between prostate cancer and benign prostatic hypertrophy. Among the top marker candidates, AMACR has recently been identified as a potential serum marker for prostate cancer by several groups (Bradford et al., 2006). Similar analyses were also done on seven other cancer types in the above list.

### Example 17

### Specificity Analysis of Predicted Gene Markers through Search against Public Microarray Data

To check if the predicted gene markers are specific to gastric cancer, a biomarker evaluation system has been developed, searching each predicted marker against public microarray datasets in the GEO (Barrett et al., 2005), Oncomine (Rhodes et al., 2004), and SMD (Sherlock et al., 2001) databases for human diseases. For each predicted marker, individual genes or groups of genes, along with their expression fold-change information, the following search was conducted. If a gene marker gives a substantial positive prediction (currently set at 30%) across multiple diseases, the marker is not considered specific to gastric cancer and hence is removed from the candidate list.

### Example 18

### Algorithm for Detecting Differentially Expressed Genes/transcripts

The goal of this study is to test the hypothesis (H₀) that a particular gene does not show k-fold change or more in expression level, across the majority of the patients (p-value<0.05). To check the hypothesis H₀ that a particular gene does not show certain expression level change in cancer, and the rejection of this hypothesis would mean an alternative holds for cancer. Let N[i] and C[i], i=1 . . . m, be the genes expressions in the reference and cancer tissues of i-th patient, and m be the number of all patients. If the hypothesis H₀ is true, then the probability P(N[i]>C[i])=P(N[i]<C[i])=0.5, assuming that gene's expression is a continuous random variable. Let K be a number of patients with N[i]/C[i]>0.5, then based on the Central Limit Theorem, the random variable K/m is approximately normal with mean=0.5 and a standard variation=0.5/\sqrt{m}, or X=2K/\sqrt{m} has a standard normal distribution N(0,1). Thus the p-value can be estimated as P(X>2Kₑₓₚ/\sqrt{m}), where Kₑₓₚ is the experimentally observed number of patients with P(N[i]<C[i]).

### Example 19

### Public Microarray Data of Gastric Cancer

To avoid the discrepancies caused by the bias of the sample distribution, two public microarray datasets for gastric cancer from the GEO database were downloaded for comparative studies: one (Kim dataset) (Kim et al., 2007) measures gene expression profiles of 50 gastric cancer patients in Korea, of diverse stage, cancer types, and the degree of cancer differentiation. The raw data is given by calculated log 2 fold change values for each tumor relative to the mean value of the normal sample; and the other one (Xin dataset, GSE2701) (Chen et al., 2003) measures gene expression of gastric patients tumor and normal tissues collected in Hong Kong, 126 in total, assayed using 44K human arrays against common reference (CRG). The first set has been normalization and log transformed, and we preprocessed Xin dataset by following the same procedure described in (Sharma et al., 2008).

The Kim dataset, with gene expression data of 50 gastric cancer patients in Korea, was used to evaluate the early stage markers, and the Xin dataset, with gene expression data of 100 gastric cancer and 24 reference tissues, was used to assess the generality of our proposed gene markers.

### Example 20

### Mapping Known Cis Regulatory Motifs for Splicing to Introns Immediately Before Skipped Exons

362 intronic cis regulatory motifs considered to be involved in splicing regulation have been collected (Wang et al., 2008). Studies in Wang et al., 2008, suggest that the immediate upstream intronic region (−150 to −30 nt relative to 5′ splicing site) of an exon enriched with such cis regulatory motifs generally indicates that the exon can be alternatively spliced. Further analysis suggests that a higher number of occurrences of such regulatory motifs are associated with higher occurrences of exon-skipping events of the exon. Hence, the occurrences of these regulatory motifs (100% sequence match) in the intronic region defined above for each exon have been counted.

All publications and patents mentioned in the above specification are herein incorporated by reference. Other embodiments of the invention will be apparent to those with knowledge in the art from consideration of the specification and practice of the invention disclosed herein. It is intended that the specification and examples be considered as exemplary only, with a true scope and spirit of the invention being indicated by the following claims.

### REFERENCES

Adkins J N, Varnum S M, Auberry K J, Moore R J, Angell N H, Smith R D, et al. Toward a human blood serum proteome: analysis by multidimensional separation coupled with mass spectrometry. Mol Cell Proteomics. 2002; 1(12):947-55.
Schrader M, Schulz-Knappe P. Peptidomics technologies for human body fluids. Trends Biotechnol. 2001; 19(10 Suppl):S55-60.
Tolson J, Bogumil R, Brunst E, Beck H, Elsner R, Humeny A, et al. Serum protein profiling by SELDI mass spectrometry: detection of multiple variants of serum amyloid alpha in renal cancer patients. Lab Invest. 2004; 84(7):845-56.
Holmila R, Fouquet C, Cadranel J, Zalcman G, Soussi T. Splice mutations in the p53 gene: case report and review of the literature. Hum Mutat. 2003; 21(1):101-2.
Li H R, Wang-Rodriguez J, Nair T M, Yeakley J M, Kwon Y S, Bibikova M, et al. Two-dimensional transcriptome profiling: identification of messenger RNA isoform signatures in prostate cancer from archived paraffin-embedded cancer specimens. Cancer Res. 2006; 66(8):4079-88.
Smith M W, Yue Z N, Geiss G K, Sadovnikova N Y, Carter V S, Boix L, et al. Identification of novel tumor markers in hepatitis C virus-associated hepatocellular carcinoma. Cancer Res. 2003; 63 (4): 859-64.
Young A N, de Oliveira Salles P G, Lim S D, Cohen C, Petros J A, Marshall F F, et al. Beta defensin-1, parvalbumin, and vimentin: a panel of diagnostic immunohistochemical markers for renal tumors derived from gene expression profiling studies using cDNA microarrays. Am J Surg Pathol. 2003; 27(2):199-205.
van de Vijver M J, He Y D, van't Veer L J, Dai H, Hart A A, Voskuil D W, et al. A gene-expression signature as a predictor of survival in breast cancer. N Engl J. Med. 2002; 347(25):1999-2009.
Resnick M B, Routhier J, Konkin T, Sabo E, Pricolo V E. Epidermal growth factor receptor, c-MET, beta-catenin, and p53 expression as prognostic indicators in stage 11 colon cancer: a tissue microarray study. Clin Cancer Res. 2004; 10(9):3069-75.
Sallinen S L, Sallinen P K, Haapasalo H K, Helin H J, Helen P T, Schraml P, et al. Identification of differentially expressed genes in human gliomas by DNA microarray and tissue chip techniques. Cancer Res. 2000; 60(23):6617-22.
Hendrix M J, Seftor E A, Meltzer P S, Gardner L M, Hess A R, Kirschmann D A, et al. Expression and functional significance of VE-cadherin in aggressive human melanoma cells: role in vasculogenic mimicry. Proc Natl Acad Sci USA. 2001; 98(14):8018-23. PMCID: 35460.
Menne K M, Hermjakob H, Apweiler R. A comparison of signal sequence prediction methods using a test set of signal peptides. Bioinformatics. 2000; 16(8):741-2.
Nair R, Rost B. Mimicking cellular sorting improves prediction of subcellular localization. J Mol. Biol. 2005; 348(1):85-100.
Horton P, Park K J, Obayashi T, Fujita N, Harada H, Adams-Collier C J, et al. WoLF PSORT: protein localization predictor. Nucleic Acids Res. 2007; 35(Web Server issue):W585-7.
Guda C. pTARGET: a web server for predicting protein subcellular localization. Nucleic Acids Res. 2006; 34(Web Server issue):W210-3.
Mott R, Schultz J, Bork P, Ponting C P. Predicting protein cellular localization using a domain projection method. Genome Res. 2002; 12(8):1168-74.
Smialowski P, Martin-Galiano A J, Mikolajka A, Girschick T, Holak T A, Frishman D. Protein solubility: sequence based prediction and experimental verification. Bioinformatics, 2007; 23(19):2536-42.
Chen Y, Zhang Y, Yin Y, Gao G, Li S, Jiang Y, et al. SPD—a web-based secreted protein database. Nucleic Acids Res. 2005; 33(Database issue):D169-73.
Tang Z Q, Han L Y, Lin H H, Cui J, Jia J, Low B C, et al. Derivation of stable microarray cancer-differentiating signatures using consensus scoring of multiple random sampling and gene-ranking consistency evaluation. Cancer Res. 2007; 67(20):9996-10003.
Lee Y, Kim B, Shin Y, Nam S, Kim P, Kim N, et al. ECgene: an alternative splicing database update. Nucleic Acids Res. 2007; 35(Database issue):D99-103. PMCID: 1716719.
Dantzig G B, A. Orden, and P. Wolfe. Generalized Simplex Method for Minimizing a Linear from Under Linear Inequality Constraints. Pacific Journal Math. 1999;Vol. 5:183-95.
Takeno, A., et al. Integrative approach for differentially overexpressed genes in gastric cancer by combining large-scale gene expression profiling and network analysis. Br J Cancer 99, 1307-1315 (2008).
El-Rifai, W., Frierson, H. F., Jr., Harper, J. C., Powell, S. M. & Knuutila, S. Expression profiling of gastric adenocarcinoma using cDNA array. Int J Cancer 92, 832-838 (2001).
Becker, K. F., et al. E-cadherin gene mutations provide clues to diffuse type gastric carcinomas. Cancer Res 54, 3845-3852 (1994).
Hippo, Y., et al. Global gene expression analysis of gastric cancer by oligonucleotide microarrays. Cancer Res 62, 233-240 (2002).
Moss, S. F., et al. Decreased expression of gastrokine 1 and the trefoil factor interacting protein TFIZ1/GKN2 in gastric cancer: influence of tumor histology and relationship to prognosis. Clin Cancer Res 14, 4161-4167 (2008).
Chen, X., et al. Variation in gene expression patterns in human gastric cancers. Mol Biol Cell 14, 3208-3215 (2003).
Dar, A. A., Belkhiri, A. & El-Rifai, W. The aurora kinase A regulates GSK-3beta in gastric cancer cells. Oncogene 28, 866-875 (2009).
Kim, K. R., et al. [Gene expression profiling using oligonucleotide microarray in atrophic gastritis and intestinal metaplasia]. Korean J Gastroenterol 49, 209-224 (2007).
Katayama, H., et al. Phosphorylation by aurora kinase A induces Mdm2-mediated destabilization and inhibition of p53. Nat Genet. 36, 55-62 (2004).
Chen, L., et al., Clinicopathological significance of overexpression of TSPAN1, Ki67 and CD34 in gastric carcinoma. Tumori, 2008. 94(4): p. 531-8.
Long, Y. M., et al., Nuclear factor kappa B: a marker of chemotherapy for human stage 1V gastric carcinoma. World J Gastroenterol, 2008. 14(30): p. 4739-44.
Yamada, Y., et al., Identification of prognostic biomarkers in gastric cancer using endoscopic biopsy samples. Cancer Sci, 2008. 99(11): p. 2193-9.
Silva, E. M., et al., Cadherin-catenin adhesion system and mucin expression: a comparison between young and older patients with gastric carcinoma. Gastric Cancer, 2008. 11(3): p. 149-59.
Xu, Y., L. Zhang, and G. Hu, Potential application of alternatively glycosylated serum MUC1 and MUC5AC in gastric cancer diagnosis. Biologicals, 2009. 37(1): p. 18-25.
Takeno, A., et al., Integrative approach for differentially overexpressed genes in gastric cancer by combining large-scale gene expression profiling and network analysis. Br J Cancer, 2008. 99(8): p. 1307-15.
Kon, O. L., et al., The distinctive gastric fluid proteome in gastric cancer reveals a multi-biomarker diagnostic profile. BMC Med Genomics, 2008. 1: p. 54.
Bernal, C., et al., Reprimo as a potential biomarker for early detection in gastric cancer. Clin Cancer Res, 2008. 14(19): p. 6264-9.
Taddei, A., et al., NF2 expression levels of gastrointestinal stromal tumors: a quantitative real-time PCR study. Tumori, 2008. 94(4): p. 551-5.
Ebert, M. P., et al., Overexpression of cathepsin B in gastric cancer identified by proteome analysis. Proteomics, 2005. 5(6): p. 1693-704.
Stefatic, D., et al., Optimization of diagnostic ELISA-based tests for the detection of auto-antibodies against tumor antigens in human serum. Bosn J Basic Med Sci, 2008. 8(3): p. 245-50.
Jin, B., et al., Detection of serum gastric cancer-associated MG7-Ag from gastric cancer patients using a sensitive and convenient ELISA method. Cancer Invest, 2009. 27(2): p. 227-33.
Ren, H., et al., Analysis of variabilities of serum proteomic spectra in patients with gastric cancer before and after operation. World J Gastroenterol, 2006. 12(17): p. 2789-92.
Peduzzi P, C. J., Feinstein A R, Holford T R Importance of events per independent variable in proportional hazards regression analysis. II. Accuracy and precision of regression estimates. Journal of Clinical Epidemiology 48, 1503-1510 (1995).
Chandanos, E. & Lagergren, J. Oestrogen and the enigmatic male predominance of gastric cancer. Eur J Cancer 44, 2397-2403 (2008).
Guojun Li, Q. M., Haibao Tang, Ying Xu. QUBIC: A Qualitative Biclustering Algorithm for Analyses of Gene Expression Data. (2009).
Dennis, G., Jr., et al. DAVID: Database for Annotation, Visualization, and Integrated Discovery. Genome Biol 4, P3 (2003).
Wu, J., Mao, X., Cai, T., Luo, J. & Wei, L. KOBAS server: a web-based platform for automated annotation and pathway identification. Nucleic Acids Res 34, W720-724 (2006).
Zhu, J., et al. The UCSC Cancer Genomics Browser. Nat. Methods 6, 239-240 (2009).
Schaefer, C. F., et al. PID: the Pathway Interaction Database. Nucleic Acids Res 37, D674-679 (2009).
Liu, R., et al. Mechanism of cancer cell adaptation to metabolic stress: proteomics identification of a novel thyroid hormone-mediated gastric carcinogenic signaling pathway. Mol Cell Proteomics 8, 70-85 (2009).
Bell, G. I., et al. Facilitative glucose transport proteins: structure and regulation of expression in adipose tissue. Int J Obes 15 Suppl 2, 127-132 (1991).
Wang, E. T., et al. Alternative isoform regulation in human tissue transcriptomes. Nature 456, 470-476 (2008).
Eyras, E., Caccamo, M., Curwen, V. & Clamp, M. ESTGenes: alternative splicing from ESTs in Ensembl. Genome Res 14, 976-987 (2004).
Kanehisa, M. a. G., S. KEGG: Kyoto Encyclopedia of Genes and Genomes. Nucleic Acids Res. 28, 27-30 (2000).
Cui, J., Liu, Q., Puett, D. & Xu, Y. Computational Prediction of Human Proteins That Can Be Secreted into the Bloodstream. Bioinformatics (2008).
Omenn G S, States D J, Adamski M, Blackwell T W, Menon R, Hermjakob H, et al. Overview of the HUPO Plasma Proteome Project: results from the pilot phase with 35 collaborating laboratories and multiple analytical groups, generating a core dataset of 3020 proteins and a publicly-available database. Proteomics. 2005; 5(13):3226-45.
Chen Y, Zhang Y, Yin Y, Gao G, Li S, Jiang Y, et al. SPD—a web-based secreted protein database. Nucleic Acids Res. 2005; 33(Database issue):D169-73.
Bateman A, Birney E, Cerruti L, Durbin R, Etwiller L, Eddy S, et al. The Pfam protein families database. Nucleic acids research. 2002; 30(1):276-80.
Reczko M, Bohr H. The DEF data base of sequence based protein fold class predictions. Nucleic Acids Res. 1994; 22(17):3616-9.
Bhasin M, Raghava G P. Classification of nuclear receptors based on amino acid composition and dipeptide composition. J Biol. Chem. 2004; 279(22):23262-6.
Platt J C. Fast Training of Support Vector Machines using Sequential Minimal Optimization. Advances in kernel methods: support vector learning. Cambridge, Mass., USA: MIT Press 1999. p. 185-208.
S. S. Keerthi SKS, C. Bhattacharyya, K. R. K. Murthy. Improvements to Platt's SMO Algorithm for SVM Classifier Design Neural Computation. 2001; 13:637-49.
Poola, I., et al. Identification of MMP-1 as a putative breast cancer predictive marker by global gene expression analysis. Nat Med 11, 481-483 (2005).
Ebert, M. P., et al. Overexpression of cathepsin B in gastric cancer identified by proteome analysis. Proteomics 5, 1693-1704 (2005).
Poon, T. C., et al. Diagnosis of gastric cancer by serum proteomic fingerprinting. Gastroenterology 130, 1858-1864 (2006).
Pieper R, Gatlin C, McGrath A, Makusky A, Mondal M, Seonarain M, Field E, Schatz C, Estock M, Ahmed N, al e (2004). Characterization of the human urinary proteome: a method for high-resolution display of urinary proteins on two-dimensional electrophoresis gels with a yield of nearly 1400 nearly protein spots. Proteomics, 1159-1174.
Castagna A, Cecconi D, Sennels L, Rappsilber J, Guerrier L, Fortis F, Boschetti E, Lomas L, Righetti P (2005). Exploring the hidden human urinary proteome via ligand library beads. J Proteome Res, 1917-1930.
Wang L, Li F, Sun W, Wu S, Wang X, Zhang L, Zheng D, Wnag J, Gao Y (2006). Concanavalin A captured glycoproteins in healthy human urine. Mol Cell Proteomics, 560-562.
Chang C-C, Lin C-J (2001). LIBSVM: a library for support vector machines.
Li Z R, Lin H H, Han L Y, Jiang L, Chen X, Chen Y Z (2006). PROFEAT: a web server for computing structural and physicochemical features of proteins and peptides from amino acid sequence. Nucleic Acids Res. 34, W32-37.
Prilusky J, Felder C E, Zeev-Ben-Mordehai T, Rydberg E H, Man O, Beckmann J S, Silman I, Sussman J L (2005). FoldIndex: a simple tool to predict whether a given protein sequence is intrinsically unfolded. Bioinformatics. 21, 3435-3438.
Gasteiger E, Gattiker A, Hoogland C, Ivanyi I, Appel R D, Bairoch A (2003). ExPASy: The proteomics server for in-depth protein knowledge and analysis. Nucleic Acids Res. 31, 3784-3788.
Bendtsen J D, Nielsen F I, Widdick D, Palmer T, Brunak S (2005). Prediction of twin-arginine signal peptides. BMC Bioinformatics. 6, 167.
Kall L, Krogh A, Sonnhammer E L (2007). Advantages of combined transmembrane topology and signal peptide prediction—the Phobius web server. Nucleic Acids Res. 35, W429-432.
Julenius K, Molgaard A, Gupta R, Brunak S (2005). Prediction, conservation analysis, and structural characterization of mammalian mucin-type O-glycosylation sites. Glycobiology. 15, 153-164.
Gupta R, Jung E, Brunak S (2004). Prediction of N-glycosylation sites in human proteins eds).
Eisenhaber F, Imperiale F, Argos P, Froemmel C (1995). Prediction of Secondary Structural Content of Proteins from Their Amino Acid Comosition Alone Utilizing Analytic Vector Decompositioned eds).
Mao X, Cai T, Olyarchuk J G, Wei L (2005). Automated Genome Annotation and Pathway Identification Using the KEGG Orthology (KO) As a Controlled Vocabulary. Bioinformatics, 3787-3793.
Ashkenas J, Muschler J, Bissell M (1996). The extracellular matrix in epithelial biology: Shared molecules and common themes in distant phyla. Dev Biol. 180, 433-444.
McKinnell R G, Parchment R E, Perantoni A, Damjanov I, Pierce G B (2006). The Biological Basis of Cancer. 2.
Stein G S, Pardee A B (2004). Cell cycle and Growth Control: Biomolecular Regulation and Cancer. 2.
Frixen U, Behrens J, Sachs M, Elberle G, Voss B, Warda A, Lochner D, Birchmeier W (1991). E-Cadherin-mediated cell-cell adhesion prevents invasiveness of human carcinoma cells. J Cell Biology. 113, 173-185.
de Visser K E, Eichten A, Coussens L M (2006). Paradoxical roles of the immune system during cancer development. Nat Rev Cancer. 6, 24-37.
Malumbres M, Barbacid M (2007). Cell cycle kinases in cancer. Curr Opin Genet Dev. 17, 60-65.
Greenman C, Stephens P, Smith R (2009). Patterns of Somatic Mutation in Human Cancer Genomes. Nature. 446, 153-158.
Sawyers C (2004). Targeted cancer therapy. Nature. 432, 294-297.
Cui J, Chen Y, Chou J, Sun L (2009). Biomarker Identification for Gastric Cancered eds): The University of Georgia.
Shimamura T, Ito H, Shibahara J, Watanabe A, Hippo Y, Taniguchi H, Chen Y, Kashima T, Ohtomo T, Tanioka F, Iwanari H, Kodama T, Kazui T, Sugimura H, Fukayama M, Aburatani H (2005). Overexpression of MUC13 is associated with intestinal-type gastric cancer. Cancer Sci. 96, 265-273.
Williams S J, Wreschner D H, Tran M, Eyre H J, Sutherland G R, McGuckin M A (2001). Muc13, a novel human cell surface mucin expressed by epithelial and hemopoietic cells. J Biol. Chem. 276, 18327-18336.
N'Dow J, Pearson J, Neal D (2004). Mucus production after transposition of intestinal segments into the urinary tract. World J. Urol. 22, 178-185.
Gelse K, Poschl E, Aigner T (2003). Collagens—structure, function, and biosynthesis. Adv Drug Deliv Rev. 55, 1531-1546.
Schmid T M, Linsenmayer T F (1987). Type X collagen. Orlando: Academic Press.
Ferguson D A, Muenster M R, Zang Q, Spencer J A, Schageman J J, Lian Y, Garner H R, Gaynor R B, Huff J W, Pertsemlidis A, Ashfaq R, Schorge J, Becerra C, Williams N S, Graff J M (2005). Selective identification of secreted and transmembrane breast cancer markers using Escherichia coli ampicillin secretion trap. Cancer Res. 65, 8209-8217.
Choi S Y, Hirata K, Ishida T, Quertermous T, Cooper A D (2002). Endothelial lipase: a new lipase on the block. J Lipid Res. 43, 1763-1769.
Ishida T, Choi S, Kundu R K, Hirata K, Rubin E M, Cooper A D, Quertermous T (2003). Endothelial lipase is a major determinant of HDL level. J Clin Invest. 111, 347-355.
Jin W, Millar J S, Broedl U, Glick J M, Rader D J (2003). Inhibition of endothelial lipase causes increased HDL cholesterol levels in vivo. J Clin Invest. 111, 357-362.
Ma K, Cilingiroglu M, Otvos J D, Ballantyne C M, Marian A J, Chan L (2003). Endothelial lipase is a major genetic determinant for high-density lipoprotein concentration, structure, and metabolism. Proc Natl Acad Sci USA. 100, 2748-2753.
Qiu G, Ho A C, Yu W, Hill J S (2007). Suppression of endothelial or lipoprotein lipase in THP-1 macrophages attenuates proinflammatory cytokine secretion. J Lipid Res. 48, 385-394.
Griffon N, Jin W, Petty T J, Millar J, Badellino K O, Saven J G, Marchadier D H, Kempner E S, Billheimer J, Glick J M, Rader D J (2009). Identification of the Active Form of Endothelial Lipase, a Homodimer in a Head-to-Tail Conformation. J Biol. Chem. 284, 23322-23330.
Chen X, Cheung S T, So S, Fan S T, Barry C, Higgins J, et al. Gene expression patterns in human liver cancers. Mol Biol Cell. 2002; 13(6):1929-39. PMCID: 117615.
Lapointe J, Li C, Higgins J P, van de Rijn M, Bair E, Montgomery K, et al. Gene expression profiling identifies clinically relevant subtypes of prostate cancer. Proc Natl Acad Sci USA. 2004; 101(3):811-6. PMCID: 321763.
Garber M E, Troyanskaya O G, Schluens K, Petersen S, Thaesler Z, Pacyna-Gengelbach M, et al. Diversity of gene expression in adenocarcinoma of the lung. Proc Natl Acad Sci USA. 2001; 98(24):13784-9. PMCID: 61119.
Sarwal M, Chang S, Barry C, Chen X, Alizadeh A, Salvatierra O, et al. Genomic analysis of renal allograft dysfunction using cDNA microarrays. Transplant Proc. 2001; 33(1-2):297-8.
Giacomini C P, Leung S Y, Chen X, Yuen S T, Kim Y H, Bair E, et al. A gene expression signature of genetic instability in colon cancer. Cancer Res. 2005; 65(20):9200-5.
Dairkee S H, Ji Y, Ben Y, Moore D H, Meng Z, Jeffrey S S. A molecular ‘signature’ of primary breast cancer cultures; patterns resembling tumor tissue. BMC Genomics. 2004; 5(1):47. PMCID: 509241.
Schaner M E, Ross D T, Ciaravino G, Sorlie T, Troyanskaya O, Diehn M, et al. Gene expression patterns in ovarian carcinomas. Mol Biol Cell. 2003; 14(11):4376-86. PMCID: 266758.
Iacobuzio-Donahue C A, Maitra A, Olsen M, Lowe A W, van Fleck N T, Rosty C, et al. Exploration of global gene expression patterns in pancreatic adenocarcinoma using cDNA microarrays. Am J. Pathol. 2003; 162(4):1151-62. PMCID: 1851213.
Bradford T J, Tomlins S A, Wang X, Chinnaiyan A M. Molecular markers of prostate cancer. Urol Oncol. 2006; 24(6):538-51.
Barrett T, Suzek T O, Troup D B, Wilhite S E, Ngau W C, Ledoux P, et al. NCBI GEO: mining millions of expression profiles—database and tools. Nucleic Acids Res. 2005; 33(Database issue):D562-6. PMCID: 539976.
Rhodes D R, Yu J, Shanker K, Deshpande N, Varambally R, Ghosh D, et al. ONCOMINE: a cancer microarray database and integrated data-mining platform. Neoplasia. 2004; 6(1):1-6. PMCID: 1635162.
Sherlock, G., et al. The Stanford Microarray Database. Nucleic Acids Res 29, 152-155 (2001).

1. A method for determining serum protein markers for the detection of cancer, the method comprising:
(a) obtaining a cancer sample and a reference sample;
(b) determining one or more genes that are differentially expressed between the cancer sample and the reference sample;
(c) identifying one or more proteins that are the products of said one or more genes;
(d) predicting the probability of the one or more proteins being secreted into a biological fluid; and
(e) detecting, in the biological fluid, the presence of the one or more proteins that are predicted to be secreted into the biological fluid,
wherein the detection of the one or more proteins in the biological fluid constitutes detection of cancer.

2. The method of claim 1, wherein the cancer sample or the reference sample comprise a tissue sample.

3. The method of claim 1, wherein there is an at least 1.5 fold change in the expression of the one or more genes between the cancer sample and the reference sample.

4. (canceled)

5. The method of claim 1, wherein the expression of the one or more genes is increased in the cancer sample as compared to the reference sample.

6. The method of claim 1, wherein the expression of the one or more genes is decreased in the cancer sample as compared to the reference sample.

7. The method of claim 1, wherein the determining of one or more genes that are differentially expressed between the cancer sample and the reference sample comprises isolating total RNA from the cancer sample and the reference sample.

8. (canceled)

9. The method of claim 1, further comprising identification of features of the one or more proteins that are differentially produced between the cancer sample and the reference sample.

10. The method of claim 9, wherein identification of the features of the one or more proteins that are differentially produced between the cancer sample and the reference sample comprises (a) identifying differentially expressed genes in the cancer sample versus the reference sample, (b) identifying differentially expressed splicing variants of genes in cancer versus reference sample, or (c) identifying marker genes that can distinguish between the cancer sample and the reference sample.

11. (canceled)

12. (canceled)

13. The method of claim 9, wherein the predicting comprises using the identified features of the one or more proteins that are differentially produced between the cancer sample and the reference sample, and wherein said features correspond to properties present in a set of proteins known to be secreted into the biological fluid.

14. (canceled)

15. (canceled)

16. (canceled)

17. (canceled)

18. (canceled)

19. The method of claim 1, wherein the detecting comprises mass spectrometric analysis of the biological fluid, western blot analysis of the biological fluid, or MS/MS analysis of the biological fluid.

20. (canceled)

21. (canceled)

22. (canceled)

23. (canceled)

24. (canceled)

25. (canceled)

26. (canceled)

27. The method of claim 1, wherein the biological fluid is one or more of serum, saliva, blood, urine, spinal fluid, seminal fluid, vaginal fluid, amniotic fluid, gingival crevicular fluid, or ocular fluid.

28. The method of claim 1, wherein the cancer includes gastric, pancreatic, lung, ovarian, liver, colon, colorectal, breast, nasopharynx, kidney, uterine cervical, brain, bladder, renal, and prostate cancers, melanoma, and squamous cell carcinoma.

29. The method of claim 1, wherein the proteins are human proteins.

30. A method of diagnosing a patient with cancer, comprising:
(a) obtaining a biological fluid from the patient; and
(b) detecting in the biological fluid, the presence of one or more marker proteins, wherein the one or more marker proteins are the products of one or more genes that are differentially expressed between a cancer sample and a reference sample, wherein the one or more marker proteins are predicted and experimentally validated to be secreted into biological fluid, and wherein the detection of the one or more marker proteins in the biological fluid constitutes detection of cancer.

31. (canceled)

32. The method of claim 31, wherein the differential expression comprises an increase in the levels of the one or more proteins in the biological fluid relative to the standard level.

33. The method of claim 31, wherein the differential expression comprises a decrease in the levels of the one or more proteins in the biological fluid relative to the standard level.

34. (canceled)

35. Markers for cancer identification comprising one or more proteins selected from the group consisting of MUC13, GKN2, COL10A, AZTP1, CTSB, LIPF, EL, and TOP2A, wherein the differential expression of the one or more proteins in a biological fluid obtained from a subject relative to a standard level is indicative of the occurrence of cancer in the subject.

36. The markers of claim 32, wherein the differential expression comprises an increase in the levels of the one or more proteins in the biological fluid relative to the standard level.

37. The markers of claim 32, wherein the differential expression comprises a decrease in the levels of the one or more proteins in the biological fluid relative to the standard level.

38. A kit for detecting cancer in a subject comprising:
(a) one or more first antibodies that specifically bind to proteins in the biological fluid, wherein the proteins are selected from the group consisting of MUC13, GKN2, COL10A, AZTP1, CTSB, LIPF, GIF, EL, and TOP2A;
(b) a second antibody that specifically binds to the one or more or the first antibodies; and optionally,
(c) a reference sample.
